(19) United States
(12) Patent Application Publication
(10) Pub. No.: US 2023/0086069 A1
(43) Pub. Date: Mar. 23, 2023

(54) ANTI-CD19 ANTIBODIES AND METHODS OF USING AND MAKING THEREOF

(71) Applicants: SYSTIMMUNE, INC., Redmond, WA (US); BAILI-BIO (CHENGDU) PHARMACEUTICAL CO., LTD., Chengdu, Sichuan (CN)

(72) Inventors: Soumili Chatterjee, Bothell, WA (US); Dennis R. Goulet, Redmond, WA (US); Andrew Waight, Kenmore, WA (US); Nga Sze Amanda Mak, Mukilteo, WA (US); Jahan KHALILI, Kirkland, WA (US); Yi ZHU, Chengdu, Sichuan (CN)

(21) Appl. No.: 17/909,357

(22) PCT Filed: Feb. 27, 2021
(86) PCT No.: PCT/US21/20145

§ 371 Date: Sep. 2, 2022

Related U.S. Application Data
(60) Provisional application No. 62/984,731, filed on Mar. 3, 2020.

(51) Int. Cl. C07K 16/28; A61K 47/68

(52) U.S. Cl. CPC C07K 16/2803; A61K 47/6849; A61K 47/6817; A61K 47/6803; C07K 2317/622; C07K 2317/92; C07K 2317/31; C07K 2317/55

(57) ABSTRACT

An isolated monoclonal antibody (mAb) or antigen-binding fragment thereof having a binding specificity to human CD19, wherein the isolated mAb or antigen-binding fragment comprises an amino acid sequence having an identity with a sequence selected from SEQ ID NO. 1, 3, 5, 7, 9, 11, 13, 15, 17, 19, 21, 23, 91, or 93, wherein the identity is not less than at least 95%.

### CROSS REFERENCE TO RELATED APPLICATIONS

This application claims the benefit of the filing date of U.S. Provisional Application Ser. No. 62/984,731 filed Mar. 3, 2020 under 35 U.S.C. 119(e), the entire disclosures of which are incorporated by reference herein.

### TECHNICAL FIELD

The present disclosure generally relates to the technical field of biologic therapeutics, and more particularly relates to making and using multi-specific antibodies.

### BACKGROUND

Lymphoma represents 4.3% of all cancers diagnosed annually in the United States, with B cell malignancies comprising approximately 90% of all lymphoma diagnoses. CD19 is a B-lymphocyte-specific member of the immunoglobulin superfamily expressed by B lymphocytes at different stages of differentiation, from the onset of V(D)J rearrangement until B cell maturation into plasma cells at which time the surface expression of CD19 seems to be lost. While CD19 is widely used as a pan-B cell marker, CD19 is found to be highly expressed in many forms of leukemia and lymphoma with characters of B-cell origins. CD19 has been a focus of immunotherapy development for over 30 years. Pharmaceutical companies are actively pursuing anti-CD19 strategies as they have the promise of directly targeting those B-cell malignancies corresponding to early B-cell differentiation stages. Targeting CD19 has been approved to be an excellent strategy of immune therapies, especially when the antibody therapies targeting CD22, another pan-B cell marker expressed by B-cell malignancies, were not successful.

CD19 is an important cell surface marker on normal B-cells and cancers of B-cell origins. As such it is highly desirable to have an antibody targeting CD19 for use in anti-cancer therapeutics. Reports in the literature demonstrate that it is difficult to identify anti-CD19 antibodies which also cross-react to the CD19 found in cynomolgus monkeys, a property which greatly facilitates therapeutic pharmacological and toxicological studies. The historic antibody BU12 has been shown to possess high affinity to human CD19 and cross reactivity to cynomolgus CD19, however this antibody was discovered from mouse hybridoma and does not comprise a human framework sequence. Therefore, a humanized variant of BU12 is highly desirable for therapeutic use.

### SUMMARY

The application provides anti-CD19 peptides, proteins, protein complexes, antibodies and methods of making and using thereof.

In one aspect, the application provides peptides having a binding specificity to human CD19. In one embodiment, the peptide has an amino acid sequence having at least 70%, 80%, 85%, 90%, 95%, 97%, 98% or 99% of sequence identity to SEQ ID NO. 1, 3, 5, 7, 9, 11, 13, 15, 17, 19, 21, 23, 91, or 93.

In one embodiment, the peptide is a scFv peptide. In one embodiment, the scFv peptide may have a binding affinity to human CD19 with a KD not greater than 1 nM, 2 nM, 3 nM, 5 nM 10 nM, 15 nM, 20 nM, 30 nM, 40 nM, or 50 nM.

In one aspect, the application provides an antibody or antigen-binding fragment thereof having a binding specificity to human CD19. In one embodiment, the isolated antibody or antigen-binding fragment comprises an amino acid sequence having at least 70%, 80%, 85%, 90%, 95%, 97%, 98% or 99% of sequence identity to SEQ ID NO. 1, 3, 5, 7, 9, 11, 13, 15, 17, 19, 21, 23, 91, or 93. In one embodiment, the antibody comprises an isolated monoclonal antibody (mAb).

In one embodiment, the antibody is a bi-specific antibody. In one embodiment, the antibody is a multi-specific antibody. In one embodiment, the antibody is a tri-specific antibody, tetra-specific antibody, penta-specific antibody, or hexa-specific antibody.

In one embodiment, the antibody comprises a scFv, wherein the scFv comprises an amino acid sequence having at least 70%, 80%, 85%, 90%, 95%, 97%, 98% or 99% of sequence identity to SEQ ID NO. 1, 3, 5, 7, 9, 11, 13, 15, 17, 19, 21, 23, 91, or 93.

In one embodiment, the antibody comprises a Fab, wherein the Fab comprises an amino acid sequence having at least 70%, 80%, 85%, 90%, 95%, 97%, 98% or 99% of sequence identity to SEQ ID NO. 1, 3, 5, 7, 9, 11, 13, 15, 17, 19, 21, 23, 91, or 93.

In one embodiment, the application provides a multi-specific antibody-like protein. In one embodiment, the protein comprises the peptide having an amino acid sequence having at least 70%, 80%, 85%, 90%, 95%, 97%, 98% or 99% of sequence identity to SEQ ID NO. 1, 3, 5, 7, 9, 11, 13, 15, 17, 19, 21, 23, 91, or 93. In one embodiment, the multi-specific antibody-like protein has a N-terminal and a C-terminal, comprising in tandem from the N-terminal to the C-terminal, a first binding domain (D1) at the N-terminal, a second binding domain (D2) comprising a light chain moiety, a Fc region, a third binding domain (D3), and a fourth binding domain (D4) at the C-terminal. The light chain moiety comprises a fifth binding domain (D5) covalently attached to the C-terminal, a sixth binding domain (D6) covalently attached to the N-terminal, or both, and the D1, D2, D3, D4, D5 and D6 each has a binding specificity to a tumor antigen, an immune signaling antigen, or a combination thereof.

In one embodiment, the multi-specific antibody-like protein is penta-specific. In one embodiment, the antibody-like protein comprises binding domains including D1, D2, D3, D4 and D6.

In one embodiment, the multi-specific antibody-like protein is hexa-specific.

In one embodiment, D1 comprises the peptide comprises an amino acid sequence having at least 70%, 80%, 85%, 90%, 95%, 97%, 98% or 99% of sequence identity to SEQ ID NO. 1, 3, 5, 7, 9, 11, 13, 15, 17, 19, 21, 23, 91, or 93.

In one embodiment D1 comprises a peptide having an amino acid sequence with 95% sequence identity to SEQ ID NO. 7 or 19.

In one embodiment, D2 comprises the peptide an amino acid sequence having at least 70%, 80%, 85%, 90%, 95%, 97%, 98% or 99% of sequence identity to SEQ ID NO. 1, 3, 5, 7, 9, 11, 13, 15, 17, 19, 21, 23, 91, or 93.

In one embodiment, D2 comprises a peptide having an amino acid sequence with 95% sequence identity to SEQ ID NO. 91 or 93.

In one embodiment, D6 comprises the peptide having an amino acid sequence having at least 70%, 80%, 85%, 90%, 95%, 97%, 98% or 99% of sequence identity to SEQ ID NO. 1, 3, 5, 7, 9, 11, 13, 15, 17, 19, 21, 23, 91, or 93.

In one embodiment, D6 comprises a peptide having an amino acid sequence with 95% sequence identity to SEQ ID NO. 7 or 19.

In one embodiment, the application provides multi-specific monoclonal antibody, comprising the multi-specific antibody-like protein as claimed herein.

In one embodiment, the multi-specific monoclonal antibody may have a binding affinity to human CD19 with a Kd not greater than 1 nM, 5 nM, 10 nM, 20 nM, 30 nM, 40 nM or 50 nM.

In one embodiment, the antibody is a humanized antibody. In one embodiment, the multi-specific monoclonal antibody is an IgG.

In one embodiment, the application provides isolated nucleic acid encoding the isolated mAb or an antigen-binding fragment, the IgG1 heavy Chain, the kappa light chain, the variable light chain, or the variable heavy chain, as disclosed thereof.

In one aspect, the application provides isolated nucleic acid sequence encoding an amino acid sequence of the multi-specific monoclonal antibody as disclosed herein.

In one embodiment, the application provides an expression vector comprising the isolated nucleic acid, as disclosed thereof.

In one embodiment, the application provides host cells comprising the nucleic acid as disclosed thereof. In one embodiment, the host cell is a prokaryotic cell or a eukaryotic cell.

In one aspect, the application provides methods of producing an antibody comprising culturing the host cell so that the antibody is produced.

In one aspect, the application provides immuno-conjugates. In one embodiment, the immunoconjugate comprises the isolated mAb or an antigen-binding fragment thereof and a drug unit, wherein the drug unit is linked to the isolated mAb or an antigen-binding fragment through a linker, and wherein the linker comprises a covalent bond selected from an ester bond, an ether bond, an amine bond, an amide bond, a disulphide bond, an imide bond, a sulfone bond, a phosphate bond, a phosphorus ester bond, a peptide bond, a hydrazone bond or a combination thereof.

In one embodiment, the drug unit comprises a cytotoxic agent, an immune regulatory reagent, an imaging agent or a combination thereof. In one embodiment, the cytotoxic agent is selected from a growth inhibitory agent or a chemotherapeutic agent from a class of tubulin binders, DNA intercalators, DNA alkylators, enzyme inhibitors, immune modulators, antimetabolite agents, radioactive isotopes, or a combination thereof. In one embodiment, the cytotoxic agent is selected from a calicheamicin, camptothecin, ozogamicin, monomethyl auristatin E, emtansine, a derivative or a combination thereof.

In one embodiment, the immune regulatory reagents activate or suppress immune cells, T cell, NK cell, B cell, macrophage, or dendritic cell. In one embodiment, the imaging agent may be radionuclide, a florescent agent, a quantum dots, or a combination thereof.

In one aspect, the application provides pharmaceutical composition. In one embodiment, the pharmaceutical composition comprises the isolated mAb or an antigen-binding fragment thereof a pharmaceutically acceptable carrier. In one embodiment, the pharmaceutical composition may further include a chemotherapeutic agent, a growth inhibitory agent, a cytotoxic agent from class of calicheamicin, an antimitotic agent, a toxin, a radioactive isotope, a therapeutic agent, or a combination thereof.

In one aspect, the application provides a pharmaceutical composition including an immune-conjugate as disclosed herein and a pharmaceutically acceptable carrier.

In one aspect, the application provides methods of treating a subject with a cancer. In one embodiment, the method comprises administering to the subject an effective amount of the isolated mAb or an antigen-binding fragment as disclosed thereof. In one embodiment, the method may further include co-administering an effective amount of a therapeutic agent, wherein the therapeutic agent comprises an antibody, a chemotherapy agent, an enzyme, or a combination thereof. In one embodiment, the subject is a human.

In a further aspect, the application provides a solution comprising an effective concentration of the multi-specific monoclonal antibody as disclosed herein. In one embodiment, the solution is blood plasma in a subject.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments arranged in accordance with the disclosure and are, therefore, not to be considered limiting of its scope, the disclosure will be described with additional specificity and detail through use of the accompanying drawings, in which:

FIG. 1 shows an increase in humanness scores (Z-score) from mouse sequence (grey line) to the humanized framework (dark line) in the variable regions of humanized BU12, H4 (Vk for kappa light chain in 1A, and VH for heavy chain in 1B) and H5 (Vk for kappa light chain in 1C and VH for heavy chain in 1D);

FIG. 2 shows the sequence alignment of the variable regions (VL for light chain in 2A and VH for heavy chain in 2B) of humanized mouse BU12 (H1-H6, and H7) and human antibody (21D4);

FIG. 3 shows DLS thermal stability for SI-63C1 (BU12-chimeric), SI-63C2 (humanized BU12, H1) and SI-34C1 (human antibody, 21D4);

FIG. 4 shows the histograms depicting the cross reactivity of the SI-63C2 antibody to human, cynomolgus, and rhesus CD20+ B cells (4A) and CD20− lymphocytes (4B);

FIG. 5 shows the dose-response curve of the SI-63C2 antibody binding to human (5A), cynomolgus (5B), and rhesus (SC) CD20+ lymphocytes, as compared to its parental control (SI-63C1) and mouse anti-human CD19 antibody controls (SJ25C, LT19, HIB19, and 4G7);

FIG. 6 shows an analytical SEC profile of SI-63R1(H1), the protein-A purified recombinant anti-CD19 scFv-HIS protein (6A) and DLS Thermal Stability of SI-63R1(H1) with unfolding temperature at about 58.8° C. (6B);

FIG. 7 shows analytical SEC profile of the protein-A purified recombinant anti-CD19 scFv-monoFc proteins (H1 through H6) with 90% protein of interest (POI);

FIG. 8 depicts a schematic diagram of six binding domains (D1-D6) in hexaGNC antibodies that comprise the core Fab (D2) and Fc regions and the additional D1, D3, and D4 on heavy chain (HC) and D5 and D6 on light chain;

FIG. 9 shows the ExpiCHO expression and purification of three hexaGNC antibodies SI-77H3, SI-77H6, and SI-55H11 with their humanized anti-CD19 domains, H4 at D1, H7 at D2 (Fab), and H4 at D6, respectively;

FIG. 10 shows the dose-response curves of an antibody (SI-38E17, SI-55H11, SI-77H3, and SI-77H6, respectively) direct cellular cytotoxicity (ADCC) to either human (10A) or cynomolgus (10B) PBMC; and

FIG. 11 shows that the humanized CD19 binding domain of hexaGNC antibodies, such as SI-77H, SI-77H6, and SI-55H11, mediates the cytolysis of Raji lymphoma cells that express only CD19 but no other tumor antigens (11A) with a potent dose-response curve comparable to that of SI-38E17, a human anti-CD19 antibody (21D4) (11B).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure provides, among others, isolated antibodies, methods of making such antibodies, monoclonal and/or recombinant monospecific antibodies, multi-specific antibodies, antibody-drug conjugates and/or immuno-conjugates composed from such antibodies or antigen binding fragments, pharmaceutical compositions containing the antibodies, monoclonal and/or recombinant monospecific antibodies, multi-specific antibodies, antibody-drug conjugates and/or immuno-conjugates, the methods for making the antibodies and compositions, and the methods for treating cancer using the antibodies and compositions disclosed herein. Specifically, the present disclosure provides isolated monoclonal antibodies (mAb) or antigen-binding fragments thereof having a binding specificity to human CD19 (Table 1), wherein the isolated mAb or antigen-binding fragments comprise an amino acid sequence having an identity with a sequence selected from SEQ ID NO. 1, 3, 5, 7, 9, 11, 13, 15, 17, 19, 21, 23, 91, or 93.

The terms “a”, “an” and “the” as used herein are defined to mean “one or more” and include the plural unless the context is inappropriate.

The terms “polypeptide”, “peptide”, and “protein”, as used herein, are interchangeable and are defined to mean a biomolecule composed of amino acids linked by a peptide bond.

The term “antigen” refers to an entity or fragment thereof which can induce an immune response in an organism, particularly an animal, more particularly a mammal including a human. The term includes immunogens and regions thereof responsible for antigenicity or antigenic determinants.

The terms “antigen- or epitope-binding portion or fragment”, “variable region”, “variable region sequence”, or “binding domain” refer to fragments of an antibody that are capable of binding to an antigen (such as CD19 in this application). These fragments may be capable of the antigen-binding function and additional functions of the intact antibody. Examples of binding fragments include, but are not limited to, a single-chain Fv fragment (scFv) consisting of the variable light chain (VL) and variable heavy chain (VH) domains of a single arm of an antibody connected in a single polypeptide chain by a synthetic linker, or a Fab fragment which is a monovalent fragment consisting of the VL, constant light (CL), VH and constant heavy 1 (CH1) domains. Antibody fragments can be even smaller sub-fragments and can consist of domains as small as a single CDR domain, in particular the CDR3 regions from either the VL and/or VH domains (for example see Beiboer et al., J. Mol. Biol. 296:833-49 (2000)). Antibody fragments are produced using conventional methods known to those skilled in the art. The antibody fragments can be screened for utility using the same techniques employed with intact antibodies.

The “antigen- or epitope-binding portion or fragment”, “variable region”, “variable region sequence”, or “binding domain” may be derived from an antibody of the present disclosure by a number of art-known techniques. For example, purified monoclonal antibodies can be cleaved with an enzyme, such as pepsin, and subjected to HPLC gel filtration. Papain digestion of antibodies produces two identical antigen binding fragments, called “Fab” fragments, each with a single antigen binding site, and a residual “Fc” fragment, whose name reflects its ability to crystallize readily. Pepsin treatment yields an F(ab′)₂ fragment that has two antigen combining sites and is still capable of cross-linking antigen. The appropriate fraction containing Fab fragments can then be collected and concentrated by membrane filtration and the like. For further description of general techniques for the isolation of active fragments of antibodies, see for example, Khaw, B. A. et al. J. Nucl. Med. 23:1011-1019 (1982); Rousseaux et al. Methods Enzymology, 121:663-69, Academic Press, 1986.

The term “antibody” is used in the broadest sense and specifically covers single monoclonal antibodies and/or recombinant antibodies (including agonist and antagonist antibodies), antibody compositions with polyepitopic specificity, as well as antibody fragments (e.g., Fab, F(ab′)₂, and Fv), so long as they exhibit the desired biological activity. In some embodiments, the antibody may be monoclonal, polyclonal, chimeric, single chain, multi-specific or multi-effective, human and humanized antibodies, as well as active fragments thereof. Examples of active fragments of molecules that bind to known antigens include Fab, F(ab′)₂, scFv and Fv fragments, including the products of a Fab immunoglobulin expression library and epitope-binding fragments of any of the antibodies and fragments mentioned above.

The term “Fv” refers to the minimum antibody fragment which contains a complete antigen recognition and binding site. This region consists of a dimer of one heavy and one light chain variable domain in tight, non-covalent association. It is in this configuration that the three CDRs of each variable domain interact to define an antigen binding site on the surface of the VH-VL dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

In some embodiments, antibody may include immunoglobulin molecules and immunologically active portions of immunoglobulin molecules, i.e. molecules that contain a binding site and that immunospecifically bind an antigen. A typical antibody refers to heterotetrameric protein comprising typically of two heavy (H) chains and two light (L) chains. Each heavy chain is comprised of a heavy chain variable domain (abbreviated as VH) and a heavy chain constant domain. Each light chain is comprised of a light chain variable domain (abbreviated as VL) and a light chain constant domain. The light chains of antibodies (immunoglobulins) from any vertebrate species can be assigned to one of two clearly distinct types, called kappa and lambda, based on the amino acid sequences of their constant domains. The VH and VL regions can be further subdivided into domains of hypervariable complementarity determining regions (CDR), and more conserved regions called framework regions (FR). Each variable domain (either VH or VL) is typically composed of three CDRs and four FRs, arranged in the following order: FR1, CDR1, FR2, CDR2, FR3, CDR3, FR4 from amino-terminus to carboxy-terminus. Within the variable regions of the light and heavy chains there are binding regions that interacts with the antigen.

Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called alpha, delta, epsilon, gamma, and mu, respectively. The subunit structures and three-dimensional configurations of different classes of immunoglobulins are well known.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler & Milstein, Nature, 256:495 (1975), or may be made by recombinant DNA methods (see, e.g., U.S. Pat. No. 4,816,567). “Recombinant” means the antibodies are generated using recombinant nucleic acid techniques in exogeneous host cells.

Monoclonal antibodies can be produced using various methods, including without limitation, mouse hybridoma, phage display, recombinant DNA, molecular cloning of antibodies directly from primary B cells, and antibody discovery methods (see Siegel. Trans fus. Clin. Biol. 2002; Tiller. New Biotechnol. 2011; Seeber et al. PLOS One. 2014). Monoclonal antibodies may include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 [1984]).

The term “multi-specific” antibody as used herein denotes an antibody that has at least two binding sites each having a binding affinity to an epitope of an antigen. The term “bi-specific, tri-specific, tetra-specific, penta-specific, or hexa-specific” antibody as used herein denotes an antibody that has two, three, four, five, or six antigen-binding sites. For example, the antibodies disclosed herein with five binding sites are penta-specific, with six binding sites are hexa-specific.

The term “guidance and navigation control (GNC)” protein refers to a multi-specific protein capable of binding to at least one effector cell (such as immune cell) antigen and at least one target cell (such as tumor cell, immune cell, or microbial cell) antigen. The GNC protein may adopt an antibody-core structure including a Fab region and Fc region with various binding domains attached to the antibody-core, in which case the GNC protein is also termed GNC antibody. The GNC protein may adopt an antibody-like structure, in which case the Fv fragment may be replaced with a non-antibody based binding domain such as NKG2D, 4-1BBL (a 4-1BB receptor ligand), 4-1BBL trimer for 4-1BB, or a receptor.

The term “GNC antibody” refers to a GNC protein had an antibody structure that is capable of binding to at least one effector cell (such as immune cell) and at least one target cell (such as tumor cell, immune cell, or microbial cell) simultaneously. The term “biGNC, triGNC, tetraGNC, pentaGNC, or hexaGNC” antibody as used herein denotes a GNC antibody that has two, three, four, five, or six antigen-binding sites, of which at least one antigen-binding site has the binding affinity to an immune cell and at least one antigen-binding site has the binding affinity to a tumor cell. In one embodiment, the GNC antibodies disclosed herein have four to six binding sites (or binding domain) and are tetraGNC, pentaGNC, and hexaGNC antibodies, respectively. In some embodiments, the GNC antibodies include antibody binding domains (such as Fab and scFv) without the requirement for additional protein engineering in the Fc region. In one embodiment, the GNC antibodies additionally have the advantage of retaining bivalency for each targeted antigen. Further in one embodiment, the GNC antibodies have the advantage of avidity effects that result in higher affinity for antigens and slower dissociation rates. This bivalency for each antigen is in contrast to many multi-specific platforms that are monovalent for each targeted antigen, and thus often lose the beneficial avidity effects that make antibody binding so strong.

The term “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity. Methods to obtain “humanized antibodies” are well known to those skilled in the art. (see, e.g., Queen et al., Proc. Natl Acad Sci USA, 86:10029-10032 (1989), Hodgson et al., Bio/Technology, 9:421 (1991)).

The terms “isolated” or “purified” refers to a biological molecule free from at least some of the components with which it naturally occurs. Either “Isolated” or “purified,” when used to describe the various polypeptides disclosed herein, means a polypeptide that has been identified and separated and/or recovered from a cell or cell culture from which it was expressed. Ordinarily, a purified polypeptide will be prepared by at least one purification step. An “isolated” or a “purified” antibody refers to an antibody which is substantially free of other antibodies having different antigenic a binding specificity.

The term “immunogenic” refers to substances which elicit or enhance the production of antibodies, T-cells or other reactive immune cells directed against an immunogenic agent and contribute to an immune response in humans or animals. An immune response occurs when an individual produces sufficient antibodies, T-cells and other reactive immune cells against administered immunogenic compositions of the present disclosure to moderate or alleviate the disorder to be treated. While the immunogenic response generally includes both cellular (T cell) and humoral (antibody) arms of the immune response, antibodies directed against therapeutic proteins (anti-drug antibodies, ADA) may consist of IgM, IgG, IgE, and/or IgA isotypes.

The terms “specific binding”, “specifically binds to”, or “is specific for a particular antigen or an epitope” means that the binding is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10 M, alternatively at least about 10⁻¹⁰ M, at least about 10⁻¹¹ M, at least about 10⁻² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction.

The present disclosure may be understood more readily by reference to the following detailed description of specific embodiments and examples included herein. Although the present disclosure has been described with reference to specific details of certain embodiments thereof, it is not intended that such details should be regarded as limitations upon the scope of the disclosure.

### EXAMPLES

### Example 1. Designing Humanized Anti-CD19 Sequences

All computational steps were performed in the Discovery Studio package (Dassault Systems). First, a structural model was generated using the mouse BU12 sequence (McDonagh et al., 2009). Antibody framework regions in the input sequence were identified and aligned to a database of antibody variable domains using Hidden Markov Models (HMM), and this alignment was used to build and score models using the MODELLER software. CDR loop modelling was performed by a structural mapping of the CDRL1, CDRL2, CDRL3, CDRH1, and CDRH2 regions to known canonical classes and loop models were built similarly to the framework.

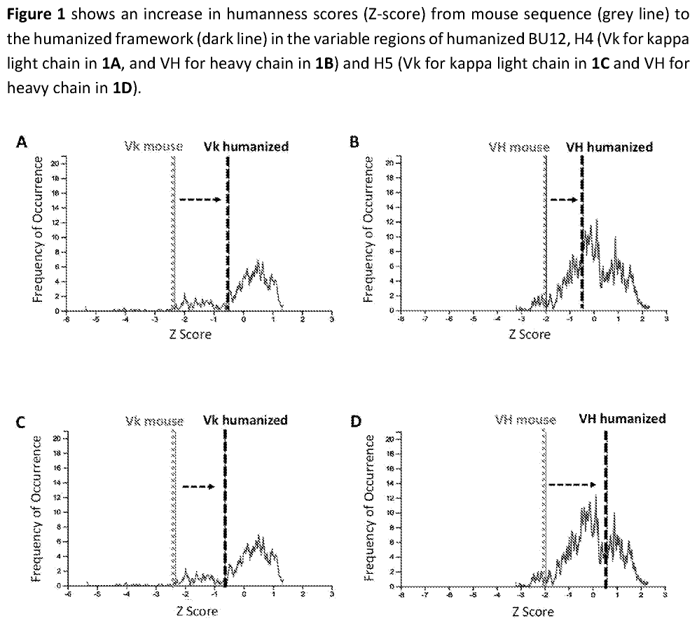

The framework regions from the mouse BU12 antibody were aligned and matched to the closest human germline sequence, and CDRs regions were copied into the human sequence except for important structural residues (Vernier residues [Almagro and Fransson, 2008]). Mutations predicted to stabilize the previously build structural model were evaluated computationally by 1000 steps of Steepest Descent with a RMS gradient tolerance of 3, followed by Conjugate Gradient minimization and stabilizing mutations matching frequent human residues were chosen based on individual and combined −ΔΔG versus the initial model. Mutational stabilization energy analysis on discovery studio was performed by using sequence H1 as the reference. Version H2, H3 and H4 are mutational variants which had negative values for mutational energy (ΔΔG was −0.8, −1.5, and −1.1 kCal, respectively) and hypothesized to be more stable than version H1. The resulting humanized sequence (H1, SEQ ID NO. 1 and 13) was tested for humanness using the Abysis webserver based on the method of Abhinandan and Martin (2007). Using H4 as an example for its light chain (Vk) and heavy chain (VH) as shown in FIGS. 1A and 1B, the humanized sequences show a higher humanness score than the corresponding mouse sequences (BU12) (SEQ ID NO. 25 and 27).

In addition, a direct CDR grafting approach was used to generate humanized versions H5. A reference antibody framework was mutated to analogous human germline residues and CDRs were directly grafted in the mutated framework to generate H5. Using the resulting humanized sequence H5 as the founding sequence, more mutation was made on the H5 framework to improve framework stability which generated humanized sequence (H6). The humanized sequence H5 (SEQ ID NO. 9 and 21) was tested for humanness using the Abysis webserver based on the method of Abhinandan and Martin (2007). The humanized sequences show a higher humanness score than the mouse sequence (BU12) (SEQ ID NO. 25, and 27) (FIGS. 1C and 1D).

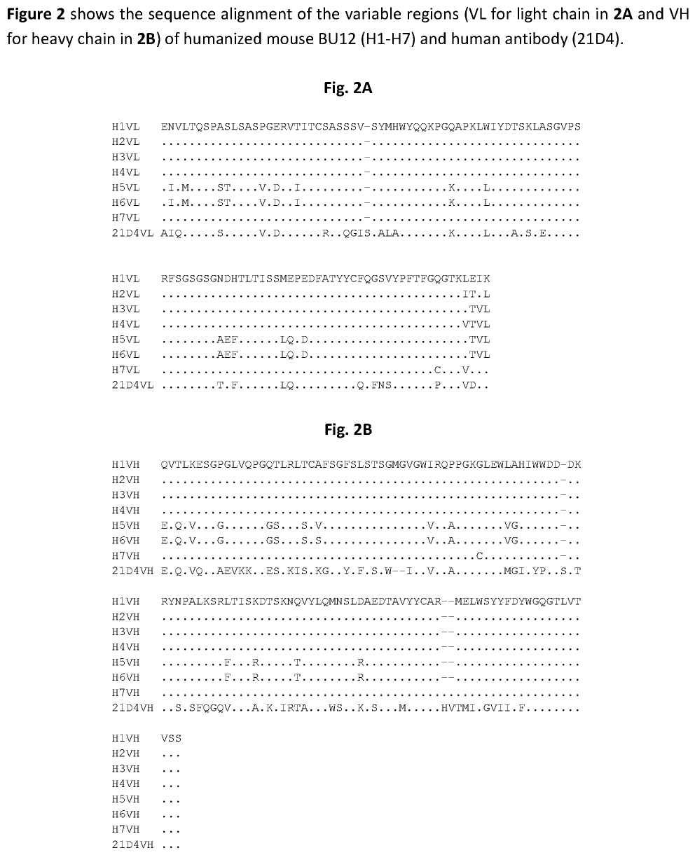

H1 is the first humanized version originating directly from the variable domains of the Fab sequence of BU12 with a signature amino acid sequence, LEIK, at the C-terminus. As shown in FIG. 2, the last three residues (EIK) are predominantly present for variable kappa chains present in nature and provides stability when positioned specifically in the Fab domain. In this context, having H4 that ends with VTVL at the Fab position may not be ideal for the stability of the antibody. Version H7 is a modified H4 which is hypothesized to improve protein stability when positioned in the context of a Fab domain (https://www.ncbi.nlm.nih.gov/pmc/articles/PMC5058631/). H7 was created by restoring EIK and introducing a disulfide staple between H7VL and H7VH via a Q to C and a G to C mutation, respectively.

To compare and prioritize these designed sequences, all humanized variable regions (H1, H2, H3, H4, H5, H6, and H7) were aligned with the sequences from a human anti-CD19 antibody, 21D4 (Rao-Naik et al., 2009), as shown in FIG. 2. The percentage identities to H1 are 98.1% VL and 99.1-100% VH for H2, H3, H4, and H7, 85% VL and 86% VH for H5 and H6, and 70% VL and 51% VH for 21D4. These findings imply that there are substantial flexibilities in the primary sequences of an anti-CD19 binding domain.

To predict immunogenicity of humanized anti-CD19 sequences, MixMHC2pred algorithm (Gfeller Lab, https://github.com/GfellerLab/MixMHC2pred) was used to predict the extent of major histocompatibility complex-11 (MHC-II) binding of peptides within mouse and humanized (VH/VL) sequences. The algorithm detects the number of ‘core’ peptides in a given amino acid sequence that will bind to MCHII with sufficient affinity to form a T cell epitope. The higher the number of MHCII-binding peptides identified in a sequence, the more potential T cell epitopes the sequence contains. A high number of core peptides increases the likelihood of containing some peptides that are pro-immunogenic. Reducing the number of core peptides in the antibody variable regions may thus help to reduce ADA by eliminating potential T cell epitopes.

Anti-CD19 variable sequences were run through the MixMHC2pred algorithm as scFv (VH-(G4S)4-VL). The algorithm includes the option to score among multiple alleles. In this case, “the score from each peptide is taken as its best percentile rank among all the alleles.” This scoring strategy allows sequences to examined to find the strongest ligands to any allele of MHCII. For sequence analysis of antibody variable domains, the number of core peptides was calculated based on the number of peptides in the sequence that could bind to any MHCII allele with a score in the top 0.2% of interactions. As shown in Table 1, most of humanized sequences have lower scores than their parental mouse sequences, indicative of weaker MHCII binding peptides and lower risk of immunogenicity. Herein, the total score of core peptides in the variable regions that were predicted to bind strongly to MHCII decreased from 9 for mouse sequences to 5 for humanized sequences (H1 through H4, and H7, no change for H5 and H6). The humanness scores for light and heavy chains were calculated using the humanness Z score analysis algorithm (Abhinandan & Andrew, 2007). For VH sequences, versions H1-H4 had similar humanness as 21D4, while H5 and H6 had higher humanness and H7 had lower humanness. For VK sequences, H1-H7 all had similar humanness which was slightly lower than that of 21D4. Notably, all humanized sequences (H1-H7, VH and Vk) had significantly higher humanness scores than the original mouse sequences (Table 1). Considering both humanness and MHC-II peptide binding scores, H1-H4 and H7 were the candidates for generating humanized anti-CD19 antibodies.

### Example 2. Expression of Humanized Anti-CD19 Monoclonal Antibodies

To characterize the humanized light chain CDR and heavy chain CDR and framework regions, the DNA sequences encoding H1 and other peptides were synthesized in overlapping fragments and cloned into linearized pTT5 vector (NE Builder) containing a C-terminal human kappa sequence, or human IgG CH1 and Fc region respectively to create a mAb format (SEQ ID NO. 37 and 39). The DNA sequences for 21D4 and Mouse (BU12) variable regions were also synthesized and cloned into linearized pTT5 vector containing a C-terminal human kappa sequence, or IgG CH1 and Fc region respectively to generate a chimeric mAb format (SEQ ID NO. 33, 35, 83, and 85). The plasmid DNA containing the antibody sequences were expressed using the ExpiCHO expression system (ThermoFisher). The three recombinant antibodies, SI-63C1 (with BU12 mouse parental anti-CD19 variable sequence), SI-63C2 (with H1 humanized anti-CD19 variable sequence, also known as SI-huCD19), and SI-34C1 (with 21D4 human anti-CD19 variable sequence), were purified from the culture supernatant by using a Protein-A affinity chromatography column (mabSelect Resin, Ge healthcare) with PBS (5×Cv) for washing followed by 20 mM Glycine pH 3.5 for elution. The resulting proteins were neutralized with 100× Tris pH 8.5 and dialyzed overnight into PDB buffer. To check for stability and monodispersity, purified antibodies were concentrated to 1 mg/ml and injected onto an analytical HPLC (waters, column waters BEH200A 300 mm column). The purified anti-CD19 antibodies showed a sharp monodispersed peak with the correct size with 1.8-2.5% aggregate (Table 2).

### Example 3. Characterization of SI-63C2

The purified SI-63C1, SI-63C2, and SI-34C1 antibodies were tested for their binding affinity using biolayer interferometry (ForteBio OctetRED 384). The antibodies were bound to anti-human Fc biosensors, and human CD19 protein (R&D Biosystems Cat #9269-CD-050) was used as the analyte in a 4-point series of 2-fold dilutions with the highest concentration starting at 200 nM. The results of Octet analysis indicated that the binding affinity of SI-63C3 (also known as SI-huCD19) to human and cynomolgus CD19 were at 3.8 nM and 3.6 nM, comparable to that of the human anti-CD19 antibody (21D4) at 2.1 nM and 3.8 nM, respectively. Furthermore, the humanized anti-CD19 variable sequences of SI-63C2 not only retain the binding specificity to human and cynomolgus CD19 but also exhibited comparable binding affinity (KD) to SI-63C1 (with BU12 variable sequences) and SI-34C1 (with 21D4 variable sequences) (Table 2).

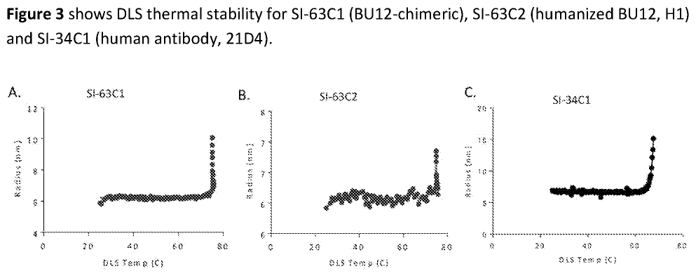

To test the thermal stability of SI-63C2, dynamic light scattering was used while the temperature was ramped from 25° C. to 75° C. at 0.5° C./min, and the radius of the proteins (1 mg/ml) was monitored by using Wyatt DynaPro Plate Reader III. As shown in FIG. 3 and Table 2, the results indicated that SI-63C2 and SI-63C1 displayed similar unfolding temperature, as measured by DLS™, which was higher than that of SI-34C1.

### Example 4. The Binding Specificity of SI-63C2

Non-human primates (NHPs), such as the cynomolgus or rhesus macaque, are currently necessary to provide risk assessment data for antibody drug development because of their similarity to humans, predictable metabolic stability, and historically established toxicity profiles. To minimize the use of NHPs and to increase the efficiency, antibody drug candidate should have high target specificity and cross-reactivity. In this context, CD19 is a pan-B cell marker and is expressed by the majority of malignant B cells. CD19 has a broader coverage to B cell development and differentiation than CD20, which is another pan-B cell marker for lymphocytes from human and NHPs, such as cynomolgus and rhesus macaque. Of many mouse anti-human CD19 antibodies, BU12 can cross react with B lymphocytes derived from cynomolgus macaque with lower binding affinity (Liu et al., 2016).

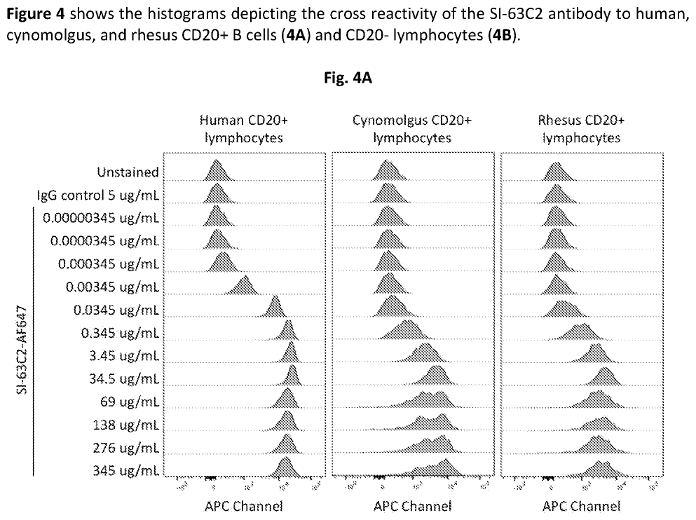

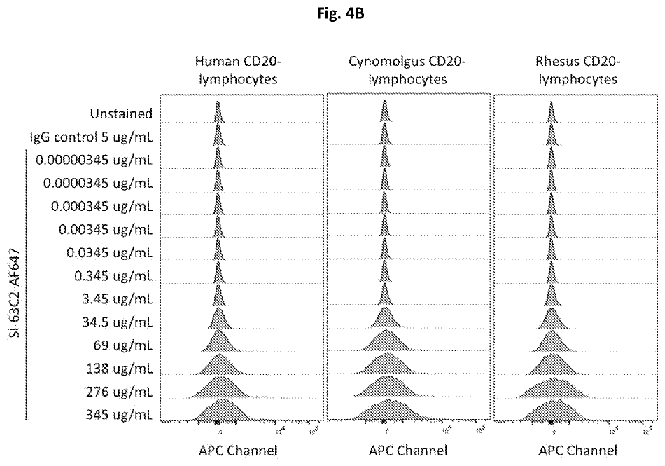

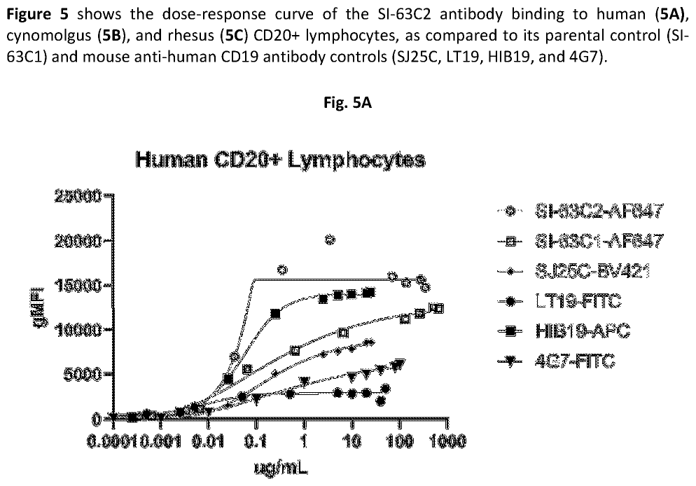

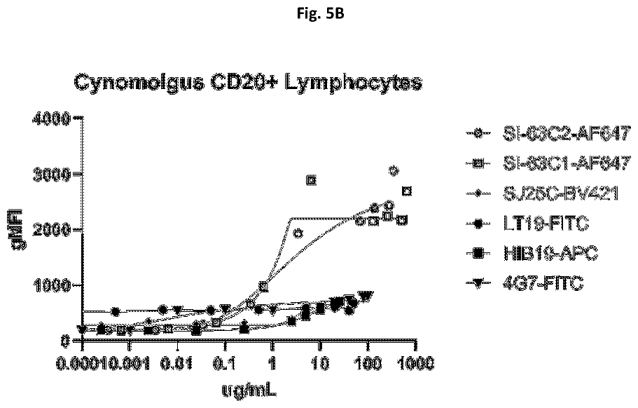

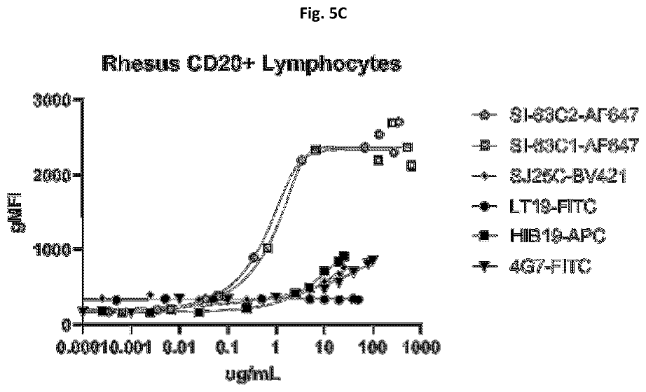

To determine if the humanization alters the cross reactivity, the flow cytometry was carried out. The SI-63C2 antibody was used to bind the peripheral blood mononuclear cells derived from human, cynomolgus, and rhesus, respectively. Lymphocytes were gated based on forward and side scatter, followed by single cells based on the ratio of forward scatter signal height and area. Viable CD20+ B-cell and CD20− lymphocytes are gated based on the exclusion of membrane permeable amine reactive dye and the binding level of CD20 antibody (clone 2H7, Biolegend). Binding of the labelled antibody was determined as the geometric mean fluorescence intensity (gMFI) of the cell population for the fluorescent conjugate's emission channel. As shown in the histogram analysis in FIG. 4, the SI-63C2 antibody binds to CD20+ B cells from human, cynomolgus, and rhesus (4A) but not to their CD20− lymphocytes (4B). When a panel of anti-CD19 antibodies (namely, SJ25C, LT19, HIB19, and 4G7) were used for comparison, only SI-63C2 and its the parental antibody, SI-63C1, displayed significant binding affinity to CD20+ B cells from human, cynomolgus, and rhesus (FIG. 5). These data confirmed the binding specificity of SI-63C2 to human, cynomolgus, and rhesus B cells was retained, however, its cross reactivity to cynomolgus, as measured by EC50, remains lower than its response to human CD19 (Table 3).

### Example 5. His-Tagged Humanized Anti-CD19 scFv Proteins

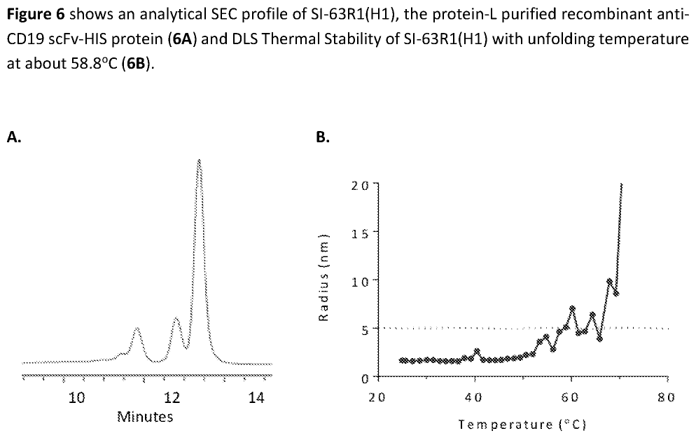

To characterize humanized anti-CD19 binding domain as a scFv unit, the DNA sequences encoding humanized anti-CD19 variable regions (H1) were cloned into a His-tagged scFv expression vector containing the residues GSHHHHHH at the C-terminal of the scFv (SEQ ID NO. 41). Using the ExpiCHO expression system, the humanized anti-CD19 scFv-His-tagged protein was expressed, purified via protein L affinity chromatography, and named as SI-63R1. The data from analytical SEC indicated that SI-63R1 had 70% protein of interest, and DLS thermal stability test measured the unfolding temperature for SI-63R1 at 58.8° C. (FIG. 6).

To assess the binding affinity of SI-63R1, Octet binding assay was used. The SI-63R1 protein was loaded via covalent coupling onto AR2G sensors at 10 ug/ml and bound to a serial dilution of His-tagged human CD19 (1:2.5 dilutions from the highest concentration of 200 nM). The result shows that SI-63R1 has a binding affinity to human CD19 in the low nanomolar range (Table 2).

### Example 6. Humanized Anti-CD19 scFv monoFc Fusion Proteins

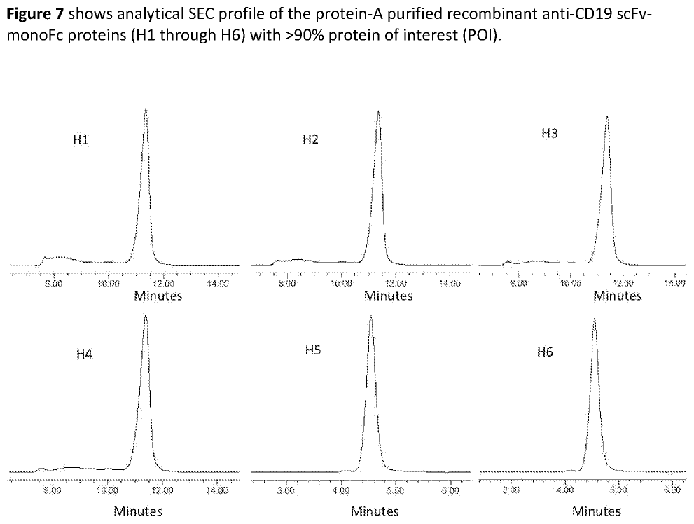

To further screen and compare all humanized peptides, the DNA sequences encoding humanized CD19 binding variants (H1, H2, H3, H4, H5 and H6) were configured to a scFv-monoFc format and cloned (Dimitrov et al. 2012.) (SEQ ID NO. 55,57,59,61,63,65). Using the ExpiCHO expression system, each of 6 humanized anti-CD19 scFv monoFc fusion proteins was expressed and purified via protein-A affinity chromatography. They were given names as SI-63SF1(H1), SI-63SF2(H2), SI-63SF4(H3), SI-63SF5(H4), SI-63SF6(H5), and SI-63SF7(H6). Following the expression and purification processes, all six proteins were characterized for their physical characters, including yields (titer), purity (% HMW and aSEC), binding affinity (KD, Kon, and Kdis) to human CD19, and thermal stability. For Octet assay, the scFv-monoFc fusion proteins were loaded via AHC sensors at 10 ug/ml and bound to a serial dilution of His-tagged human CD19 (1:2.5 dilutions starting from the highest concentration of 200 nM), and the resulting global fit to a 1:1 binding model. For the DLS analysis, the temperature was ramped from 25° C. to 75° C. at 0.5° C./min while the radius of the scFv-monoFc fusion proteins (at 1 mg/ml) was monitored by a Wyatt DynaPro Plate Reader III. The analytical SEC profiles are shown in FIG. 7, and all the measurements are listed in Table 4.

The data revealed that SI-63SF5 (H4) has the highest DLS melting temperature (Tm) at 51.8° C. (Table 4). Due to its higher thermal stability, humanized anti-CD19 variable region with H4 peptide was selected for further investigation in the GNC antibody platform.

### Example 7. Humanized Anti-CD19 scFv or Fab Domain in GNC Antibodies

The Guidance and Navigation Control (GNC) antibodies refer to a multi-specific antibody capable of binding to antigen(s) expressed by at least one target cell (including but not limited to a tumor cell, an immune cell, or a microbial cell) and the antigen expressed by at least one effector cell (such as immune cell) (see Applicant's application WO/2019/005642, incorporated herein in its entirety). A GNC antibody comprises an antibody structure of Fab and Fc regions with various additional binding domains attached to the antibody-core, such as one or more single chain fragment variable domains, also known as scFv. GNC antibodies are capable of targeting tumor antigens, engaging immune-activating receptors, and directing immune effector cell-mediated killing of tumors at a fraction of the cost. For example, it has been shown that tetra-specific GNC (tetra-GNC) antibodies exert desirable multi-facet effects with structurally and functionally diverse but relatively independent binding domains (see Applicant's application WO/2019/191120, incorporated herein in its entirety). In this context, the humanized anti-CD19 variable domain may be added to any GNC antibody as either a Fab or scFv domain.

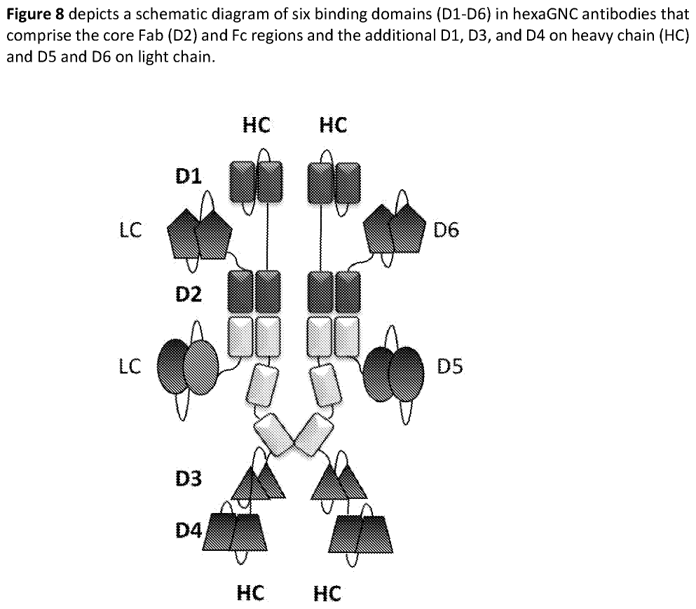

To characterize the humanized CD19 binding domain in GNC antibodies, the DNA sequences encoding H4 and H7 were configured and cloned into the GNC antibody format in one of five scFv positions and the Fab position, respectively (FIG. 8 shows the configuration scheme). A mutation R19S (Kabat numbering) was optionally incorporated into the FR1 region of the humanized (H4) VH domain for VH3-containing scFvs on the GNC light chain, e.g. SI-55H11. When scFvs containing VH3 are attached to the GNC light chain, the V_{H} domain can bind to protein A resin during purification, causing formation of light chain monomers and dimers to contaminate the desired heavy-light chain heterotetramer. In order to rationally disrupt protein A binding of VH3 family members, a structural approach was taken to interrupt the binding interface. Crystal structure 1DEE (Graille M. et al. Proc. Nat. Acad. Sci. 2000.) showed that residue R19 in VH3 (Kabat numbering) is in direct contact with two side chains of protein A domain D. In particular, contact with Q32 and D36 could be eliminated to significantly weaken the interaction. Thus, R19 was mutated to serine, which does not form these interactions due to its shorter side-chain. Additionally, S19 exists naturally in other VH family members, suggesting that it may be less immunogenic than other substitutions. For hexaGNC antibodies, which may contain up to two VH3 scFvs per chain, this mutation is especially important in allowing efficient purification of the desired product.

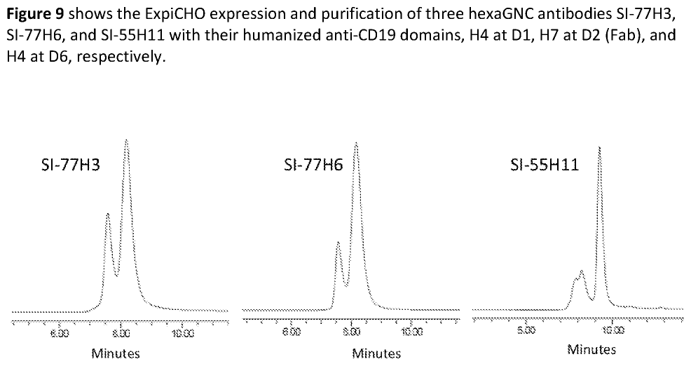

Table 5 listed the hexaGNC antibodies having a humanized CD19 binding domain H4 at D1 of SI-77H3 (SEQ ID NO. 67 and 69), at D2 (Fab) of SI-77H6 (SEQ ID NO. 71, 73), and at D6 of SI-55H11 (SEQ ID NO. 75 and 77); and the pentaGNC antibody having a humanized CD19 binding domain H4 at D6 of SI-38P12 (SEQ ID NO. 87 and 89). The expression vectors encoding these GNC antibodies were transfected and expressed in the ExpiCHO system and all GNC antibodies were purified via protein-A affinity chromatography. The results of yields and purity as measured by titer and aSEC demonstrated that the GNC antibodies with a humanized CD19 binding domain, as either a scFv or a Fab, can be expressed and purified (FIG. 9 and Table 6).

To determine the binding affinity of the hexa and pentaGNC antibodies to human CD19, the Octet binding assay was used. The GNC antibodies were loaded via AHC sensors at 10 ug/ml and bound to a serial dilution (1:2.5 dilutions starting from the highest concentration of 200 nM) or a single 100-nM concentration of His-tagged human CD19. The resulting global fit to a 1:1 binding model demonstrated that these GNC antibodies bind to CD19 with affinities in the low nanomolar range (Table 6).

### Example 8. The Positional Effect of a Humanized CD19 Binding Domain in GNC Antibodies

To evaluate the humanized CD19 binding domain mediated antibody-dependent cellular cytotoxicity, peripheral blood mononuclear cells (PBMCs) from human and cynomolgus macaque were used. T cell engagers were added to human or cynomolgus PBMC and cultured for 5 days. After 5 days, the culture cells are collected, and both viable and non-viable CD20+ B cell were counted by FACS. Analyses of both viable single B cells and viable all B cells (singlets, doublets, or other cells in the gate) were independently evaluated. Relative total cell counts are quantified using spiked in counting bead controls. In this study, the hexaGNC antibodies being tested included SI-77H3 (H4 at D1), SI-77H6 (H7 at D2, i.e. Fab), SI-55H11 (H4 at D6), and the control was a tetraGNC antibody, SI-38E17 (SEQ ID NO. 79 and 81), which has a human CD19 binding domain (21D4) at the Fab region (D2) (Table 5).

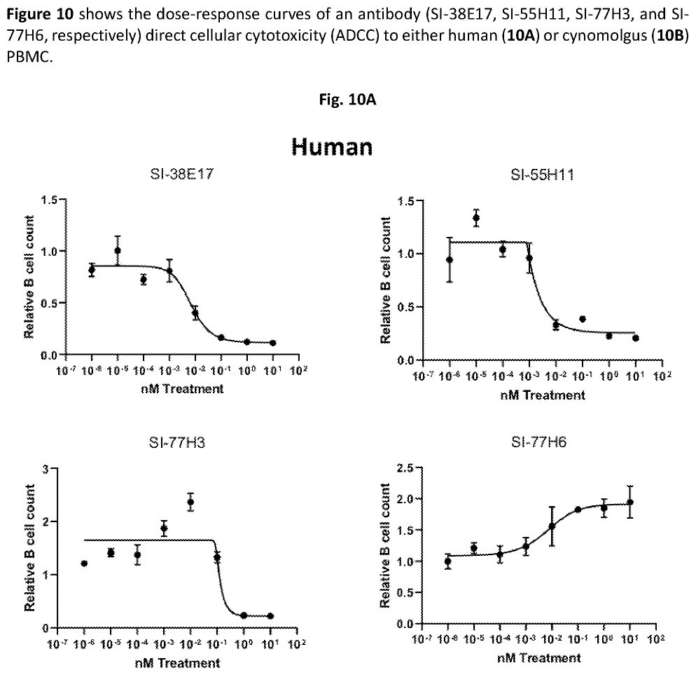

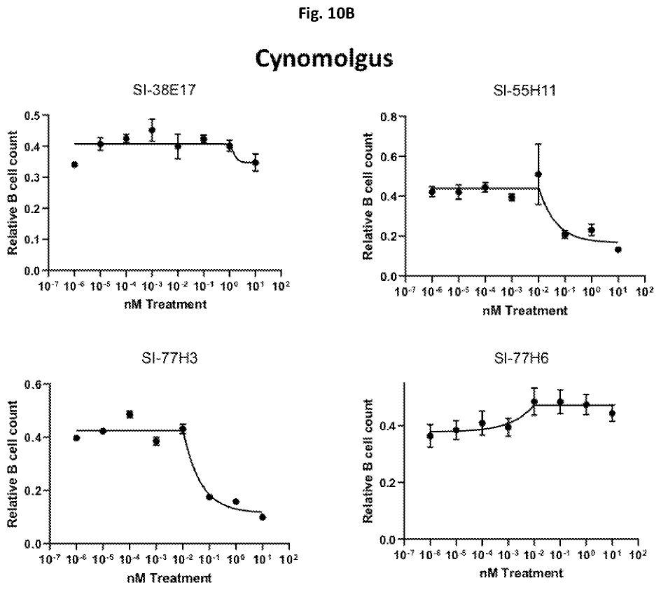

The single cell analysis by FACS tends to miss the effect of T cell engagers on the formation of non-cytolytic complexes, most of which seem to fall outside the gate for single cells. In contrast, the analysis that is inclusive of doublet cells covers more events, thereby provides more complete understanding of cell-cell interactions. FIG. 10 shows the results of ADCC analyses using the gate on viable all B cells. The control antibody, SI-38E17, displayed the binding specificity to human CD19 but not to cynomolgus CD19. As a comparison, all three hexaGNC antibodies showed similar responses to both human and cynomolgus PBMC. Unexpectedly, SI-77H6 seemed not to mediate the ADCC to both human and cynomolgus PBMC despite the presence of a humanized CD19 binding affinity (Table 6). As shown in Table 5, in SI-77H6, the humanized CD19 binding domain is the Fab region of the antibody-core structure, whereas in both SI-77H3 and SI-55H11, the humanized CD19 binding domain is an added scFv domain to the antibody-core structure. A hexa-GNC antibody possesses at least 6 binding specificities, thereby is capable of binding at least two different types of cells in vivo and at the same time, which is a different situation from assessing the affinity of individual binding domains. This observed correlation of position and effect suggested that there are biologically distinct outcomes mediated by the positional effect of the humanized anti-CD19 Fab domain in a GNC antibody, and that SI-77H6 may not be able to support the proper formation of cytolytic immune synapses between activated T cells and target B cells. Since CD19 is expressed by both normal and neoplastic B cells, the positional effect may be useful when assigning each binding domain for the benefit of treating different types of cancer, i.e. solid versus liquid tumors. For example, a SI-77H6 liked GNC antibody may still be useful for treating solid tumors with higher efficacy but lower cytotoxicity to normal B cells. In another example the SI-77H6 liked GNC antibody could be useful for engagement of B cell help to T cells in cytolytic interaction directed toward other tumor associated antigens.

### Example 9. RTCC by Hexa-GNC Antibodies Having a Humanized CD19 Binding Domain

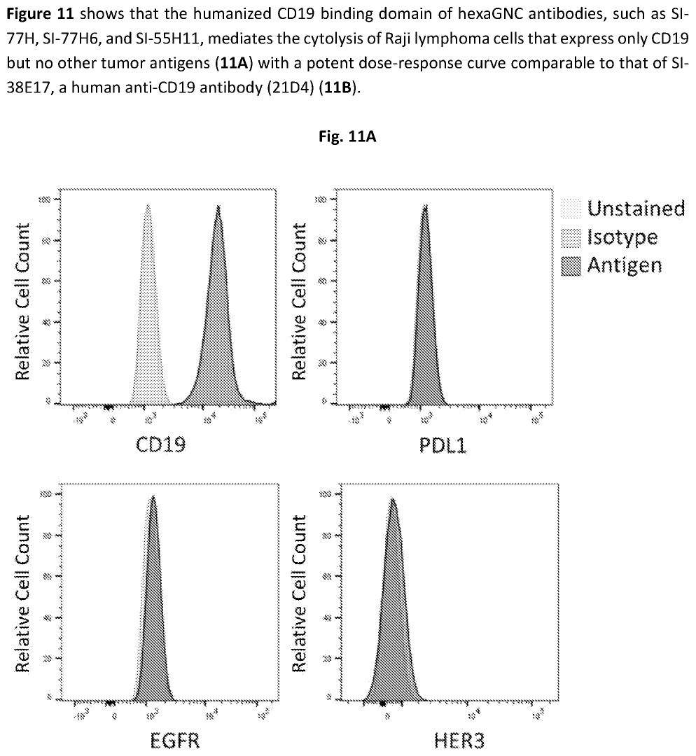

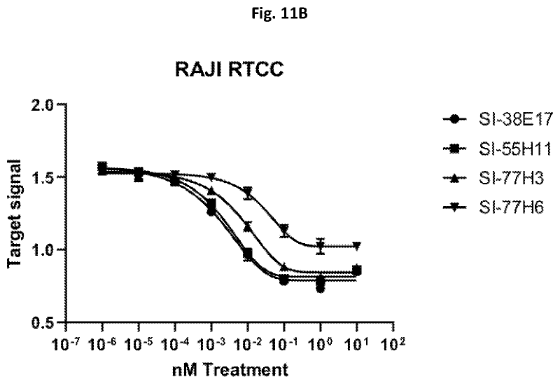

To demonstrate the cytotoxic effect of hexaGNC antibodies having a humanized CD19 binding domain, the analysis of re-directed T cell cytotoxicity (RTCC) was carried out using Raji cells. The Raji line of lymphoblast-like cells was derived from a Burkitt's lymphoma. Since each of the three hexaGNC antibodies can bind to multiple tumor antigens other than CD19, such as EGFR, HER3, and PD-L1 (Table 5), the Raji cells expressing mKate2 fluorescent protein were stained by labeled monoclonal antibodies against individual tumor antigens and analyzed by FACS. The histogram results confirmed that the Raji cells expresses CD19, and that no expression of EGFR, HER3, or PD-L1 can be detected (FIG. 11A).

The Raji cells expressing mKate2 fluorescent protein were co-cultured with human CD8 T cells at a ratio of 5 T cells per Raji cell for 81 hours in the presence of T cells engager proteins at concentrations ranging from 10 nM to 1 fM in triplicate. Target cell fluorescent signal was evaluated as a measure of specific cytolysis by quantitative microscopy and dose response curves modelled using 5 parameter asymmetric sigmoidal nonlinear regression and least squares fit method using Graphpad Prism 8. As shown in FIG. 11B, the humanized CD19 binding domain in each of SI-55H11 (EC50, 2 pM), SI-77H3 (EC50, 8 pM), and SI-77H6 (EC50, 30 pM) mediated the potent cytolysis of tumor cells, and the potency of SI-55H11 was the same as that of SI-38E17 (EC50, 2 pM), a human anti-CD19 antibody (21D4) (Table 6). SI-77H6 displayed suboptimal cytolysis with reduced potency, a phenomenon in parallel to its effect of CD19 binding to normal B cells without cytolysis induction. SI-77H3 was able to complete killing tumor cells but at a reduced EC50. Thus, with optimized configuration and treatment conditions (such as the ratio of activated T cells to target cells), the disclosed humanized CD19 binding domain may exert the same potency as the human CD19 binding domain in multi-specific GNC antibodies with an added feature of cross-reactivity to cynomolgus macaque CD19.

While the present disclosure has been described with reference to particular embodiments or examples, it may be understood that the embodiments are illustrative and that the disclosure scope is not so limited. Alternative embodiments of the present disclosure may become apparent to those having ordinary skill in the art to which the present disclosure pertains. Such alternate embodiments are considered to be encompassed within the scope of the present disclosure. Accordingly, the scope of the present disclosure is defined by the appended claims and is supported by the foregoing description. All references cited or referred to in this disclosure are hereby incorporated by reference in their entireties.

### REFERENCE

1. Watkins M P, Bartlett N L. CD19-targeted immunotherapies for treatment of patients with non-Hodgkin B-cell lymphomas. Expert Opin Investig Drugs. 2018; 27(7):601-611. doi:10.1080/13543784.2018.1492549.
2. (https://www.ncbi.nlm.nih.gov/pmc/articles/PMC5058631/.)
3. McDonagh, Charlotte et. Al. CD19 Binding agents and Uses Thereof. US 20090136526 A1.
4. Rao-Naik et. al. CD19 Antibodies and their uses. US 20090142349 A1.
5. Dimitrov et al. 2012: https://www.frontiersin.org/articles/10.3389/fimmu.2017.01545/full.
6. (Graille M. et al. Proc. Nat. Acad. Sci. 2000.)

### TABLES

TABLE 1
Computational calculation of humanized variable domains fortotal
MHCII binding scores and humanness scores using
MixMHC2pred and Z score analysis algorithm, respectively.

| | | Core | | |
|---|---|---|---|---|
| | | peptides | Humanness | Humanness |
| Name of Peptides | Sequence | (MHCII | Score (Vk) | Score (VH) |
| (VH/VL) | Type | Binding) | (Z score) | (Z score) |
| BU12 (VH/VL) | Mouse | 9 | −1.40 | −1.94 |
| H1VH-H1VL | Humanized | 5 | −0.77 | −0.03 |
| H2VH-H2VL | Humanized | 5 | −0.75 | −0.03 |
| H3VH-H3VL | Humanized | 5 | −0.77 | −0.03 |
| H4VH-H4VL | Humanized | 5 | −0.75 | −0.03 |
| H5VH-H5VL | Humanized | 9 | −0.70 | 0.58 |
| H6VH-H6VL | Humanized | 9 | −0.70 | 0.57 |
| H7VH-H7VL | Humanized | 6 | −0.56 | −0.55 |
| 21D4 | Human | 12 | 0.86 | −0.06 |

TABLE 2
The purity, binding affinity, and thermal stability of SI-63C1 (with mouse parental BU12
variable sequences), SI-63C2 (with H1 humanized anti-CD19 variable sequences), SI-34C1
(with 21D4 human anti-CD19 variable sequences), and SI-63R1
(humanized anti-CD19 scFv-His-tagged protein).

| | | αCD19 | | | | | | DLS |
|---|---|---|---|---|---|---|---|---|
| | | domain | Titer | % | KD | Kon | Kdis | Tm |
| Sample ID | Format | (VH/VL) | (ug/ml) | aSEC | (nM) | (1/ms) | (1/s) | (° C.) |
| SI-63C1 | mAb | BU12 | 250 | 98% | 3.06 | 6.81E+04 | 2.09E−04 | 75.09 |
| SI-63C2 | mAb | H1 | 235 | 99% | 3.77 | 6.08E+04 | 2.29E−04 | 75.02 |
| SI-34C1 | mAb | 21D4 | 60 | 96.45% | 1.56 | 6.44E+04 | 1.01E−04 | 69.07 |
| SI-63R1 | ScFv | H1 | 120 | 70% | 1.33 | 4.99E+04 | 9.15E−05 | 58.8 |

TABLE 3
The cross reactivity of recombinant antibodies (SI-63C1 and SI-63C2)
and mouse anti-human CD19 antibodies (SJ25C, LT19, HIB19, and 4G7)
to CD20+ lymphocytes of human, cynomolgus, and rhesus origins,
as measured by EC50.

| Anti-CD19 mAb | | Human | Cynomolgus | Rhesus |
|---|---|---|---|---|
| EC50 (ug/ml) | | CD20+ | CD20+ | CD20+ |
| SI-63C2-AF647 | Humanized | 0.03974 | 2.901 | 0.6665 |
| SI-63C1-AF647 | Mouse | 0.2314 | 0.9093 | 0.9907 |
| SJ25C-BV421 | Mouse | 0.2088 | N/A | N/A |
| LT19-FITC | Mouse | 0.008687 | N/A | N/A |
| HIB19-APC | Mouse | 0.05441 | N/A | N/A |
| 4G7-FITC | Mouse | 0.5492 | N/A | N/A |

TABLE 4
The purity, binding affinity, and thermal stability of humanized anti-CD19 scFv monoFc
fusion proteins, SI-63SF1 (H1), SI-63SF2 (H2), SI-63SF4(H3), SI-63SF5 (H4), SI-63SF6 (H5),
and SI-63SF7 (H6).

| | αCD19 | | | | | | | DLS |
|---|---|---|---|---|---|---|---|---|
| | domain | Titer | % | % | KD | Kon | Kdis | Tm |
| scFv-Fc ID | (VH/VL) | (ug/ml) | HMW | aSEC | (nM) | (1/ms) | (1/s) | (° C.) |
| SI-63SF1 | H1 | 130 | 20% | 80% | 6.89 | 4.41E+04 | 1.84E−03 | 46.9 |
| SI-63SF2 | H2 | 121 | 15% | 85% | 4.22 | 4.28E+04 | 1.81E−03 | 45.1 |
| SI-63SF4 | H3 | 174 | 12% | 89% | 4.18 | 4.56E+04 | 1.91E−03 | 51.2 |
| SI-63SF5 | H4 | 110 | 11% | 88% | 5.02 | 3.72E+04 | 1.87E−03 | 51.8 |
| SI-63SF6 | H5 | 101 | 8% | 92% | 5.68 | 5.97E+04 | 3.39E−04 | 49.8 |
| SI-63SF7 | H6 | 97 | 7% | 91% | 5.37 | 7.02E+04 | 3.77E−04 | 50.1 |

TABLE 5
The positions of the
humanized CD19 binding domain and other antigen binding domains in GNC antibodies.

| | Multi- | | D2 | | | | |
|---|---|---|---|---|---|---|---|
| GNC Ab ID | Specificity | D1 | (Fab) | D3 | D4 | D5 | D6 |
| SI-77H3 | Hexa | H4 | αEGFR | αPD-Ll | α4-1BB | αHER3 | αCD3 |
| SI-77H6 | Hexa | αHER3 | H7 | αPD-Ll | α4-1BB | αHER3 | αCD3 |
| SI-55H11 | Hexa | αEGFR | αCD3 | αPD-Ll | α4-1BB | αHER3 | H4 |
| SI-38P12 | Penta | αCD20 | αCD3 | αPD-Ll | α4-1BB | — | H4 |
| SI-38E17 | Tetra | αCD3 | 21D4 | αPD-Ll | α4-1BB | — | — |

TABLE 6
The physical and functional characters of the GNC antibodies having a humanized anti-
CD19 scFv domain or Fab region.

| | | | αCD19 | | | | |
|---|---|---|---|---|---|---|---|
| | Multi- | αCD19 | domain | Titer | aSEC | αCD19 | EC50 |
| GNC Ab ID | Specificity | Position | (VH/VL) | (μg/ml) | % POI | KD (nM) | (pM) |
| SI-77H3 | Hexa | DI | H4 | 68.4 | 86.91 | 7.53 | 8.1 |
| SI-77H6 | Hexa | D2 | H4 | 58.6 | 78.51 | 5.25 | 30.2 |
| SI-55H11 | Hexa | D6 | H4 | 61.9 | 76.95 | 4.54 | 2.5 |
| SI-38P12 | Penta | D6 | H4 | 101.2 | 79.05 | 1.11 | n/a |
| SI-38E17 | Tetra | D2 | 21D4 | 61.1 | 80.54 | 1.48 | 2.1 |

SEQUENCE LISTING

| | | SEQ ID NO. | |
|---|---|---|---|
| Sample ID | Annotation | Protein | DNA |
| H1VH | Humanized version 1 variable heavy chain | 1 | 2 |
| H2VH | Humanized version 2 variable heavy chain | 3 | 4 |
| H3VH | Humanized version 3 variable heavy chain | 5 | 6 |
| H4VH | Humanized version 4 variable heavy chain | 7 | 8 |
| H5VH | Humanized version 5 variable heavy chain | 9 | 10 |
| H6VH | Humanized version 6 variable heavy chain | 11 | 12 |
| H7VH | Humanized version 7 variable heavy chain | 91 | 92 |
| H1VL | Humanized version 1 variable light chain | 13 | 14 |
| H2VL | Humanized version 2 variable light chain | 15 | 16 |
| H3VL | Humanized version 3 variable light chain | 17 | 18 |
| H4VL | Humanized version 4 variable light chain | 19 | 20 |
| H5VL | Humanized version 5 variable light chain | 21 | 22 |
| H6VL | Humanized version 6 variable light chain | 23 | 24 |
| H7VL | Humanized version 7 variable light chain | 93 | 94 |
| BU12VH | Mouse anti-CD19 heavy chain variable sequence | 25 | 26 |
| BU12VL | Mouse anti-CD19 light chain variable sequence | 27 | 28 |
| 21D4VH | Human anti-CD19 heavy chain variable sequence | 29 | 30 |
| 21D4VL | Human anti-CD19 light chain variable sequence | 31 | 32 |
| SI-63C1HC | BU12 version mAb heavy chain | 33 | 34 |
| SI-63C1LC | BU12 version mAb kappa light chain | 35 | 36 |
| SI-63C2HC | Humanized version 1 mAb heavy chain | 37 | 38 |
| SI-63C2LC | Humanized version 1 mAb light chain | 39 | 40 |
| SI-63R1 | Humanized version 1 ScFv His-tagged | 41 | 42 |
| SI-63SV1 | Humanized version 1 ScFv | 43 | 44 |
| SI-63SV2 | Humanized version 2 ScFv | 45 | 46 |
| SI-63SV3 | Humanized version 3 ScFv | 47 | 48 |
| SI-63SV4 | Humanized version 4 ScFv | 49 | 50 |
| SI-63SV5 | Humanized version 5 ScFv | 51 | 52 |
| SI-63SV6 | Humanized Version 6 ScFv | 53 | 54 |
| SI-63SV7 | Humanized Version 7 ScFv | 95 | 96 |
| SI-63SF1 | Humanized version 1 ScFv monoFc | 55 | 56 |
| SI-63SF2 | Humanized version 2 ScFv monoFc | 57 | 58 |
| SI-63SF4 | Humanized version 3 ScFv monoFc | 59 | 60 |
| SI-63SF5 | Humanized version 4 ScFv monoFc | 61 | 62 |
| SI-63SF6 | Humanized version 5 ScFv monoFc | 63 | 64 |
| SI-63SF7 | Humanized version 6 ScFv mono Fc | 65 | 66 |
| SI-77H3HC | hexaGNC heavy chain | 67 | 68 |
| SI-77H3LC | hexaGNC light chin | 69 | 70 |
| SI-77H6HC | hexaGNC heavy chain | 71 | 72 |
| SI-77H6LC | hexaGNC Light Chain | 73 | 74 |
| SI-55H11HC | hexaGNC Heavy Chain | 75 | 76 |
| SI-55H11LC | hexaGNC light chain | 77 | 78 |
| SI-38E17HC | tetraGNC Heavy chain | 79 | 80 |
| SI-38E17LC | tetraGNC Light Chain | 81 | 82 |
| SI-34C1 | 21D4 mAb Heavy Chain | 83 | 84 |
| SI-34C1 | 21D4 mAb Light Chain | 85 | 86 |
| SI-38P12 LC | pentaGNC Light Chain | 87 | 88 |
| SI-38P12 HC | pentaGNC Heavy Chain | 89 | 90 |

>Sequence ID 1: Humanized Version 1 (H1VH) Amino Acid Sequence
QVTLKESGPGLVQPGQTLRLTCAFSGFSLSTSGMGVGWIRQPPGKGLEWLAHIWWDDDKRYNPALKSRLTISKDTSKNQVYLQMNS
LDAEDTAVYYCARMELWSYYFDYWGQGTLVTVSS
>Sequence ID 2: Humanized Version 1 (H1VH) Nucleotide Sequence
CAGGTCACATTGAAGGAATCTGGCCCCGGCCTTGTTCAGCCAGGACAGACCCTTAGGCTCACCTGTGCCTTCAGTGGTTTTTCTCT
TAGCACTAGCGGTATGGGGGTCGGCTGGATTCGGCAGCCTCCCGGCAAAGGTCTTGAGTGGTTGGCTCACATTTGGTGGGACGACG
ACAAACGGTATAATCCTGCCTTGAAAAGTCGGCTGACCATTAGTAAGGATACCTCAAAAAATCAAGTGTACTTGCAAATGAATAGC
CTTGACGCCGAGGATACGGCTGTATATTATTGCGCGCGGATGGAACTCTGGTCTTACTACTTTGATTATTGGGGGCAGGGGACTCT
CGTCACGGTCTCGAGT
>Sequence ID 3: Humanized Version 2 (H2VH) Amino Acid Sequence
QVTLKESGPGLVQPGQTLRLTCAFSGFSLSTSGMGVGWIRQPPGKGLEWLAHIWWDDDKRYNPALKSRLTISKDTSKNQVYLQMNS
LDAEDTAVYYCARMELWSYYFDYWGQGTLVTVSS
>Sequence ID 4: Humanized Version 2 (H2VH) Nucleotide Sequence
CAGGTCACATTGAAGGAATCTGGCCCCGGCCTTGTTCAGCCAGGACAGACCCTTAGGCTCACCTGTGCCTTCAGTGGTTTTTCTCT
TAGCACTAGCGGTATGGGGGTCGGCTGGATTCGGCAGCCTCCCGGCAAAGGTCTTGAGTGGTTGGCTCACATTTGGTGGGACGACG
ACAAACGGTATAATCCTGCCTTGAAAAGTCGGCTGACCATTAGTAAGGATACCTCAAAAAATCAAGTGTACTTGCAAATGAATAGC
CTTGACGCCGAGGATACGGCTGTATATTATTGCGCGCGGATGGAACTCTGGTCTTACTACTTTGATTATTGGGGGCAGGGGACTCT
CGTCACGGTCTCGAGT
>Sequence ID 5: Humanized Version 3 (H3VH) Amino Acid Sequence
QVTLKESGPGLVQPGQTLRLTCAFSGFSLSTSGMGVGWIRQPPGKGLEWLAHIWWDDDKRYNPALKSRLTISKDTSKNQVYLQMNS
LDAEDTAVYYCARMELWSYYFDYWGQGTLVTVSS
>Sequence ID 6: Humanized Version 3 (H3VH) Nucleotide Sequence
CAGGTCACATTGAAGGAATCTGGCCCCGGCCTTGTTCAGCCAGGACAGACCCTTAGGCTCACCTGTGCCTTCAGTGGTTTTTCTCT
TAGCACTAGCGGTATGGGGGTCGGCTGGATTCGGCAGCCTCCCGGCAAAGGTCTTGAGTGGTTGGCTCACATTTGGTGGGACGACG
ACAAACGGTATAATCCTGCCTTGAAAAGTCGGCTGACCATTAGTAAGGATACCTCAAAAAATCAAGTGTACTTGCAAATGAATAGC
CTTGACGCCGAGGATACGGCTGTATATTATTGCGCGCGGATGGAACTCTGGTCTTACTACTTTGATTATTGGGGGCAGGGGACTCT
CGTCACGGTCTCGAGT
Sequence ID 7: Humanized Version 4 (H4VH) Amino Acid Sequence
QVTLKESGPGLVQPGQTLRLTCAFSGFSLSTSGMGVGWIRQPPGKGLEWLAHIWWDDDKRYNPALKSRLTISKDTSKNQVYLQMNS
LDAEDTAVYYCARMELWSYYFDYWGQGTLVTVSS
>Sequence ID 8: Humanized Version 4(H4VH) Nucleotide Sequence
CAGGTCACATTGAAGGAATCTGGCCCCGGCCTTGTTCAGCCAGGACAGACCCTTAGGCTCACCTGTGCCTTCAGTGGTTTTTCTCT
TAGCACTAGCGGTATGGGGGTCGGCTGGATTCGGCAGCCTCCCGGCAAAGGTCTTGAGTGGTTGGCTCACATTTGGTGGGACGACG
ACAAACGGTATAATCCTGCCTTGAAAAGTCGGCTGACCATTAGTAAGGATACCTCAAAAAATCAAGTGTACTTGCAAATGAATAGC
CTTGACGCCGAGGATACGGCTGTATATTATTGCGCGCGGATGGAACTCTGGTCTTACTACTTTGATTATTGGGGGCAGGGGACTCT
CGTCACGGTCTCGAGT
>Sequence ID 9: Humanized Version 5 (H5VH) Amino Acid Sequence
EVQLVESGGGLVQPGGSLRLSCVFSGFSLSTSGMGVGWVRQAPGKGLEWVGHIWWDDDKRYNPALKSRFTISRDTSKNTVYLQMNS
LRAEDTAVYYCARMELWSYYFDYWGQGTLVTVSS
>Sequence ID 10: Humanized Version 5 (H5VH) Nucleotide Sequence
GAGGTGCAACTTGTGGAAAGCGGCGGCGGGTTGGTGCAACCTGGCGGTTCACTTCGGCTCTCATGTGTGTTCAGTGGTTTTTCCCT
TAGCACAAGCGGGATGGGTGTCGGGTGGGTCCGCCAAGCGCCTGGCAAAGGTCTGGAATGGGTTGGTCACATTTGGTGGGATGATG
ACAAAAGGTATAATCCCGCGCTGAAATCTAGATTTACTATTAGTCGGGATACGAGTAAGAACACGGTGTATCTGCAAATGAACAGT
CTCAGGGCAGAGGATACAGCGGTATATTATTGTGCTCGAATGGAGCTGTGGTCTTACTATTTTGATTACTGGGGCCAGGGCACGTT
GGTAACGGTCTCGAGT
>Sequence ID 11: Humanized Version 6 (H6VH) Amino Acid Sequence
EVQLVESGGGLVQPGGSLRLSCSFSGFSLSTSGMGVGWVRQAPGKGLEWVGHIWWDDDKRYNPALKSRFTISRDTSKNTVYLQMNS
LRAEDTAVYYCARMELWSYYFDYWGQGTLVTVSS
>Sequence ID 12: Humanized Version 6 (H6VH) Nucleotide Sequence
GAGGTGCAACTTGTGGAAAGCGGCGGCGGGTTGGTGCAACCTGGCGGTTCACTTCGGCTCTCATGTAGCTTCAGTGGTTTTTCCCT
TAGCACAAGCGGGATGGGTGTCGGGTGGGTCCGCCAAGCGCCTGGCAAAGGTCTGGAATGGGTTGGTCACATTTGGTGGGATGATG
ACAAAAGGTATAATCCCGCGCTGAAATCTAGATTTACTATTAGTCGGGATACGAGTAAGAACACGGTGTATCTGCAAATGAACAGT
CTCAGGGCAGAGGATACAGCGGTATATTATTGTGCTCGAATGGAGCTGTGGTCTTACTATTTTGATTACTGGGGCCAGGGCACGTT
GGTAACGGTCTCGAGT
>Sequence ID 13: Humanized Version 1 (H1VL) Amino Acid Sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKLEIK
>Sequence ID 14: Humanized Version 1 (H1VL) Nucleotide Sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAATTGGAGATAAAG
>Sequence ID 15: Humanized Version 2 (H2VL) Amino Acid Sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKITIL
>Sequence ID 16: Humanized Version 2 (H2VL) Nucleotide Sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAAATTACGATACTG
>Sequence ID 17: Humanized Version 3 (H3VL) Amino Acid Sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKLTVL
>Sequence ID 18: Humanized Version 3 (H3VL) Nucleotide Sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAACTTACGGTACTG
Sequence ID 19: Humanized Version 4 (H4VL) Amino Acid Sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKVTVL
>Sequence ID 20: Humanized Version 4 (H4VL) Nucleotide Sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAAGTTACGGTACTG
>Sequence ID 21: Humanized Version 5 (H5VL) Amino Acid Sequence
EIVMTQSPSTLSASVGDRVIITCSASSSVSYMHWYQQKPGKAPKLLIYDTSKLASGVPSRFSGSGSGAEFTLTISSLQPDDFATYY
CFQGSVYPFTFGQGTKLTVL
>Sequence ID 22: Humanized Version 5 (H5VL) Nucleotide Sequence
GAAATAGTGATGACGCAGTCACCTAGCACCCTTAGTGCTTCTGTAGGAGACAGGGTTATAATTACCTGCAGTGCTAGTTCCTCAGT
GTCATACATGCACTGGTATCAGCAGAAACCGGGAAAAGCTCCAAAGCTGCTTATATACGACACGTCCAAATTGGCATCAGGTGTCC
CCAGTCGATTTAGTGGCTCTGGCTCAGGGGCTGAATTTACGCTCACAATCTCCAGCCTCCAACCAGATGACTTCGCCACATACTAC
TGTTTTCAGGGCTCAGTGTATCCGTTTACTTTCGGCCAGGGGACAAAGTTGACTGTACTT
>Sequence ID 23: Humanized Version 6 (H6VL) Amino Acid Sequence
EIVMTQSPSTLSASVGDRVIITCSASSSVSYMHWYQQKPGKAPKLLIYDTSKLASGVPSRFSGSGSGAEFTLTISSLQPDDFATYY
CFQGSVYPFTFGQGTKLTVL
>Sequence ID 24: Humanized Version 6 (H6VL) Nucleotide Sequence
GAAATAGTGATGACGCAGTCACCTAGCACCCTTAGTGCTTCTGTAGGAGACAGGGTTATAATTACCTGCAGTGCTAGTTCCTCAGT
GTCATACATGCACTGGTATCAGCAGAAACCGGGAAAAGCTCCAAAGCTGCTTATATACGACACGTCCAAATTGGCATCAGGTGTCC
CCAGTCGATTTAGTGGCTCTGGCTCAGGGGCTGAATTTACGCTCACAATCTCCAGCCTCCAACCAGATGACTTCGCCACATACTAC
TGTTTTCAGGGCTCAGTGTATCCGTTTACTTTCGGCCAGGGGACAAAGTTGACTGTACTT
>Sequence ID 25: BU12 VH: Mouse anti-CD19 VH Amino Acid Sequence
QVTLKESGPGILQPSQTLSLTCSFSGFSLSTSGMGVGWIRQPSGKGLEWLAHIWWDDDKRYNPALKSRLTISKDTSSNQVFLKIAS
VDTADTAAYYCARMELWSYYFDYWGQGTTLTVSS
>Sequence ID 26: BU12 VH: Mouse anti-CD19 VH Nucleotide Sequence
CAGGTGACCCTGAAAGAAAGCGGCCCGGGCATTCTGCAGCCGAGCCAGACCCTGAGCCTGACCTGCAGCTTTAGCGGCTTTAGCCT
GAGCACCAGCGGCATGGGCGTGGGCTGGATTCGCCAGCCGAGCGGCAAAGGCCTGGAATGGCTGGCGCATATTTGGTGGGATGATG
ATAAACGCTATAACCCGGCGCTGAAAAGCCGCCTGACCATTAGCAAAGATACCAGCAGCAACCAGGTGTTTCTGAAAATTGCGAGC
GTGGATACCGCGGATACCGCGGCGTATTATTGCGCGCGCATGGAACTGTGGAGCTATTATTTTGATTATTGGGGCCAGGGCACCAC
CCTGACCGTGAGCAGC
>Sequence ID 27: BU12 VL: Mouse anti-CD19 VL Amino Acid Sequence
ENVLTQSPAIMSASPGEKVTMTCSASSSVSYMHWYQQKSSTSPKLWIYDTSKLASGVPGRFSGSGSGNSHFLTISSMEAEDVATYY
CFQGSVYPFTFGSGTKLEIK
>Sequence ID 28: BU12 VL: Mouse anti-CD19 VL Nucleotide Sequence
GAAAACGTGCTGACCCAGAGCCCGGCGATTATGAGCGCGAGCCCGGGCGAAAAAGTGACCATGACCTGCAGCGCGAGCAGCAGCGT
GAGCTATATGCATTGGTATCAGCAGAAAAGCAGCACCAGCCCGAAACTGTGGATTTATGATACCAGCAAACTGGCGAGCGGCGTGC
CGGGCCGCTTTAGCGGCAGCGGCAGCGGCAACAGCCATTTTCTGACCATTAGCAGCATGGAAGCGGAAGATGTGGCGACCTATTAT
TGCTTTCAGGGCAGCGTGTATCCGTTTACCTTTGGCAGCGGCACCAAACTGGAAATTAAA
>Sequence ID 29: 21D4 human antibody VH Amino Acid Sequence
EVQLVQSGAEVKKPGESLKISCKGSGYSFSSSWIGWVRQAPGKGLEWMGIIYPDDSDTRYSPSFQGQVTISADKSIRTAYLQWSSL
KASDTAMYYCARHVTMIWGVIIDFWGQGTLVTVS S
>Sequence ID 30: 21D4 human antibody VH Nucleotide Sequence
GAGGTGCAGCTGGTGCAGTCTGGAGCAGAGGTGAAGAAACCAGGAGAGTCTCTGAAGATCTCCTGTAAGGGTTCTGGATACAGCTT
TAGCAGTTCATGGATCGGCTGGGTGCGCCAGGCACCTGGGAAAGGCCTGGAATGGATGGGGATCATCTATCCTGATGACTCTGATA
CCAGATACAGTCCATCCTTCCAAGGCCAGGTCACCATCTCAGCCGACAAGTCCATCAGGACTGCCTACCTGCAGTGGAGTAGCCTG
AAGGCCTCGGACACCGCTATGTATTACTGTGCGAGACATGTTACTATGATTTGGGGAGTTATTATTGACTTCTGGGGCCAGGGAAC
CCTGGTCACCGTCTCCTCA
Sequence ID 31: 21D4 human antibody VL Amino Acid Sequence
AIQLTQSPSSLSASVGDRVTITCRASQGISSALAWYQQKPGKAPKLLIYDASSLESGVPSRFSGSGSGTDFTLTISSLQPEDFATY
YCQQFNSYPFTFGPGTKVDIK
>Sequence ID 32: 21D4 human antibody VL Nucleotide Sequence
GCCATCCAGTTGACCCAGTCTCCATCCTCCCTGTCTGCATCTGTAGGAGACAGAGTCACCATCACTTGCCGGGCAAGTCAGGGCAT
TAGCAGTGCTTTAGCCTGGTATCAGCAGAAACCAGGGAAAGCTCCTAAGCTCCTGATCTATGATGCCTCCAGTTTGGAAAGTGGGG
TCCCATCAAGGTTCAGCGGCAGTGGATCTGGGACAGATTTCACTCTCACCATCAGCAGCCTGCAGCCTGAAGATTTTGCAACTTAT
TACTGTCAACAGTTTAATAGTTACCCATTCACTTTCGGCCCTGGGACCAAAGTGGATATCAAA
>Sequence ID 33: SI-63C1 Heavy Chain Amino Acid Sequence
QVTLKESGPGILQPSQTLSLTCSFSGFSLSTSGMGVGWIRQPSGKGLEWLAHIWWDDDKRYNPALKSRLTISKDTSSNQVFLKIAS
VDTADTAAYYCARMELWSYYFDYWGQGTTLTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKVEPKSCDKTHTCPPCPAPPVAGPSVFLFPPKPKDTLMISRT
PEVTCVVVDVSHEDPEVKFNWYVDGVEVHNAKTKPREEQYASTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLYSKLTVDKSRWQQGNVFSCSVM
HEALHNHYTQKSLSLSPGK
>Sequence ID 34: SI-63C1 Heavy Chain Nucleotide Sequence
CAGGTGACCCTGAAAGAAAGCGGCCCGGGCATTCTGCAGCCGAGCCAGACCCTGAGCCTGACCTGCAGCTTTAGCGGCTTTAGCCT
GAGCACCAGCGGCATGGGCGTGGGCTGGATTCGCCAGCCGAGCGGCAAAGGCCTGGAATGGCTGGCGCATATTTGGTGGGATGATG
ATAAACGCTATAACCCGGCGCTGAAAAGCCGCCTGACCATTAGCAAAGATACCAGCAGCAACCAGGTGTTTCTGAAAATTGCGAGC
GTGGATACCGCGGATACCGCGGCGTATTATTGCGCGCGCATGGAACTGTGGAGCTATTATTTTGATTATTGGGGCCAGGGCACCAC
CCTGACCGTGAGCAGCGCTAGCACCAAGGGCCCATCTGTCTTCCCCCTGGCACCCTCCTCCAAGAGCACCTCTGGGGGCACAGCTG
CCCTGGGCTGCCTGGTCAAGGACTACTTCCCTGAACCTGTGACAGTGTCCTGGAACTCAGGAGCCCTGACCAGCGGCGTGCACACC
TTCCCGGCTGTCCTACAGTCCTCAGGACTCTACTCCCTCAGCAGCGTGGTGACCGTGCCCTCCAGCAGCTTGGGCACCCAGACCTA
CATCTGCAACGTGAATCACAAGCCCAGCAACACCAAGGTGGACAAGAAAGTTGAGCCCAAATCTTGTGACAAAACTCACACATGCC
CACCGTGCCCAGCACCTCCTGTAGCAGGACCGTCAGTCTTCCTCTTCCCCCCAAAACCCAAGGACACCCTCATGATCTCCCGGACC
CCTGAGGTCACATGCGTGGTGGTGGACGTGAGCCACGAAGACCCTGAGGTCAAGTTCAACTGGTACGTGGACGGCGTGGAGGTGCA
TAATGCCAAGACAAAGCCGCGGGAGGAGCAGTACGCCAGCACGTACCGTGTGGTCAGCGTCCTCACCGTCCTGCACCAGGACTGGC
TGAATGGCAAGGAGTACAAGTGCAAGGTCTCCAACAAAGCCCTCCCAGCCCCCATCGAGAAAACCATCTCCAAAGCCAAAGGGCAG
CCCCGAGAACCACAGGTGTACACCCTGCCCCCATCCCGGGATGAGCTGACCAAGAACCAGGTCAGCCTGACCTGCCTGGTCAAAGG
CTTCTATCCCAGCGACATCGCCGTGGAGTGGGAGAGCAATGGGCAGCCGGAGAACAACTACAAGACCACGCCTCCCGTGCTGGACT
CCGACGGCTCCTTCTTCCTCTACAGCAAGCTCACCGTGGACAAGAGCAGGTGGCAGCAGGGGAACGTCTTCTCATGCTCCGTGATG
CATGAGGCTCTGCACAACCACTACACACAGAAGAGCCTCTCCCTGTCTCCGGGCAAA
>Sequence ID 35: SI-63C1 Light Chain Amino Acid Sequence
ENVLTQSPAIMSASPGEKVTMTCSASSSVSYMHWYQQKSSTSPKLWIYDTSKLASGVPGRFSGSGSGNSHFLTISSMEAEDVATYY
CFQGSVYPFTFGSGTKLEIKRTVAAPSVFIFPPSDEQLKSGTASWCLLNNFYPREAKVQWKVDNALQSGNSQESVTEQDSKDSTY
SLSSTLTLSKADYEKHKVYACEVTHQGLSSPVTKSFNRGEC
>Sequence ID 36: SI-63C1 Light Chain Nucleotide Sequence
GAAAACGTGCTGACCCAGAGCCCGGCGATTATGAGCGCGAGCCCGGGCGAAAAAGTGACCATGACCTGCAGCGCGAGCAGCAGCGT
GAGCTATATGCATTGGTATCAGCAGAAAAGCAGCACCAGCCCGAAACTGTGGATTTATGATACCAGCAAACTGGCGAGCGGCGTGC
CGGGCCGCTTTAGCGGCAGCGGCAGCGGCAACAGCCATTTTCTGACCATTAGCAGCATGGAAGCGGAAGATGTGGCGACCTATTAT
TGCTTTCAGGGCAGCGTGTATCCGTTTACCTTTGGCAGCGGCACCAAACTGGAAATTAAACGTACGGTGGCTGCACCATCTGTCTT
CATCTTCCCGCCATCTGATGAGCAGTTGAAATCTGGAACTGCCTCTGTTGTGTGCCTGCTGAATAACTTCTATCCCAGAGAGGCCA
AAGTACAGTGGAAGGTGGATAACGCCCTCCAATCGGGTAACTCCCAGGAGAGTGTCACAGAGCAGGACAGCAAGGACAGCACCTAC
AGCCTCAGCAGCACCCTGACGCTGAGCAAAGCAGACTACGAGAAACACAAAGTCTACGCCTGCGAAGTCACCCATCAGGGCCTGAG
CTCGCCCGTCACAAAGAGCTTCAACAGGGGAGAGTGT
>Sequence ID 37: SI-63C2 Heavy Chain Amino Acid Sequence
QVTLKESGPGLVQPGQTLRLTCAFSGFSLSTSGMGVGWIRQPPGKGLEWLAHIWWDDDKRYNPALKSRLTISKDTSKNQVYLQMNS
LDAEDTAVYYCARMELWSYYFDYWGQGTLVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKVEPKSCDKTHTCPPCPAPPVAGPSVFLFPPKPKDTLMISRT
PEVTCVVVDVSHEDPEVKFNWYVDGVEVHNAKTKPREEQYASTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLYSKLTVDKSRWQQGNVFSCSVM
HEALHNHYTQKSLSLSPGK
>Sequence ID 38: SI-63C2 Heavy Chain Nucleotide Sequence
CAGGTCACATTGAAGGAATCTGGCCCCGGCCTTGTTCAGCCAGGACAGACCCTTAGGCTCACCTGTGCCTTCAGTGGTTTTTCTCT
TAGCACTAGCGGTATGGGGGTCGGCTGGATTCGGCAGCCTCCCGGCAAAGGTCTTGAGTGGTTGGCTCACATTTGGTGGGACGACG
ACAAACGGTATAATCCTGCCTTGAAAAGTCGGCTGACCATTAGTAAGGATACCTCAAAAAATCAAGTGTACTTGCAAATGAATAGC
CTTGACGCCGAGGATACGGCTGTATATTATTGCGCGCGGATGGAACTCTGGTCTTACTACTTTGATTATTGGGGGCAGGGGACTCT
CGTCACGGTGTCCTCTGCTAGCACCAAGGGCCCATCTGTCTTCCCCCTGGCACCCTCCTCCAAGAGCACCTCTGGGGGCACAGCTG
CCCTGGGCTGCCTGGTCAAGGACTACTTCCCTGAACCTGTGACAGTGTCCTGGAACTCAGGAGCCCTGACCAGCGGCGTGCACACC
TTCCCGGCTGTCCTACAGTCCTCAGGACTCTACTCCCTCAGCAGCGTGGTGACCGTGCCCTCCAGCAGCTTGGGCACCCAGACCTA
CATCTGCAACGTGAATCACAAGCCCAGCAACACCAAGGTGGACAAGAAAGTTGAGCCCAAATCTTGTGACAAAACTCACACATGCC
CACCGTGCCCAGCACCTCCTGTAGCAGGACCGTCAGTCTTCCTCTTCCCCCCAAAACCCAAGGACACCCTCATGATCTCCCGGACC
CCTGAGGTCACATGCGTGGTGGTGGACGTGAGCCACGAAGACCCTGAGGTCAAGTTCAACTGGTACGTGGACGGCGTGGAGGTGCA
TAATGCCAAGACAAAGCCGCGGGAGGAGCAGTACGCCAGCACGTACCGTGTGGTCAGCGTCCTCACCGTCCTGCACCAGGACTGGC
TGAATGGCAAGGAGTACAAGTGCAAGGTCTCCAACAAAGCCCTCCCAGCCCCCATCGAGAAAACCATCTCCAAAGCCAAAGGGCAG
CCCCGAGAACCACAGGTGTACACCCTGCCCCCATCCCGGGATGAGCTGACCAAGAACCAGGTCAGCCTGACCTGCCTGGTCAAAGG
CTTCTATCCCAGCGACATCGCCGTGGAGTGGGAGAGCAATGGGCAGCCGGAGAACAACTACAAGACCACGCCTCCCGTGCTGGACT
CCGACGGCTCCTTCTTCCTCTACAGCAAGCTCACCGTGGACAAGAGCAGGTGGCAGCAGGGGAACGTCTTCTCATGCTCCGTGATG
CATGAGGCTCTGCACAACCACTACACACAGAAGAGCCTCTCCCTGTCTCCGGGCAAA
Sequence ID 39: SI-63C2 Light Chain Amino Acid Sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKLEIKRTVAAPSVFIFPPSDEQLKSGTASWCLLNNFYPREAKVQWKVDNALQSGNSQESVTEQDSKDSTY
SLSSTLTLSKADYEKHKVYACEVTHQGLSSPVTKSFNRGEC
Sequence ID 40: SI-63C2 Light Chain Nucleotide Sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAATTGGAGATAAAGCGTACGGTGGCTGCACCATCTGTCTT
CATCTTCCCGCCATCTGATGAGCAGTTGAAATCTGGAACTGCCTCTGTTGTGTGCCTGCTGAATAACTTCTATCCCAGAGAGGCCA
AAGTACAGTGGAAGGTGGATAACGCCCTCCAATCGGGTAACTCCCAGGAGAGTGTCACAGAGCAGGACAGCAAGGACAGCACCTAC
AGCCTCAGCAGCACCCTGACGCTGAGCAAAGCAGACTACGAGAAACACAAAGTCTACGCCTGCGAAGTCACCCATCAGGGCCTGAG
CTCGCCCGTCACAAAGAGCTTCAACAGGGGAGAGTGT
>Sequence ID 41: SI-63R1 (H1 ScFv-His) Amino Acid Sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKLEIKGGGGSGGGGSGGGGSGGGGSQVTLKESGPGLVQPGQTLRLTCAFSGFSLSTSGMGVGWIRQPPGKG
LEWLAHIWWDDDKRYNPALKSRLTISKDTSKNQVYLQMNSLDAEDTAVYYCARMELWSYYFDYWGQGTLVTVSSGSHHHHHH
>Sequence ID 42: SI-63R1 (H1 ScFv-His) Nucleotide Sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAATTGGAGATAAAGGGCGGTGGCGGTAGTGGGGGAGGCGG
TTCTGGCGGCGGAGGGTCCGGCGGTGGAGGATCACAGGTCACATTGAAGGAATCTGGCCCCGGCCTTGTTCAGCCAGGACAGACCC
TTAGGCTCACCTGTGCCTTCAGTGGTTTTTCTCTTAGCACTAGCGGTATGGGGGTCGGCTGGATTCGGCAGCCTCCCGGCAAAGGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCGCGGATGGAACTCTGGT
CTTACTACTTTGATTATTGGGGGCAGGGGACTCTCGTCACGGTCTCGAGTGGATCCCATCATCACCATCACCATTGA
>Sequence ID 43: SI-63SV1 Amino Acid Sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKLEIKGGGGSGGGGSGGGGSGGGGSQVTLKESGPGLVQPGQTLRLTCAFSGFSLSTSGMGVGWIRQPPGKG
LEWLAHIWWDDDKRYNPALKSRLTISKDTSKNQVYLQMNSLDAEDTAVYYCARMELWSYYFDYWGQGTLVTVSS
>Sequence ID 44: SI-63SV1 Nucleic Acid Sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAATTGGAGATAAAGGGCGGTGGCGGTAGTGGGGGAGGCGG
TTCTGGCGGCGGAGGGTCCGGCGGTGGAGGATCACAGGTCACATTGAAGGAATCTGGCCCCGGCCTTGTTCAGCCAGGACAGACCC
TTAGGCTCACCTGTGCCTTCAGTGGTTTTTCTCTTAGCACTAGCGGTATGGGGGTCGGCTGGATTCGGCAGCCTCCCGGCAAAGGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCGCGGATGGAACTCTGGT
CTTACTACTTTGATTATTGGGGGCAGGGGACTCTCGTCACGGTCTCGAGT
Sequence ID 45: SI-63SV2 Amino Acid Sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKITILGGGGSGGGGSGGGGSGGGGSQVTLKESGPGLVQPGQTLRLTCAFSGFSLSTSGMGVGWIRQPPGKG
LEWLAHIWWDDDKRYNPALKSRLTISKDTSKNQVYLQMNSLDAEDTAVYYCARMELWSYYFDYWGQGTLVTVSS
Sequence ID 46: SI-63SV2 Nucleic Acid Sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAAATTACGATACTGGGCGGTGGCGGTAGTGGGGGAGGCGG
TTCTGGCGGCGGAGGGTCCGGCGGTGGAGGATCACAGGTCACATTGAAGGAATCTGGCCCCGGCCTTGTTCAGCCAGGACAGACCC
TTAGGCTCACCTGTGCCTTCAGTGGTTTTTCTCTTAGCACTAGCGGTATGGGGGTCGGCTGGATTCGGCAGCCTCCCGGCAAAGGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCGCGGATGGAACTCTGGT
CTTACTACTTTGATTATTGGGGGCAGGGGACTCTCGTCACGGTCTCGAGT
>Sequence ID 47: SI-63SV3 Amino Acid Sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKLTVLGGGGSGGGGSGGGGSGGGGSQVTLKESGPGLVQPGQTLRLTCAFSGFSLSTSGMGVGWIRQPPGKG
LEWLAHIWWDDDKRYNPALKSRLTISKDTSKNQVYLQMNSLDAEDTAVYYCARMELWSYYFDYWGQGTLVTVSS
>Sequence ID 48: SI-63SV3 Nucleic Acid Sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAATTGACGGTACTGGGCGGTGGCGGTAGTGGGGGAGGCGG
TTCTGGCGGCGGAGGGTCCGGCGGTGGAGGATCACAGGTCACATTGAAGGAATCTGGCCCCGGCCTTGTTCAGCCAGGACAGACCC
TTAGGCTCACCTGTGCCTTCAGTGGTTTTTCTCTTAGCACTAGCGGTATGGGGGTCGGCTGGATTCGGCAGCCTCCCGGCAAAGGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCGCGGATGGAACTCTGGT
CTTACTACTTTGATTATTGGGGGCAGGGGACTCTCGTCACGGTCTCGAGT
>Sequence ID 49: SI-63SV4 Amino Acid Sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKVTVLGGGGSGGGGSGGGGSGGGGSQVTLKESGPGLVQPGQTLRLTCAFSGFSLSTSGMGVGWIRQPPGKG
LEWLAHIWWDDDKRYNPALKSRLTISKDTSKNQVYLQMNSLDAEDTAVYYCARMELWSYYFDYWGQGTLVTVSS
>Sequence ID 50: SI-63SV4 Nucleic Acid Sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAAGTTACGGTACTGGGCGGTGGCGGTAGTGGGGGAGGCGG
TTCTGGCGGCGGAGGGTCCGGCGGTGGAGGATCACAGGTCACATTGAAGGAATCTGGCCCCGGCCTTGTTCAGCCAGGACAGACCC
TTAGGCTCACCTGTGCCTTCAGTGGTTTTTCTCTTAGCACTAGCGGTATGGGGGTCGGCTGGATTCGGCAGCCTCCCGGCAAAGGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCGCGGATGGAACTCTGGT
CTTACTACTTTGATTATTGGGGGCAGGGGACTCTCGTCACGGTCTCGAGT
>Sequence ID 51: SI-63SV5 Amino Acid Sequence
EIVMTQSPSTLSASVGDRVIITCSASSSVSYMHWYQQKPGKAPKLLIYDTSKLASGVPSRFSGSGSGAEFTLTISSLQPDDFATYY
CFQGSVYPFTFGQGTKLTVLGGGGSGGGGSGGGGSGGGGSEVQLVESGGGLVQPGGSLRLSCVFSGFSLSTSGMGVGWVRQAPGKG
LEWVGHIWWDDDKRYNPALKSRFTISRDTSKNTVYLQMNSLRAEDTAVYYCARMELWSYYFDYWGQGTLVTVSS
>Sequence ID 52: SI-63SV5 Nucleic Acid Sequence
GAAATAGTGATGACGCAGTCACCTAGCACCCTTAGTGCTTCTGTAGGAGACAGGGTTATAATTACCTGCAGTGCTAGTTCCTCAGT
GTCATACATGCACTGGTATCAGCAGAAACCGGGAAAAGCTCCAAAGCTGCTTATATACGACACGTCCAAATTGGCATCAGGTGTCC
CCAGTCGATTTAGTGGCTCTGGCTCAGGGGCTGAATTTACGCTCACAATCTCCAGCCTCCAACCAGATGACTTCGCCACATACTAC
TGTTTTCAGGGCTCAGTGTATCCGTTTACTTTCGGCCAGGGGACAAAGTTGACTGTACTTGGCGGTGGCGGTAGTGGGGGAGGCGG
TTCTGGCGGCGGAGGGTCCGGCGGTGGAGGATCAGAGGTGCAACTTGTGGAAAGCGGCGGCGGGTTGGTGCAACCTGGCGGTTCAC
TTCGGCTCTCATGTGTGTTCAGTGGTTTTTCCCTTAGCACAAGCGGGATGGGTGTCGGGTGGGTCCGCCAAGCGCCTGGCAAAGGT
CTGGAATGGGTTGGTCACATTTGGTGGGATGATGACAAAAGGTATAATCCCGCGCTGAAATCTAGATTTACTATTAGTCGGGATAC
GAGTAAGAACACGGTGTATCTGCAAATGAACAGTCTCAGGGCAGAGGATACAGCGGTATATTATTGTGCTCGAATGGAGCTGTGGT
CTTACTATTTTGATTACTGGGGCCAGGGCACGTTGGTAACGGTCTCGAGT
Sequence ID 53: SI-63SV6 Amino Acid Sequence
EIVMTQSPSTLSASVGDRVIITCSASSSVSYMHWYQQKPGKAPKLLIYDTSKLASGVPSRFSGSGSGAEFTLTISSLQPDDFATYY
CFQGSVYPFTFGQGTKLTVLGGGGSGGGGSGGGGSGGGGSEVQLVESGGGLVQPGGSLRLSCSFSGFSLSTSGMGVGWVRQAPGKG
LEWVGHIWWDDDKRYNPALKSRFTISRDTSKNTVYLQMNSLRAEDTAVYYCARMELWSYYFDYWGQGTLVTVSS
Sequence ID 54: SI-63SV6 Nucleic Acid Sequence
GAAATAGTGATGACGCAGTCACCTAGCACCCTTAGTGCTTCTGTAGGAGACAGGGTTATAATTACCTGCAGTGCTAGTTCCTCAGT
GTCATACATGCACTGGTATCAGCAGAAACCGGGAAAAGCTCCAAAGCTGCTTATATACGACACGTCCAAATTGGCATCAGGTGTCC
CCAGTCGATTTAGTGGCTCTGGCTCAGGGGCTGAATTTACGCTCACAATCTCCAGCCTCCAACCAGATGACTTCGCCACATACTAC
TGTTTTCAGGGCTCAGTGTATCCGTTTACTTTCGGCCAGGGGACAAAGTTGACTGTACTTGGCGGTGGCGGTAGTGGGGGAGGCGG
TTCTGGCGGCGGAGGGTCCGGCGGTGGAGGATCAGAGGTGCAACTTGTGGAAAGCGGCGGCGGGTTGGTGCAACCTGGCGGTTCAC
TTCGGCTCTCATGTAGCTTCAGTGGTTTTTCCCTTAGCACAAGCGGGATGGGTGTCGGGTGGGTCCGCCAAGCGCCTGGCAAAGGT
CTGGAATGGGTTGGTCACATTTGGTGGGATGATGACAAAAGGTATAATCCCGCGCTGAAATCTAGATTTACTATTAGTCGGGATAC
GAGTAAGAACACGGTGTATCTGCAAATGAACAGTCTCAGGGCAGAGGATACAGCGGTATATTATTGTGCTCGAATGGAGCTGTGGT
CTTACTATTTTGATTACTGGGGCCAGGGCACGTTGGTAACGGTCTCGAGT
>Sequence ID 55: SI-63SF1 (H1 ScFv-monoFc) Amino Acid Sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKLEIKGGGGSGGGGSGGGGSGGGGSQVTLKESGPGLVQPGQTLRLTCAFSGFSLSTSGMGVGWIRQPPGKG
LEWLAHIWWDDDKRYNPALKSRLTISKDTSKNQVYLQMNSLDAEDTAVYYCARMELWSYYFDYWGQGTLVTVSS
GGSSGSGSGSTGLVPRGSTSSSGTGTSAGTPVAGPSVFLFPPKPKDTLMISRTPEVTCVVVDVSHEDPEVKFNWYVDGVEVHNAKT
KPREEQYASTYRVVSVLTVLHQDWLNGKEYKCKVSNKALPAPIEKTISKAKGQPREPQVYTLPPSRDELTKNQVSLRCHVKGFYPS
DIAVEWESNGQPENNYKTTKPVLDSDGSFFLYSTLTVDKSRWQQGNVFSCSVLHEALHNHYTQKSLSLSPGKGSGLNDIFEAQKIE
WHE
>Sequence ID 56: SI-63SF1 (H1 ScFv-monoFc) Nucleotide Sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAATTGGAGATAAAGGGCGGTGGCGGTAGTGGGGGAGGCGG
TTCTGGCGGCGGAGGGTCCGGCGGTGGAGGATCACAGGTCACATTGAAGGAATCTGGCCCCGGCCTTGTTCAGCCAGGACAGACCC
TTAGGCTCACCTGTGCCTTCAGTGGTTTTTCTCTTAGCACTAGCGGTATGGGGGTCGGCTGGATTCGGCAGCCTCCCGGCAAAGGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCGCGGATGGAACTCTGGT
CTTACTACTTTGATTATTGGGGGCAGGGGACTCTCGTCACGGTCTCGAGT
GGGGGATCCTCTGGAAGTGGCTCCGGCAGCACTGGGCTCGTACCAAGGGGGTCCACATCCAGTAGCGGTACTGGCACATCCGCGGG
AACCCCCGTCGCTGGACCGTCAGTCTTCCTCTTCCCCCCAAAACCCAAGGACACCCTCATGATCTCCCGGACCCCTGAGGTCACAT
GCGTGGTGGTGGACGTGAGCCACGAAGACCCTGAGGTCAAGTTCAACTGGTACGTGGACGGCGTGGAGGTGCATAATGCCAAGACA
AAGCCGCGGGAGGAGCAGTACGCTAGCACGTACCGTGTGGTCAGCGTCCTCACCGTCCTGCACCAGGACTGGCTGAATGGCAAGGA
GTACAAGTGCAAGGTCTCCAACAAAGCCCTCCCAGCCCCCATCGAGAAAACCATCTCCAAAGCCAAAGGGCAGCCCCGAGAACCAC
AGGTGTACACCCTGCCCCCATCCCGGGATGAGCTGACCAAGAACCAGGTCAGCCTGAGATGCCACGTCAAAGGCTTCTATCCCAGC
GACATCGCCGTGGAGTGGGAGAGCAATGGGCAGCCGGAGAACAACTACAAGACCACGAAGCCCGTGCTGGACTCCGACGGCTCCTT
CTTCCTCTACAGCACCCTCACCGTGGACAAGAGCAGGTGGCAGCAGGGGAACGTCTTCTCATGCTCCGTGCTCCATGAGGCTCTGC
ACAACCACTACACACAGAAGAGCCTCTCCCTGTCTCCGGGCAAAGGTTCAGGCCTGAACGATATTTTTGAAGCGCAGAAAATTGAA
TGGCATGAA
>Sequence ID 57: SI-63SF2 (H2 ScFv-monoFc) Amino Acid Sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKITILGGGGSGGGGSGGGGSGGGGSQVTLKESGPGLVQPGQTLRLTCAFSGFSLSTSGMGVGWIRQPPGKG
LEWLAHIWWDDDKRYNPALKSRLTISKDTSKNQVYLQMNSLDAEDTAVYYCARMELWSYYFDYWGQGTLVTVSSGGSSGSGSGSTG
LVPRGSTSSSGTGTSAGTPVAGPSVFLFPPKPKDTLMISRTPEVTCVVVDVSHEDPEVKFNWYVDGVEVHNAKTKPREEQYASTYR
VVSVLTVLHQDWLNGKEYKCKVSNKALPAPIEKTISKAKGQPREPQVYTLPPSRDELTKNQVSLRCHVKGFYPSDIAVEWESNGQP
ENNYKTTKPVLDSDGSFFLYSTLTVDKSRWQQGNVFSCSVLHEALHNHYTQKSLSLSPGKGSGLNDIFEAQKIEWHE
>Sequence ID 58: SI-63SF2 (H2 ScFv-monoFc) Nucleotide sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAAATTACGATACTGGGCGGTGGCGGTAGTGGGGGAGGCGG
TTCTGGCGGCGGAGGGTCCGGCGGTGGAGGATCACAGGTCACATTGAAGGAATCTGGCCCCGGCCTTGTTCAGCCAGGACAGACCC
TTAGGCTCACCTGTGCCTTCAGTGGTTTTTCTCTTAGCACTAGCGGTATGGGGGTCGGCTGGATTCGGCAGCCTCCCGGCAAAGGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCGCGGATGGAACTCTGGT
CTTACTACTTTGATTATTGGGGGCAGGGGACTCTCGTCACGGTCTCGAGT
GGGGGATCCTCTGGAAGTGGCTCCGGCAGCACTGGGCTCGTACCAAGGGGGTCCACATCCAGTAGCGGTACTGGCACATCCGCGGG
AACCCCCGTCGCTGGACCGTCAGTCTTCCTCTTCCCCCCAAAACCCAAGGACACCCTCATGATCTCCCGGACCCCTGAGGTCACAT
GCGTGGTGGTGGACGTGAGCCACGAAGACCCTGAGGTCAAGTTCAACTGGTACGTGGACGGCGTGGAGGTGCATAATGCCAAGACA
AAGCCGCGGGAGGAGCAGTACGCTAGCACGTACCGTGTGGTCAGCGTCCTCACCGTCCTGCACCAGGACTGGCTGAATGGCAAGGA
GTACAAGTGCAAGGTCTCCAACAAAGCCCTCCCAGCCCCCATCGAGAAAACCATCTCCAAAGCCAAAGGGCAGCCCCGAGAACCAC
AGGTGTACACCCTGCCCCCATCCCGGGATGAGCTGACCAAGAACCAGGTCAGCCTGAGATGCCACGTCAAAGGCTTCTATCCCAGC
GACATCGCCGTGGAGTGGGAGAGCAATGGGCAGCCGGAGAACAACTACAAGACCACGAAGCCCGTGCTGGACTCCGACGGCTCCTT
CTTCCTCTACAGCACCCTCACCGTGGACAAGAGCAGGTGGCAGCAGGGGAACGTCTTCTCATGCTCCGTGCTCCATGAGGCTCTGC
ACAACCACTACACACAGAAGAGCCTCTCCCTGTCTCCGGGCAAAGGTTCAGGCCTGAACGATATTTTTGAAGCGCAGAAAATTGAA
TGGCATGAA
Sequence ID 59: SI-63SF4 (H3 ScFv-monoFc) Amino Acid Sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKLTVLGGGGSGGGGSGGGGSGGGGSQVTLKESGPGLVQPGQTLRLTCAFSGFSLSTSGMGVGWIRQPPGKG
LEWLAHIWWDDDKRYNPALKSRLTISKDTSKNQVYLQMNSLDAEDTAVYYCARMELWSYYFDYWGQGTLVTVSSGGSSGSGSGSTG
LVPRGSTSSSGTGTSAGTPVAGPSVFLFPPKPKDTLMISRTPEVTCVVVDVSHEDPEVKFNWYVDGVEVHNAKTKPREEQYASTYR
VVSVLTVLHQDWLNGKEYKCKVSNKALPAPIEKTISKAKGQPREPQVYTLPPSRDELTKNQVSLRCHVKGFYPSDIAVEWESNGQP
ENNYKTTKPVLDSDGSFFLYSTLTVDKSRWQQGNVFSCSVLHEALHNHYTQKSLSLSPGKGSGLNDIFEAQKIEWHE
Sequence ID 60: SI-63SF4 (H3 ScFv-monoFc) Nucleotide Sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAACTTACGGTACTGGGCGGTGGCGGTAGTGGGGGAGGCGG
TTCTGGCGGCGGAGGGTCCGGCGGTGGAGGATCACAGGTCACATTGAAGGAATCTGGCCCCGGCCTTGTTCAGCCAGGACAGACCC
TTAGGCTCACCTGTGCCTTCAGTGGTTTTTCTCTTAGCACTAGCGGTATGGGGGTCGGCTGGATTCGGCAGCCTCCCGGCAAAGGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCGCGGATGGAACTCTGGT
CTTACTACTTTGATTATTGGGGGCAGGGGACTCTCGTCACGGTCTCGAGT
GGGGGATCCTCTGGAAGTGGCTCCGGCAGCACTGGGCTCGTACCAAGGGGGTCCACATCCAGTAGCGGTACTGGCACATCCGCGGG
AACCCCCGTCGCTGGACCGTCAGTCTTCCTCTTCCCCCCAAAACCCAAGGACACCCTCATGATCTCCCGGACCCCTGAGGTCACAT
GCGTGGTGGTGGACGTGAGCCACGAAGACCCTGAGGTCAAGTTCAACTGGTACGTGGACGGCGTGGAGGTGCATAATGCCAAGACA
AAGCCGCGGGAGGAGCAGTACGCTAGCACGTACCGTGTGGTCAGCGTCCTCACCGTCCTGCACCAGGACTGGCTGAATGGCAAGGA
GTACAAGTGCAAGGTCTCCAACAAAGCCCTCCCAGCCCCCATCGAGAAAACCATCTCCAAAGCCAAAGGGCAGCCCCGAGAACCAC
AGGTGTACACCCTGCCCCCATCCCGGGATGAGCTGACCAAGAACCAGGTCAGCCTGAGATGCCACGTCAAAGGCTTCTATCCCAGC
GACATCGCCGTGGAGTGGGAGAGCAATGGGCAGCCGGAGAACAACTACAAGACCACGAAGCCCGTGCTGGACTCCGACGGCTCCTT
CTTCCTCTACAGCACCCTCACCGTGGACAAGAGCAGGTGGCAGCAGGGGAACGTCTTCTCATGCTCCGTGCTCCATGAGGCTCTGC
ACAACCACTACACACAGAAGAGCCTCTCCCTGTCTCCGGGCAAAGGTTCAGGCCTGAACGATATTTTTGAAGCGCAGAAAATTGAA
TGGCATGAA
>Sequence ID 61: SI-63SF5 (H4 ScFv-monoFc) Amino Acid Sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKVTVLGGGGSGGGGSGGGGSGGGGSQVTLKESGPGLVQPGQTLRLTCAFSGFSLSTSGMGVGWIRQPPGKG
LEWLAHIWWDDDKRYNPALKSRLTISKDTSKNQVYLQMNSLDAEDTAVYYCARMELWSYYFDYWGQGTLVTVSSGGSSGSGSGSTG
LVPRGSTSSSGTGTSAGTPVAGPSVFLFPPKPKDTLMISRTPEVTCVVVDVSHEDPEVKFNWYVDGVEVHNAKTKPREEQYASTYR
VVSVLTVLHQDWLNGKEYKCKVSNKALPAPIEKTISKAKGQPREPQVYTLPPSRDELTKNQVSLRCHVKGFYPSDIAVEWESNGQP
ENNYKTTKPVLDSDGSFFLYSTLTVDKSRWQQGNVFSCSVLHEALHNHYTQKSLSLSPGKGSGLNDIFEAQKIEWHE
>Sequence ID 62: SI-63SF5 (H4 ScFv-monoFc) Nucleotide Sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAAGTTACGGTACTGGGCGGTGGCGGTAGTGGGGGAGGCGG
TTCTGGCGGCGGAGGGTCCGGCGGTGGAGGATCACAGGTCACATTGAAGGAATCTGGCCCCGGCCTTGTTCAGCCAGGACAGACCC
TTAGGCTCACCTGTGCCTTCAGTGGTTTTTCTCTTAGCACTAGCGGTATGGGGGTCGGCTGGATTCGGCAGCCTCCCGGCAAAGGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCGCGGATGGAACTCTGGT
CTTACTACTTTGATTATTGGGGGCAGGGGACTCTCGTCACGGTCTCGAGT
GGGGGATCCTCTGGAAGTGGCTCCGGCAGCACTGGGCTCGTACCAAGGGGGTCCACATCCAGTAGCGGTACTGGCACATCCGCGGG
AACCCCCGTCGCTGGACCGTCAGTCTTCCTCTTCCCCCCAAAACCCAAGGACACCCTCATGATCTCCCGGACCCCTGAGGTCACAT
GCGTGGTGGTGGACGTGAGCCACGAAGACCCTGAGGTCAAGTTCAACTGGTACGTGGACGGCGTGGAGGTGCATAATGCCAAGACA
AAGCCGCGGGAGGAGCAGTACGCTAGCACGTACCGTGTGGTCAGCGTCCTCACCGTCCTGCACCAGGACTGGCTGAATGGCAAGGA
GTACAAGTGCAAGGTCTCCAACAAAGCCCTCCCAGCCCCCATCGAGAAAACCATCTCCAAAGCCAAAGGGCAGCCCCGAGAACCAC
AGGTGTACACCCTGCCCCCATCCCGGGATGAGCTGACCAAGAACCAGGTCAGCCTGAGATGCCACGTCAAAGGCTTCTATCCCAGC
GACATCGCCGTGGAGTGGGAGAGCAATGGGCAGCCGGAGAACAACTACAAGACCACGAAGCCCGTGCTGGACTCCGACGGCTCCTT
CTTCCTCTACAGCACCCTCACCGTGGACAAGAGCAGGTGGCAGCAGGGGAACGTCTTCTCATGCTCCGTGCTCCATGAGGCTCTGC
ACAACCACTACACACAGAAGAGCCTCTCCCTGTCTCCGGGCAAAGGTTCAGGCCTGAACGATATTTTTGAAGCGCAGAAAATTGAA
TGGCATGAA
Sequence ID 63: SI-63SF6 (H5 ScFv-monoFc) Amino Acid Sequence
EIVMTQSPSTLSASVGDRVIITCSASSSVSYMHWYQQKPGKAPKLLIYDTSKLASGVPSRFSGSGSGAEFTLTISSLQPDDFATYY
CFQGSVYPFTFGQGTKLTVLGGGGSGGGGSGGGGSGGGGSEVQLVESGGGLVQPGGSLRLSCVFSGFSLSTSGMGVGWVRQAPGKG
LEWVGHIWWDDDKRYNPALKSRFTISRDTSKNTVYLQMNSLRAEDTAVYYCARMELWSYYFDYWGQGTLVTVSSGSSGSGSGSTGL
VPRGSTSSSGTGTSAGTPVAGPSVFLFPPKPKDTLMISRTPEVTCVVVDVSHEDPEVKFNWYVDGVEVHNAKTKPREEQYASTYRV
VSVLTVLHQDWLNGKEYKCKVSNKALPAPIEKTISKAKGQPREPQVYTLPPSRDELTKNQVSLRCHVKGFYPSDIAVEWESNGQPE
NNYKTTKPVLDSDGSFFLYSTLTVDKSRWQQGNVFSCSVLHEALHNHYTQKSLSLSPGKGSGLNDIFEAQKIEWHE
Sequence ID 64: SI-63SF6 (H5 ScFv-monoFc) Nucleotide Sequence
GAAATAGTGATGACGCAGTCACCTAGCACCCTTAGTGCTTCTGTAGGAGACAGGGTTATAATTACCTGCAGTGCTAGTTCCTCAGT
GTCATACATGCACTGGTATCAGCAGAAACCGGGAAAAGCTCCAAAGCTGCTTATATACGACACGTCCAAATTGGCATCAGGTGTCC
CCAGTCGATTTAGTGGCTCTGGCTCAGGGGCTGAATTTACGCTCACAATCTCCAGCCTCCAACCAGATGACTTCGCCACATACTAC
TGTTTTCAGGGCTCAGTGTATCCGTTTACTTTCGGCCAGGGGACAAAGTTGACTGTACTTGGCGGTGGCGGTAGTGGGGGAGGCGG
TTCTGGCGGCGGAGGGTCCGGCGGTGGAGGATCAGAGGTGCAACTTGTGGAAAGCGGCGGCGGGTTGGTGCAACCTGGCGGTTCAC
TTCGGCTCTCATGTGTGTTCAGTGGTTTTTCCCTTAGCACAAGCGGGATGGGTGTCGGGTGGGTCCGCCAAGCGCCTGGCAAAGGT
CTGGAATGGGTTGGTCACATTTGGTGGGATGATGACAAAAGGTATAATCCCGCGCTGAAATCTAGATTTACTATTAGTCGGGATAC
GAGTAAGAACACGGTGTATCTGCAAATGAACAGTCTCAGGGCAGAGGATACAGCGGTATATTATTGTGCTCGAATGGAGCTGTGGT
CTTACTATTTTGATTACTGGGGCCAGGGCACGTTGGTAACGGTCTCGAGTGGATCCTCTGGAAGTGGCTCCGGCAGCACTGGGCTC
GTACCAAGGGGGTCCACATCCAGTAGCGGTACTGGCACATCCGCGGGAACCCCCGTCGCTGGACCGTCAGTCTTCCTCTTCCCCCC
AAAACCCAAGGACACCCTCATGATCTCCCGGACCCCTGAGGTCACATGCGTGGTGGTGGACGTGAGCCACGAAGACCCTGAGGTCA
AGTTCAACTGGTACGTGGACGGCGTGGAGGTGCATAATGCCAAGACAAAGCCGCGGGAGGAGCAGTACGCTAGCACGTACCGTGTG
GTCAGCGTCCTCACCGTCCTGCACCAGGACTGGCTGAATGGCAAGGAGTACAAGTGCAAGGTCTCCAACAAAGCCCTCCCAGCCCC
CATCGAGAAAACCATCTCCAAAGCCAAAGGGCAGCCCCGAGAACCACAGGTGTACACCCTGCCCCCATCCCGGGATGAGCTGACCA
AGAACCAGGTCAGCCTGAGATGCCACGTCAAAGGCTTCTATCCCAGCGACATCGCCGTGGAGTGGGAGAGCAATGGGCAGCCGGAG
AACAACTACAAGACCACGAAGCCCGTGCTGGACTCCGACGGCTCCTTCTTCCTCTACAGCACCCTCACCGTGGACAAGAGCAGGTG
GCAGCAGGGGAACGTCTTCTCATGCTCCGTGCTCCATGAGGCTCTGCACAACCACTACACACAGAAGAGCCTCTCCCTGTCTCCGG
GCAAAGGTTCAGGCCTGAACGATATTTTTGAAGCGCAGAAAATTGAATGGCATGAA
>Sequence ID 65: SI-63SF7 (H6 ScFv-monoFc) Amino Acid Sequence
EIVMTQSPSTLSASVGDRVIITCSASSSVSYMHWYQQKPGKAPKLLIYDTSKLASGVPSRFSGSGSGAEFTLTISSLQPDDFATYY
CFQGSVYPFTFGQGTKLTVLGGGGSGGGGSGGGGSGGGGSEVQLVESGGGLVQPGGSLRLSCSFSGFSLSTSGMGVGWVRQAPGKG
LEWVGHIWWDDDKRYNPALKSRFTISRDTSKNTVYLQMNSLRAEDTAVYYCARMELWSYYFDYWGQGTLVTVSSGSSGSGSGSTGL
VPRGSTSSSGTGTSAGTPVAGPSVFLFPPKPKDTLMISRTPEVTCVVVDVSHEDPEVKFNWYVDGVEVHNAKTKPREEQYASTYRV
VSVLTVLHQDWLNGKEYKCKVSNKALPAPIEKTISKAKGQPREPQVYTLPPSRDELTKNQVSLRCHVKGFYPSDIAVEWESNGQPE
NNYKTTKPVLDSDGSFFLYSTLTVDKSRWQQGNVFSCSVLHEALHNHYTQKSLSLSPGKGSGLNDIFEAQKIEWHE
>Sequence ID 66: SI-63SF7 (H6 ScFv-monoFc) Nucleotide Sequence
GAAATAGTGATGACGCAGTCACCTAGCACCCTTAGTGCTTCTGTAGGAGACAGGGTTATAATTACCTGCAGTGCTAGTTCCTCAGT
GTCATACATGCACTGGTATCAGCAGAAACCGGGAAAAGCTCCAAAGCTGCTTATATACGACACGTCCAAATTGGCATCAGGTGTCC
CCAGTCGATTTAGTGGCTCTGGCTCAGGGGCTGAATTTACGCTCACAATCTCCAGCCTCCAACCAGATGACTTCGCCACATACTAC
TGTTTTCAGGGCTCAGTGTATCCGTTTACTTTCGGCCAGGGGACAAAGTTGACTGTACTTGGCGGTGGCGGTAGTGGGGGAGGCGG
TTCTGGCGGCGGAGGGTCCGGCGGTGGAGGATCAGAGGTGCAACTTGTGGAAAGCGGCGGCGGGTTGGTGCAACCTGGCGGTTCAC
TTCGGCTCTCATGTAGCTTCAGTGGTTTTTCCCTTAGCACAAGCGGGATGGGTGTCGGGTGGGTCCGCCAAGCGCCTGGCAAAGGT
CTGGAATGGGTTGGTCACATTTGGTGGGATGATGACAAAAGGTATAATCCCGCGCTGAAATCTAGATTTACTATTAGTCGGGATAC
GAGTAAGAACACGGTGTATCTGCAAATGAACAGTCTCAGGGCAGAGGATACAGCGGTATATTATTGTGCTCGAATGGAGCTGTGGT
CTTACTATTTTGATTACTGGGGCCAGGGCACGTTGGTAACGGTCTCGAGTGGATCCTCTGGAAGTGGCTCCGGCAGCACTGGGCTC
GTACCAAGGGGGTCCACATCCAGTAGCGGTACTGGCACATCCGCGGGAACCCCCGTCGCTGGACCGTCAGTCTTCCTCTTCCCCCC
AAAACCCAAGGACACCCTCATGATCTCCCGGACCCCTGAGGTCACATGCGTGGTGGTGGACGTGAGCCACGAAGACCCTGAGGTCA
AGTTCAACTGGTACGTGGACGGCGTGGAGGTGCATAATGCCAAGACAAAGCCGCGGGAGGAGCAGTACGCTAGCACGTACCGTGTG
GTCAGCGTCCTCACCGTCCTGCACCAGGACTGGCTGAATGGCAAGGAGTACAAGTGCAAGGTCTCCAACAAAGCCCTCCCAGCCCC
CATCGAGAAAACCATCTCCAAAGCCAAAGGGCAGCCCCGAGAACCACAGGTGTACACCCTGCCCCCATCCCGGGATGAGCTGACCA
AGAACCAGGTCAGCCTGAGATGCCACGTCAAAGGCTTCTATCCCAGCGACATCGCCGTGGAGTGGGAGAGCAATGGGCAGCCGGAG
AACAACTACAAGACCACGAAGCCCGTGCTGGACTCCGACGGCTCCTTCTTCCTCTACAGCACCCTCACCGTGGACAAGAGCAGGTG
GCAGCAGGGGAACGTCTTCTCATGCTCCGTGCTCCATGAGGCTCTGCACAACCACTACACACAGAAGAGCCTCTCCCTGTCTCCGG
GCAAAGGTTCAGGCCTGAACGATATTTTTGAAGCGCAGAAAATTGAATGGCATGAA
Sequence ID 67: SI-77H3 Heavy Chain Amino Acid Sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKVTVLGGGGSGGGGSGGGGSGGGGSQVTLKESGPGLVQPGQTLSLTCAFSGFSLSTSGMGVGWIRQPPGKG
LEWLAHIWWDDDKRYNPALKSRLTISKDTSKNQVYLQMNSLDAEDTAVYYCARMELWSYYFDYWGQGTLVTVSSGGGGSGGGGSQV
QLQQSGPGLVKPSETLSITCTVSGFSLTNYGVHWIRQAPGKCLEWLGVIWSGGNTDYNTPFTSRFTITKDNSKNQVYFKLRSVRAD
DTAIYYCARALTYYDYEFAYWGQGTLVTVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKRVEPKSCDKTHTCPPCPAPEAAGAPSVFLFPPKPKDTLMISRTPE
VTCVVVDVSHEDPEVKFNWYVDGVEVHNAKTKPREEQYNSTYRVVSVLTVLHQDWLNGKEYKCAVSNKALPAPIEKTISKAKGQPR
EPQVYTLPPSRDELTKNQVSLTCLVKGFYPSDIAVEWESNGQPENNYKTTPPVLDSDGSFFLYSKLTVDKSRWQQGNVFSCSVMHE
ALHNHYTQKSLSLSPGGGGGSGGGGSEVQLLESGGGLVQPGGSLRLSCAASGFSFSSGYDMCWVRQAPGKGLEWIACIAAGSAGIT
YDANWAKGRFTISRDNSKNTLYLQMNSLRAEDTAVYYCARSAFSFDYAMDLWGQGTLVTVSSGGGGSGGGGSGGGGSGGGGSDIQM
TQSPSTLSASVGDRVTITCQASQSISSHLNWYQQKPGKAPKLLIYKASTLASGVPSRFSGSGSGTEFTLTISSLQPDDFATYYCQQ
GYSWGNVDNVFGGGTKVEIKGGGGSGGGGSGRSLVESGGGLVQPGGSLRLSCTASGFTISSYHMQWVRQAPGKGLEYIGTISSGGN
VYYASSARGRFTISRPSSKNTVDLQMNSLRAEDTAVYYCARDSGYSDPMWGQGTLVTVSSGGGGSGGGGSGGGGSGGGGSDVVMTQ
SPSSVSASVGDRVTITCQASQNIRTYLSWYQQKPGKAPKLLIYAAANLASGVPSRFSGSGSGTDFTLTISDLEPGDAATYYCQSTY
LGTDYVGGAFGGGTKVEIK
>Sequence ID 68: SI-77H3 Heavy Chain Nucleic Acid Sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAAGTGACCGTCCTAGGCGGTGGCGGTAGTGGGGGAGGCGG
TTCTGGCGGCGGAGGGTCCGGCGGTGGAGGATCACAGGTCACATTGAAGGAATCTGGCCCCGGCCTTGTTCAGCCAGGACAGACCC
TTAGCCTCACCTGTGCCTTCAGTGGTTTTTCTCTTAGCACTAGCGGTATGGGGGTCGGCTGGATTCGGCAGCCTCCCGGCAAAGGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCTCGGATGGAACTCTGGT
CTTACTACTTTGATTATTGGGGGCAGGGGACTCTCGTCACGGTCTCGAGTGGCGGTGGAGGGTCCGGCGGTGGTGGATCACAAGTA
CAGTTGCAGCAATCCGGTCCCGGTCTCGTCAAACCGAGTGAGACGCTTAGTATAACGTGTACTGTTTCAGGCTTTAGCCTTACGAA
CTATGGAGTTCACTGGATTCGGCAGGCACCCGGCAAATGTTTGGAATGGCTGGGTGTTATTTGGTCAGGTGGAAATACAGACTATA
ACACCCCCTTTACAAGTCGGTTCACAATTACGAAAGATAATTCCAAAAATCAAGTTTATTTCAAGTTGAGATCCGTCCGCGCGGAC
GACACTGCGATCTACTATTGTGCGAGGGCACTGACCTACTACGATTACGAATTTGCGTATTGGGGGCAAGGGACTCTTGTAACAGT
CTCCAGTGCTAGCACCAAGGGCCCATCGGTCTTCCCCCTGGCACCCTCCTCCAAGAGCACCTCTGGGGGCACAGCGGCCCTGGGCT
GCCTGGTCAAGGACTACTTCCCCGAACCGGTGACGGTGTCGTGGAACTCAGGCGCCCTGACCAGCGGCGTGCACACCTTCCCGGCT
GTCCTACAGTCCTCAGGACTCTACTCCCTCAGCAGCGTGGTGACCGTGCCCTCCAGCAGCTTGGGCACCCAGACCTACATCTGCAA
CGTGAATCACAAGCCCAGCAACACCAAGGTGGACAAGAGAGTTGAGCCCAAATCTTGTGACAAAACTCACACATGCCCACCGTGCC
CAGCACCTGAAGCCGCGGGGGCACCGTCAGTCTTCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCAACTGGTACGTGGACGGCGTGGAGGTGCATAATGC
CAAGACAAAGCCGCGGGAGGAGCAGTACAACAGCACGTACCGTGTGGTCAGCGTCCTCACCGTCCTGCACCAGGACTGGCTGAATG
GCAAGGAGTACAAGTGCGCGGTCTCCAACAAAGCCCTCCCAGCCCCCATCGAGAAAACCATCTCCAAAGCCAAAGGGCAGCCCCGA
GAACCACAGGTGTACACCCTGCCCCCATCCCGGGATGAGCTGACCAAGAACCAGGTCAGCCTGACCTGCCTGGTCAAAGGCTTCTA
TCCCAGCGACATCGCCGTGGAGTGGGAGAGCAATGGGCAGCCGGAGAACAACTACAAGACCACGCCTCCCGTGCTGGACTCCGACG
GCTCCTTCTTCCTCTATAGCAAGCTCACCGTGGACAAGAGCAGGTGGCAGCAGGGGAACGTCTTCTCATGCTCCGTGATGCATGAG
GCTCTGCACAACCACTACACGCAGAAGAGCCTCTCCCTGTCTCCGGGTGGCGGTGGAGGGTCCGGCGGTGGTGGATCCGAGGTGCA
GCTGTTGGAGTCTGGGGGAGGCTTGGTACAGCCTGGGGGGTCCCTGAGACTCTCCTGTGCAGCCTCTGGATTCTCCTTCAGTAGCG
GGTACGACATGTGCTGGGTCCGCCAGGCTCCAGGGAAGGGGCTGGAGTGGATCGCATGCATTGCTGCTGGTAGTGCTGGTATCACT
TACGACGCGAACTGGGCGAAAGGCCGGTTCACCATCTCCAGAGACAATTCCAAGAACACGCTGTATCTGCAAATGAACAGCCTGAG
AGCCGAGGACACGGCCGTATATTACTGTGCGAGATCGGCGTTTTCGTTCGACTACGCCATGGACCTCTGGGGCCAGGGAACCCTGG
TCACCGTGTCGAGCGGTGGAGGCGGATCTGGCGGAGGTGGTTCCGGCGGTGGCGGCTCCGGTGGAGGCGGCTCTGACATCCAGATG
ACCCAGTCTCCTTCCACCCTGTCTGCATCTGTAGGAGACAGAGTCACCATCACTTGCCAGGCCAGTCAGAGCATTAGTTCCCACTT
AAACTGGTATCAGCAGAAACCAGGGAAAGCCCCTAAGCTCCTGATCTATAAGGCATCCACTCTGGCATCTGGGGTCCCATCAAGGT
TCAGCGGCAGTGGATCTGGGACAGAATTTACTCTCACCATCAGCAGCCTGCAGCCTGATGATTTTGCAACTTATTACTGCCAACAG
GGTTATAGTTGGGGTAATGTTGATAATGTTTTCGGCGGAGGGACCAAGGTGGAGATCAAAGGCGGTGGAGGGTCCGGCGGTGGTGG
CTCCGGACGGTCGCTGGTGGAGTCTGGGGGAGGCTTGGTCCAGCCTGGGGGGTCCCTGAGACTCTCCTGTACTGCCTCTGGATTCA
CCATCAGTAGCTACCACATGCAGTGGGTCCGCCAGGCTCCAGGGAAGGGGCTGGAGTACATCGGAACCATTAGTAGTGGTGGTAAT
GTATACTACGCAAGCTCCGCTAGAGGCAGATTCACCATCTCCAGACCCTCGTCCAAGAACACGGTGGATCTTCAAATGAACAGCCT
GAGAGCCGAGGACACGGCTGTGTATTACTGTGCGAGAGACTCTGGTTATAGTGATCCTATGTGGGGCCAGGGAACCCTGGTCACCG
TCTCTTCAGGCGGTGGCGGTAGTGGGGGAGGCGGTTCTGGCGGCGGAGGGTCCGGCGGTGGAGGATCAGACGTTGTGATGACCCAG
TCTCCATCTTCCGTGTCTGCATCTGTAGGAGACAGAGTCACCATCACCTGTCAGGCCAGTCAGAACATTAGGACTTACTTATCCTG
GTATCAGCAGAAACCAGGGAAAGCCCCTAAGCTCCTGATCTATGCTGCAGCCAATCTGGCATCTGGGGTCCCATCAAGGTTCAGCG
GCAGTGGATCTGGGACAGATTTCACTCTCACCATCAGCGACCTGGAGCCTGGCGATGCTGCAACTTACTATTGTCAGTCTACCTAT
CTTGGTACTGATTATGTTGGCGGTGCTTTCGGCGGAGGGACCAAGGTGGAGATCAAATGA
Sequence ID 69: SI-77H3 Light Chain Amino Acid Sequence
EIVMTQSPSTLSASVGDRVIITCQASESISSWLAWYQQKPGKAPKLLIYEASKLASGVPSRFSGSGSGAEFTLTISSLQPDDFATY
YCQGYFYFISRTYVNSFGQGTKLTVLGGGGSGGGGSGGGGSGGGGSEVQLVESGGGLVQPGGSLSLSCAASGFTISTNAMSWVRQA
PGKGLEWVGVITGRDITYYASWAKGRFTISRDTSKNTVYLQMNSLRAEDTAVYYCARDGGSSAITSNNIWGQGTLVTVSSGGGGSG
GGGSEIVLTQSPSTLSVSPGERATFSCRASQSIGTNIHWYQQKPGKPPRLLIKYASESISGIPDRFSGSGSGTEFTLTISSVQSED
FAVYYCQQNNNWPTTFGCGTKLTVLRTVAAPSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGECGGGGSGGGGSGGGGSQSALTQPASVSGSPGQSITISCTGT
SSDVGGYNFVSWYQQHPGKAPKLMIYDVSDRPSGVSDRFSGSKSGNTASLIISGLQADDEADYYCSSYGSSSTHVIFGGGTKVTVL
GGGGSGGGGSGGGGSGGGGSQVQLQESGGGLVKPGGSLSLSCAASGFTFSSYWMSWVRQAPGKGLEWVANINRDGSASYYVDSVKG
RFTISRDDAKNSLYLQMNSLRAEDTAVYYCARDRGVGYFDLWGRGTLVTVSS
Sequence ID 70: SI-77H3 Light Chain Nucleotide Sequence
GAAATCGTTATGACGCAGAGTCCCTCCACGCTCTCCGCTAGTGTCGGGGATCGCGTCATTATCACATGCCAGGCCTCCGAGTCAAT
CAGCAGCTGGCTTGCATGGTATCAACAGAAGCCGGGAAAAGCTCCTAAATTGCTGATCTATGAAGCGTCAAAATTGGCGTCTGGTG
TCCCATCTAGGTTCTCCGGCTCTGGGTCTGGTGCGGAATTTACTTTGACAATCTCCAGTCTTCAACCAGACGATTTCGCTACCTAC
TACTGCCAAGGGTATTTCTATTTTATAAGCCGGACATATGTAAACTCCTTCGGCCAAGGAACAAAGTTGACTGTTCTTGGTGGCGG
AGGCAGTGGTGGCGGGGGCAGCGGAGGTGGTGGTTCAGGGGGTGGTGGGAGCGAAGTCCAATTGGTAGAAAGTGGCGGTGGTCTGG
TGCAACCTGGTGGATCTCTTAGCCTCTCATGCGCCGCTAGTGGCTTTACTATTTCAACTAATGCGATGAGCTGGGTTCGCCAGGCC
CCCGGCAAAGGACTTGAGTGGGTCGGCGTCATCACCGGCAGGGACATTACATACTATGCGAGTTGGGCAAAGGGCAGGTTCACGAT
TAGCCGCGATACTTCAAAGAATACCGTTTACCTTCAAATGAATAGCTTGAGGGCGGAAGACACAGCTGTGTATTACTGCGCGAGGG
ATGGAGGTAGTTCCGCCATAACTTCCAACAACATATGGGGACAAGGCACGCTGGTTACTGTCTCGAGTGGCGGTGGAGGGTCCGGC
GGTGGTGGATCAGAAATCGTCCTTACACAATCTCCTAGCACACTGAGTGTGAGCCCCGGCGAACGCGCGACTTTCTCTTGCAGGGC
AAGTCAATCCATAGGGACTAATATACATTGGTATCAACAAAAGCCAGGTAAACCACCCAGGCTTTTGATTAAGTATGCAAGTGAGT
CTATTTCCGGTATCCCTGACCGCTTCTCTGGATCAGGCAGTGGCACAGAGTTCACACTCACCATATCTAGTGTGCAATCAGAGGAC
TTCGCCGTGTATTACTGCCAACAGAATAATAACTGGCCGACTACCTTCGGATGCGGTACAAAGCTGACCGTTTTACGTACGGTGGC
TGCACCATCTGTCTTCATCTTCCCGCCATCTGATGAGCAGTTGAAATCTGGAACTGCCTCTGTTGTGTGCCTGCTGAATAACTTCT
ATCCCAGAGAGGCCAAAGTACAGTGGAAGGTGGATAACGCCCTCCAATCGGGTAACTCCCAGGAGAGTGTCACAGAGCAGGACAGC
AAGGACAGCACCTACAGCCTCAGCAGCACCCTGACGCTGAGCAAAGCAGACTACGAGAAACACAAAGTCTACGCCTGCGAAGTCAC
CCATCAGGGCCTGAGCTCGCCCGTCACAAAGAGCTTCAACAGGGGAGAGTGTGGCGGTGGCGGTAGCGGTGGCGGCGGAAGTGGTG
GCGGAGGATCCCAGTCTGCCCTGACTCAGCCTGCCTCCGTGTCTGGGTCTCCTGGACAGTCGATCACCATCTCCTGCACTGGAACC
AGCAGTGACGTTGGTGGTTATAACTTTGTCTCCTGGTACCAACAACACCCAGGCAAAGCCCCCAAACTCATGATCTATGATGTCAG
TGATCGGCCCTCAGGGGTGTCTGATCGCTTCTCCGGCTCCAAGTCTGGCAACACGGCCTCCCTGATCATCTCTGGCCTCCAGGCTG
ACGACGAGGCTGATTATTACTGCAGCTCATATGGGAGCAGCAGCACTCATGTGATTTTCGGCGGAGGGACCAAGGTGACCGTCCTA
GGTGGAGGCGGTTCAGGCGGAGGTGGTTCCGGCGGTGGCGGCTCCGGTGGAGGCGGCTCTCAGGTGCAATTGCAGGAGTCGGGGGG
AGGCCTGGTCAAGCCTGGAGGGTCCCTGAGTCTCTCCTGTGCAGCCTCTGGATTCACCTTTAGTAGTTATTGGATGAGCTGGGTCC
GCCAGGCTCCAGGGAAGGGGCTGGAGTGGGTGGCCAACATAAACCGCGATGGAAGTGCGAGTTACTATGTGGACTCTGTGAAGGGC
CGATTCACCATCTCCAGAGACGACGCCAAGAACTCACTGTATCTGCAAATGAACAGCCTGAGAGCTGAGGACACGGCTGTGTATTA
CTGTGCGAGAGATCGTGGGGTGGGCTACTTCGATCTCTGGGGCCGTGGCACCCTGGTCACCGTCTCTAGC
>Sequence ID 71: SI-77H6 Heavy Chain Amino Acid Sequence
EIVLTQSPSTLSVSPGERATFSCRASQSIGTNIHWYQQKPGKPPRLLIKYASESISGIPDRFSGSGSGTEFTLTISSVQSEDFAVY
YCQQNNNWPTTFGPGTKLTVLGGGGSGGGGSGGGGSGGGGSQVQLQQSGPGLVKPSETLSITCTVSGFSLTNYGVHWIRQAPGKGL
EWLGVIWSGGNTDYNTPFTSRFTITKDNSKNQVYFKLRSVRADDTAIYYCARALTYYDYEFAYWGQGTLVTVSSGGGGSGGGGSQV
TLKESGPGLVQPGQTLRLTCAFSGFSLSTSGMGVGWIRQPPGKCLEWLAHIWWDDDKRYNPALKSRLTISKDTSKNQVYLQMNSLD
AEDTAVYYCARMELWSYYFDYWGQGTLVTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKRVEPKSCDKTHTCPPCPAPEAAGAPSVFLFPPKPKDTLMISRTP
EVTCVVVDVSHEDPEVKFNWYVDGVEVHNAKTKPREEQYNSTYRVVSVLTVLHQDWLNGKEYKCAVSNKALPAPIEKTISKAKGQP
REPQVYTLPPSRDELTKNQVSLTCLVKGFYPSDIAVEWESNGQPENNYKTTPPVLDSDGSFFLYSKLTVDKSRWQQGNVFSCSVMH
EALHNHYTQKSLSLSPGGGGGSGGGGSEVQLLESGGGLVQPGGSLRLSCAASGFSFSSGYDMCWVRQAPGKGLEWIACIAAGSAGI
TYDANWAKGRFTISRDNSKNTLYLQMNSLRAEDTAVYYCARSAFSFDYAMDLWGQGTLVTVSSGGGGSGGGGSGGGGSGGGGSDIQ
MTQSPSTLSASVGDRVTITCQASQSISSHLNWYQQKPGKAPKLLIYKASTLASGVPSRFSGSGSGTEFTLTISSLQPDDFATYYCQ
QGYSWGNVDNVFGGGTKVEIKGGGGSGGGGSGRSLVESGGGLVQPGGSLRLSCTASGFTISSYHMQWVRQAPGKGLEYIGTISSGG
NVYYASSARGRFTISRPSSKNTVDLQMNSLRAEDTAVYYCARDSGYSDPMWGQGTLVTVSSGGGGSGGGGSGGGGSGGGGSDVVMT
QSPSSVSASVGDRVTITCQASQNIRTYLSWYQQKPGKAPKLLIYAAANLASGVPSRFSGSGSGTDFTLTISDLEPGDAATYYCQST
YLGTDYVGGAFGGGTKVEIK
>Sequence ID 72: SI-77H6 Heavy Chain Nucleotide Sequence
GAAATCGTCCTTACACAATCTCCTAGCACACTGAGTGTGAGCCCCGGCGAACGCGCGACTTTCTCTTGCAGGGCAAGTCAATCCAT
AGGGACTAATATACATTGGTATCAACAAAAGCCAGGTAAACCACCCAGGCTTTTGATTAAGTATGCAAGTGAGTCTATTTCCGGTA
TCCCTGACCGCTTCTCTGGATCAGGCAGTGGCACAGAGTTCACACTCACCATATCTAGTGTGCAATCAGAGGACTTCGCCGTGTAT
TACTGCCAACAGAATAATAACTGGCCGACTACCTTCGGACCCGGTACAAAGCTGACCGTTTTAGGCGGTGGCGGTAGTGGGGGAGG
CGGTTCTGGCGGCGGAGGGTCCGGCGGTGGAGGATCACAAGTACAGTTGCAGCAATCCGGTCCCGGTCTCGTCAAACCGAGTGAGA
CGCTTAGTATAACGTGTACTGTTTCAGGCTTTAGCCTTACGAACTATGGAGTTCACTGGATTCGGCAGGCACCCGGCAAAGGTTTG
GAATGGCTGGGTGTTATTTGGTCAGGTGGAAATACAGACTATAACACCCCCTTTACAAGTCGGTTCACAATTACGAAAGATAATTC
CAAAAATCAAGTTTATTTCAAGTTGAGATCCGTCCGCGCGGACGACACTGCGATCTACTATTGTGCGAGGGCACTGACCTACTACG
ATTACGAATTTGCGTATTGGGGGCAAGGGACTCTTGTAACAGTCTCGAGCGGCGGTGGAGGGTCCGGCGGTGGTGGATCACAGGTC
ACATTGAAGGAATCTGGCCCCGGCCTTGTTCAGCCAGGACAGACCCTTAGGCTCACCTGTGCCTTCAGTGGTTTTTCTCTTAGCAC
TAGCGGTATGGGGGTCGGCTGGATTCGGCAGCCTCCCGGCAAATGTCTTGAGTGGTTGGCTCACATTTGGTGGGACGACGACAAAC
GGTATAATCCTGCCTTGAAAAGTCGGCTGACCATTAGTAAGGATACCTCAAAAAATCAAGTGTACTTGCAAATGAATAGCCTTGAC
GCCGAGGATACGGCTGTATATTATTGCGCTCGGATGGAACTCTGGTCTTACTACTTTGATTATTGGGGGCAGGGGACTCTCGTCAC
GGTGTCGAGTGCTAGCACCAAGGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACCAGCGGCGTGCACACCTTCCCG
GCTGTCCTACAGTCCTCAGGACTCTACTCCCTCAGCAGCGTGGTGACCGTGCCCTCCAGCAGCTTGGGCACCCAGACCTACATCTG
CAACGTGAATCACAAGCCCAGCAACACCAAGGTGGACAAGAGAGTTGAGCCCAAATCTTGTGACAAAACTCACACATGCCCACCGT
GCCCAGCACCTGAAGCCGCGGGGGCACCGTCAGTCTTCCTCTTCCCCCCAAAACCCAAGGACACCCTCATGATCTCCCGGACCCCT
GAGGTCACATGCGTGGTGGTGGACGTGAGCCACGAAGACCCTGAGGTCAAGTTCAACTGGTACGTGGACGGCGTGGAGGTGCATAA
TGCCAAGACAAAGCCGCGGGAGGAGCAGTACAACAGCACGTACCGTGTGGTCAGCGTCCTCACCGTCCTGCACCAGGACTGGCTGA
ATGGCAAGGAGTACAAGTGCGCGGTCTCCAACAAAGCCCTCCCAGCCCCCATCGAGAAAACCATCTCCAAAGCCAAAGGGCAGCCC
CGAGAACCACAGGTGTACACCCTGCCCCCATCCCGGGATGAGCTGACCAAGAACCAGGTCAGCCTGACCTGCCTGGTCAAAGGCTT
CTATCCCAGCGACATCGCCGTGGAGTGGGAGAGCAATGGGCAGCCGGAGAACAACTACAAGACCACGCCTCCCGTGCTGGACTCCG
ACGGCTCCTTCTTCCTCTATAGCAAGCTCACCGTGGACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGGTGGCGGTGGAGGGTCCGGCGGTGGTGGATCCGAGGT
GCAGCTGTTGGAGTCTGGGGGAGGCTTGGTACAGCCTGGGGGGTCCCTGAGACTCTCCTGTGCAGCCTCTGGATTCTCCTTCAGTA
GCGGGTACGACATGTGCTGGGTCCGCCAGGCTCCAGGGAAGGGGCTGGAGTGGATCGCATGCATTGCTGCTGGTAGTGCTGGTATC
ACTTACGACGCGAACTGGGCGAAAGGCCGGTTCACCATCTCCAGAGACAATTCCAAGAACACGCTGTATCTGCAAATGAACAGCCT
GAGAGCCGAGGACACGGCCGTATATTACTGTGCGAGATCGGCGTTTTCGTTCGACTACGCCATGGACCTCTGGGGCCAGGGAACCC
TGGTCACCGTGTCGAGCGGTGGAGGCGGATCTGGCGGAGGTGGTTCCGGCGGTGGCGGCTCCGGTGGAGGCGGCTCTGACATCCAG
ATGACCCAGTCTCCTTCCACCCTGTCTGCATCTGTAGGAGACAGAGTCACCATCACTTGCCAGGCCAGTCAGAGCATTAGTTCCCA
CTTAAACTGGTATCAGCAGAAACCAGGGAAAGCCCCTAAGCTCCTGATCTATAAGGCATCCACTCTGGCATCTGGGGTCCCATCAA
GGTTCAGCGGCAGTGGATCTGGGACAGAATTTACTCTCACCATCAGCAGCCTGCAGCCTGATGATTTTGCAACTTATTACTGCCAA
CAGGGTTATAGTTGGGGTAATGTTGATAATGTTTTCGGCGGAGGGACCAAGGTGGAGATCAAAGGCGGTGGAGGGTCCGGCGGTGG
TGGCTCCGGACGGTCGCTGGTGGAGTCTGGGGGAGGCTTGGTCCAGCCTGGGGGGTCCCTGAGACTCTCCTGTACTGCCTCTGGAT
TCACCATCAGTAGCTACCACATGCAGTGGGTCCGCCAGGCTCCAGGGAAGGGGCTGGAGTACATCGGAACCATTAGTAGTGGTGGT
AATGTATACTACGCAAGCTCCGCTAGAGGCAGATTCACCATCTCCAGACCCTCGTCCAAGAACACGGTGGATCTTCAAATGAACAG
CCTGAGAGCCGAGGACACGGCTGTGTATTACTGTGCGAGAGACTCTGGTTATAGTGATCCTATGTGGGGCCAGGGAACCCTGGTCA
CCGTCTCTTCAGGCGGTGGCGGTAGTGGGGGAGGCGGTTCTGGCGGCGGAGGGTCCGGCGGTGGAGGATCAGACGTTGTGATGACC
CAGTCTCCATCTTCCGTGTCTGCATCTGTAGGAGACAGAGTCACCATCACCTGTCAGGCCAGTCAGAACATTAGGACTTACTTATC
CTGGTATCAGCAGAAACCAGGGAAAGCCCCTAAGCTCCTGATCTATGCTGCAGCCAATCTGGCATCTGGGGTCCCATCAAGGTTCA
GCGGCAGTGGATCTGGGACAGATTTCACTCTCACCATCAGCGACCTGGAGCCTGGCGATGCTGCAACTTACTATTGTCAGTCTACC
TATCTTGGTACTGATTATGTTGGCGGTGCTTTCGGCGGAGGGACCAAGGTGGAGATCAAA
Sequence ID 73: SI-77H6 Light Chain Amino Acid Sequence
EIVMTQSPSTLSASVGDRVIITCQASESISSWLAWYQQKPGKAPKLLIYEASKLASGVPSRFSGSGSGAEFTLTISSLQPDDFATY
YCQGYFYFISRTYVNSFGQGTKLTVLGGGGSGGGGSGGGGSGGGGSEVQLVESGGGLVQPGGSLSLSCAASGFTISTNAMSWVRQA
PGKGLEWVGVITGRDITYYASWAKGRFTISRDTSKNTVYLQMNSLRAEDTAVYYCARDGGSSAITSNNIWGQGTLVTVSSGGGGSG
GGGSENVLTQSPASLSASPGERVTITCSASSSVSYMHWYQQKPGQAPKLWIYDTSKLASGVPSRFSGSGSGNDHTLTISSMEPEDF
ATYYCFQGSVYPFTFGCGTKVEIKRTVAAPSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGECGGGGSGGGGSGGGGSQSALTQPASVSGSPGQSITISCTGTS
SDVGGYNFVSWYQQHPGKAPKLMIYDVSDRPSGVSDRFSGSKSGNTASLIISGLQADDEADYYCSSYGSSSTHVIFGGGTKVTVLG
GGGSGGGGSGGGGSGGGGSQVQLQESGGGLVKPGGSLSLSCAASGFTFSSYWMSWVRQAPGKGLEWVANINRDGSASYYVDSVKGR
FTISRDDAKNSLYLQMNSLRAEDTAVYYCARDRGVGYFDLWGRGTLVTVSS
>Sequence ID 74: SI-77H6 Light Chain Nucleotide sequence
GAAATCGTTATGACGCAGAGTCCCTCCACGCTCTCCGCTAGTGTCGGGGATCGCGTCATTATCACATGCCAGGCCTCCGAGTCAAT
CAGCAGCTGGCTTGCATGGTATCAACAGAAGCCGGGAAAAGCTCCTAAATTGCTGATCTATGAAGCGTCAAAATTGGCGTCTGGTG
TCCCATCTAGGTTCTCCGGCTCTGGGTCTGGTGCGGAATTTACTTTGACAATCTCCAGTCTTCAACCAGACGATTTCGCTACCTAC
TACTGCCAAGGGTATTTCTATTTTATAAGCCGGACATATGTAAACTCCTTCGGCCAAGGAACAAAGTTGACTGTTCTTGGTGGCGG
AGGCAGTGGTGGCGGGGGCAGCGGAGGTGGTGGTTCAGGGGGTGGTGGGAGCGAAGTCCAATTGGTAGAAAGTGGCGGTGGTCTGG
TGCAACCTGGTGGATCTCTTAGCCTCTCATGCGCCGCTAGTGGCTTTACTATTTCAACTAATGCGATGAGCTGGGTTCGCCAGGCC
CCCGGCAAAGGACTTGAGTGGGTCGGCGTCATCACCGGCAGGGACATTACATACTATGCGAGTTGGGCAAAGGGCAGGTTCACGAT
TAGCCGCGATACTTCAAAGAATACCGTTTACCTTCAAATGAATAGCTTGAGGGCGGAAGACACAGCTGTGTATTACTGCGCGAGGG
ATGGAGGTAGTTCCGCCATAACTTCCAACAACATATGGGGACAAGGCACGCTGGTTACTGTCTCGAGCGGCGGTGGAGGGTCCGGC
GGTGGTGGATCAGAAAATGTATTGACACAGAGCCCCGCCTCCCTCAGTGCCTCACCTGGGGAAAGGGTAACTATCACTTGCTCTGC
ATCAAGCAGCGTCTCATACATGCATTGGTATCAACAAAAGCCTGGACAGGCCCCCAAGCTCTGGATATACGATACGAGCAAGCTGG
CTTCCGGCGTACCTAGCCGCTTCAGTGGTTCCGGCTCAGGCAACGATCACACCCTTACGATTTCCAGTATGGAACCCGAAGATTTT
GCAACTTATTATTGTTTCCAGGGGAGCGTGTACCCATTCACTTTCGGGTGTGGGACAAAAGTGGAGATCAAGCGTACGGTGGCTGC
ACCATCTGTCTTCATCTTCCCGCCATCTGATGAGCAGTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGTAACTCCCAGGAGAGTGTCACAGAGCAGGACAGCAAG
GACAGCACCTACAGCCTCAGCAGCACCCTGACGCTGAGCAAAGCAGACTACGAGAAACACAAAGTCTACGCCTGCGAAGTCACCCA
TCAGGGCCTGAGCTCGCCCGTCACAAAGAGCTTCAACAGGGGAGAGTGTGGCGGTGGCGGTAGCGGTGGCGGCGGAAGTGGTGGCG
GAGGATCCCAGTCTGCCCTGACTCAGCCTGCCTCCGTGTCTGGGTCTCCTGGACAGTCGATCACCATCTCCTGCACTGGAACCAGC
AGTGACGTTGGTGGTTATAACTTTGTCTCCTGGTACCAACAACACCCAGGCAAAGCCCCCAAACTCATGATCTATGATGTCAGTGA
TCGGCCCTCAGGGGTGTCTGATCGCTTCTCCGGCTCCAAGTCTGGCAACACGGCCTCCCTGATCATCTCTGGCCTCCAGGCTGACG
ACGAGGCTGATTATTACTGCAGCTCATATGGGAGCAGCAGCACTCATGTGATTTTCGGCGGAGGGACCAAGGTGACCGTCCTAGGT
GGAGGCGGTTCAGGCGGAGGTGGTTCCGGCGGTGGCGGCTCCGGTGGAGGCGGCTCTCAGGTGCAATTGCAGGAGTCGGGGGGAGG
CCTGGTCAAGCCTGGAGGGTCCCTGAGTCTCTCCTGTGCAGCCTCTGGATTCACCTTTAGTAGTTATTGGATGAGCTGGGTCCGCC
AGGCTCCAGGGAAGGGGCTGGAGTGGGTGGCCAACATAAACCGCGATGGAAGTGCGAGTTACTATGTGGACTCTGTGAAGGGCCGA
TTCACCATCTCCAGAGACGACGCCAAGAACTCACTGTATCTGCAAATGAACAGCCTGAGAGCTGAGGACACGGCTGTGTATTACTG
TGCGAGAGATCGTGGGGTGGGCTACTTCGATCTCTGGGGCCGTGGCACCCTGGTCACCGTCTCTAGC
Sequence ID 75: SI-55H11 Heavy Chain Amino Acid Sequence
EIVLTQSPSTLSVSPGERATFSCRASQSIGTNIHWYQQKPGKPPRLLIKYASESISGIPDRFSGSGSGTEFTLTISSVQSEDFAVY
YCQQNNNWPTTFGPGTKLTVLGGGGSGGGGSGGGGSGGGGSQVQLQQSGPGLVKPSETLSITCTVSGFSLTNYGVHWIRQAPGKGL
EWLGVIWSGGNTDYNTPFTSRFTITKDNSKNQVYFKLRSVRADDTAIYYCARALTYYDYEFAYWGQGTLVTVSSGGGGSGGGGSGG
GGSGGGGSEVQLVESGGGLVQPGGSLRLSCAASGFTISTNAMSWVRQAPGKCLEWIGVITGRDITYYASWAKGRFTISRDNSKNTL
YLQMNSLRAEDTAVYYCARDGGSSAITSNNIWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKRVEPKSCDKTHTCPPCPAPEAAGAPSVFLFPPKP
KDTLMISRTPEVTCVVVDVSHEDPEVKFNWYVDGVEVHNAKTKPREEQYNSTYRVVSVLTVLHQDWLNGKEYKCAVSNKALPAPIE
KTISKAKGQPREPQVYTLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGGGGGSGGGGSEVQLLESGGGLVQPGGSLRLSCAASGFSFSSGYDMCWVRQAPGKGLEWI
ACIAAGSAGITYDANWAKGRFTISRDNSKNTLYLQMNSLRAEDTAVYYCARSAFSFDYAMDLWGQGTLVTVSSGGGGSGGGGSGGG
GSGGGGSDIQMTQSPSTLSASVGDRVTITCQASQSISSHLNWYQQKPGKAPKLLIYKASTLASGVPSRFSGSGSGTEFTLTISSLQ
PDDFATYYCQQGYSWGNVDNVFGGGTKVEIKGGGGSGGGGSGRSLVESGGGLVQPGGSLRLSCTASGFTISSYHMQWVRQAPGKGL
EYIGTISSGGNVYYASSARGRFTISRPSSKNTVDLQMNSLRAEDTAVYYCARDSGYSDPMWGQGTLVTVSSGGGGSGGGGSGGGGS
GGGGSDWMTQSPSSVSASVGDRVTITCQASQNIRTYLSWYQQKPGKAPKLLIYAAANLASGVPSRFSGSGSGTDFTLTISDLEPG
DAATYYCQSTYLGTDYVGGAFGGGTKVEIK
>Sequence ID 76: SI-55H11 Heavy Chain Nucleotide Sequence
GAAATCGTCCTTACACAATCTCCTAGCACACTGAGTGTGAGCCCCGGCGAACGCGCGACTTTCTCTTGCAGGGCAAGTCAATCCAT
AGGGACTAATATACATTGGTATCAACAAAAGCCAGGTAAACCACCCAGGCTTTTGATTAAGTATGCAAGTGAGTCTATTTCCGGTA
TCCCTGACCGCTTCTCTGGATCAGGCAGTGGCACAGAGTTCACACTCACCATATCTAGTGTGCAATCAGAGGACTTCGCCGTGTAT
TACTGCCAACAGAATAATAACTGGCCGACTACCTTCGGACCCGGTACAAAGCTGACCGTTTTAGGCGGTGGCGGTAGTGGGGGAGG
CGGTTCTGGCGGCGGAGGGTCCGGCGGTGGAGGATCACAAGTACAGTTGCAGCAATCCGGTCCCGGTCTCGTCAAACCGAGTGAGA
CGCTTAGTATAACGTGTACTGTTTCAGGCTTTAGCCTTACGAACTATGGAGTTCACTGGATTCGGCAGGCACCCGGCAAAGGTTTG
GAATGGCTGGGTGTTATTTGGTCAGGTGGAAATACAGACTATAACACCCCCTTTACAAGTCGGTTCACAATTACGAAAGATAATTC
CAAAAATCAAGTTTATTTCAAGTTGAGATCCGTCCGCGCGGACGACACTGCGATCTACTATTGTGCGAGGGCACTGACCTACTACG
ATTACGAATTTGCGTATTGGGGGCAAGGGACTCTTGTAACAGTCTCGAGCGGTGGAGGCGGATCTGGCGGAGGTGGTTCCGGCGGT
GGCGGCTCCGGTGGAGGCGGCTCTGAGGTGCAGCTGGTGGAGTCTGGGGGAGGCTTGGTCCAGCCTGGGGGGTCCCTGAGACTCTC
CTGTGCAGCCTCTGGATTCACCATCAGTACCAATGCAATGAGCTGGGTCCGCCAGGCTCCAGGGAAGTGTCTGGAGTGGATCGGAG
TCATTACTGGTCGTGATATCACATACTACGCGAGCTGGGCGAAAGGCAGATTCACCATCTCCAGAGACAATTCCAAGAACACGCTG
TATCTTCAAATGAACAGCCTGAGAGCCGAGGACACGGCTGTGTATTACTGTGCGAGAGACGGTGGTTCTTCTGCTATTACTAGTAA
CAACATTTGGGGCCAGGGAACCCTGGTCACCGTGTCCTCAGCTAGCACCAAGGGCCCATCGGTCTTCCCCCTGGCACCCTCCTCCA
AGAGCACCTCTGGGGGCACAGCGGCCCTGGGCTGCCTGGTCAAGGACTACTTCCCCGAACCGGTGACGGTGTCGTGGAACTCAGGC
GCCCTGACCAGCGGCGTGCACACCTTCCCGGCTGTCCTACAGTCCTCAGGACTCTACTCCCTCAGCAGCGTGGTGACCGTGCCCTC
CAGCAGCTTGGGCACCCAGACCTACATCTGCAACGTGAATCACAAGCCCAGCAACACCAAGGTGGACAAGAGAGTTGAGCCCAAAT
CTTGTGACAAAACTCACACATGCCCACCGTGCCCAGCACCTGAAGCCGCGGGGGCACCGTCAGTCTTCCTCTTCCCCCCAAAACCC
AAGGACACCCTCATGATCTCCCGGACCCCTGAGGTCACATGCGTGGTGGTGGACGTGAGCCACGAAGACCCTGAGGTCAAGTTCAA
CTGGTACGTGGACGGCGTGGAGGTGCATAATGCCAAGACAAAGCCGCGGGAGGAGCAGTACAACAGCACGTACCGTGTGGTCAGCG
TCCTCACCGTCCTGCACCAGGACTGGCTGAATGGCAAGGAGTACAAGTGCGCGGTCTCCAACAAAGCCCTCCCAGCCCCCATCGAG
AAAACCATCTCCAAAGCCAAAGGGCAGCCCCGAGAACCACAGGTGTACACCCTGCCCCCATCCCGGGATGAGCTGACCAAGAACCA
GGTCAGCCTGACCTGCCTGGTCAAAGGCTTCTATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTATAGCAAGCTCACCGTGGACAAGAGCAGGTGGCAGCAG
GGGAACGTCTTCTCATGCTCCGTGATGCATGAGGCTCTGCACAACCACTACACGCAGAAGAGCCTCTCCCTGTCTCCGGGTGGCGG
TGGAGGGTCCGGCGGTGGTGGATCCGAGGTGCAGCTGTTGGAGTCTGGGGGAGGCTTGGTACAGCCTGGGGGGTCCCTGAGACTCT
CCTGTGCAGCCTCTGGATTCTCCTTCAGTAGCGGGTACGACATGTGCTGGGTCCGCCAGGCTCCAGGGAAGGGGCTGGAGTGGATC
GCATGCATTGCTGCTGGTAGTGCTGGTATCACTTACGACGCGAACTGGGCGAAAGGCCGGTTCACCATCTCCAGAGACAATTCCAA
GAACACGCTGTATCTGCAAATGAACAGCCTGAGAGCCGAGGACACGGCCGTATATTACTGTGCGAGATCGGCGTTTTCGTTCGACT
ACGCCATGGACCTCTGGGGCCAGGGAACCCTGGTCACCGTGTCGAGCGGTGGAGGCGGATCTGGCGGAGGTGGTTCCGGCGGTGGC
GGCTCCGGTGGAGGCGGCTCTGACATCCAGATGACCCAGTCTCCTTCCACCCTGTCTGCATCTGTAGGAGACAGAGTCACCATCAC
TTGCCAGGCCAGTCAGAGCATTAGTTCCCACTTAAACTGGTATCAGCAGAAACCAGGGAAAGCCCCTAAGCTCCTGATCTATAAGG
CATCCACTCTGGCATCTGGGGTCCCATCAAGGTTCAGCGGCAGTGGATCTGGGACAGAATTTACTCTCACCATCAGCAGCCTGCAG
CCTGATGATTTTGCAACTTATTACTGCCAACAGGGTTATAGTTGGGGTAATGTTGATAATGTTTTCGGCGGAGGGACCAAGGTGGA
GATCAAAGGCGGTGGAGGGTCCGGCGGTGGTGGCTCCGGACGGTCGCTGGTGGAGTCTGGGGGAGGCTTGGTCCAGCCTGGGGGGT
CCCTGAGACTCTCCTGTACTGCCTCTGGATTCACCATCAGTAGCTACCACATGCAGTGGGTCCGCCAGGCTCCAGGGAAGGGGCTG
GAGTACATCGGAACCATTAGTAGTGGTGGTAATGTATACTACGCAAGCTCCGCTAGAGGCAGATTCACCATCTCCAGACCCTCGTC
CAAGAACACGGTGGATCTTCAAATGAACAGCCTGAGAGCCGAGGACACGGCTGTGTATTACTGTGCGAGAGACTCTGGTTATAGTG
ATCCTATGTGGGGCCAGGGAACCCTGGTCACCGTCTCTTCAGGCGGTGGCGGTAGTGGGGGAGGCGGTTCTGGCGGCGGAGGGTCC
GGCGGTGGAGGATCAGACGTTGTGATGACCCAGTCTCCATCTTCCGTGTCTGCATCTGTAGGAGACAGAGTCACCATCACCTGTCA
GGCCAGTCAGAACATTAGGACTTACTTATCCTGGTATCAGCAGAAACCAGGGAAAGCCCCTAAGCTCCTGATCTATGCTGCAGCCA
ATCTGGCATCTGGGGTCCCATCAAGGTTCAGCGGCAGTGGATCTGGGACAGATTTCACTCTCACCATCAGCGACCTGGAGCCTGGC
GATGCTGCAACTTACTATTGTCAGTCTACCTATCTTGGTACTGATTATGTTGGCGGTGCTTTCGGCGGAGGGACCAAGGTGGAGAT
CAAA
Sequence ID 77: SI-55H11 Light Chain Amino Acid Sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKVTVLGGGGSGGGGSGGGGSGGGGSQVTLKESGPGLVQPGQTLSLTCAFSGFSLSTSGMGVGWIRQPPGKG
LEWLAHIWWDDDKRYNPALKSRLTISKDTSKNQVYLQMNSLDAEDTAVYYCARMELWSYYFDYWGQGTLVTVSSGGGGSGGGGSDV
VMTQSPSTLSASVGDRVTINCQASESISSWLAWYQQKPGKAPKLLIYEASKLASGVPSRFSGSGSGTEFTLTISSLQPDDFATYYC
QGYFYFISRTYVNSFGCGTKVEIKRTVAAPSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGECGGGGSGGGGSGGGGSQSALTQPASVSGSPGQSITISCTGTS
SDVGGYNFVSWYQQHPGKAPKLMIYDVSDRPSGVSDRFSGSKSGNTASLIISGLQADDEADYYCSSYGSSSTHVIFGGGTKVTVLG
GGGSGGGGSGGGGSGGGGSQVQLQESGGGLVKPGGSLSLSCAASGFTFSSYWMSWVRQAPGKGLEWVANINRDGSASYYVDSVKGR
FTISRDDAKNSLYLQMNSLRAEDTAVYYCARDRGVGYFDLWGRGTLVTVSS
Sequence ID 78: SI-55H11 Light Chain Nucleotide Sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAAGTGACCGTCCTAGGCGGTGGCGGTAGTGGGGGAGGCGG
TTCTGGCGGCGGAGGGTCCGGCGGTGGAGGATCACAGGTCACATTGAAGGAATCTGGCCCCGGCCTTGTTCAGCCAGGACAGACCC
TTAGCCTCACCTGTGCCTTCAGTGGTTTTTCTCTTAGCACTAGCGGTATGGGGGTCGGCTGGATTCGGCAGCCTCCCGGCAAAGGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCTCGGATGGAACTCTGGT
CTTACTACTTTGATTATTGGGGGCAGGGGACTCTCGTCACGGTCTCGAGTGGCGGTGGAGGGTCCGGCGGTGGTGGATCAGACGTC
GTGATGACCCAGTCTCCTTCCACCCTGTCTGCATCTGTAGGAGACAGAGTCACCATCAATTGCCAAGCCAGTGAGAGCATTAGCAG
TTGGTTAGCCTGGTATCAGCAGAAACCAGGGAAAGCCCCTAAGCTCCTGATCTATGAAGCATCCAAACTGGCATCTGGGGTCCCAT
CAAGGTTCAGCGGCAGTGGATCTGGGACAGAATTTACTCTCACCATCAGCAGCCTGCAGCCTGATGATTTTGCAACTTATTACTGC
CAAGGCTATTTTTATTTTATTAGTCGTACTTATGTAAATTCTTTCGGCTGTGGGACCAAGGTGGAGATCAAACGTACGGTGGCTGC
ACCATCTGTCTTCATCTTCCCGCCATCTGATGAGCAGTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGTAACTCCCAGGAGAGTGTCACAGAGCAGGACAGCAAG
GACAGCACCTACAGCCTCAGCAGCACCCTGACGCTGAGCAAAGCAGACTACGAGAAACACAAAGTCTACGCCTGCGAAGTCACCCA
TCAGGGCCTGAGCTCGCCCGTCACAAAGAGCTTCAACAGGGGAGAGTGTGGCGGTGGCGGTAGCGGTGGCGGCGGAAGTGGTGGCG
GAGGATCCCAGTCTGCCCTGACTCAGCCTGCCTCCGTGTCTGGGTCTCCTGGACAGTCGATCACCATCTCCTGCACTGGAACCAGC
AGTGACGTTGGTGGTTATAACTTTGTCTCCTGGTACCAACAACACCCAGGCAAAGCCCCCAAACTCATGATCTATGATGTCAGTGA
TCGGCCCTCAGGGGTGTCTGATCGCTTCTCCGGCTCCAAGTCTGGCAACACGGCCTCCCTGATCATCTCTGGCCTCCAGGCTGACG
ACGAGGCTGATTATTACTGCAGCTCATATGGGAGCAGCAGCACTCATGTGATTTTCGGCGGAGGGACCAAGGTGACCGTCCTAGGT
GGAGGCGGTTCAGGCGGAGGTGGTTCCGGCGGTGGCGGCTCCGGTGGAGGCGGCTCTCAGGTGCAATTGCAGGAGTCGGGGGGAGG
CCTGGTCAAGCCTGGAGGGTCCCTGAGTCTCTCCTGTGCAGCCTCTGGATTCACCTTTAGTAGTTATTGGATGAGCTGGGTCCGCC
AGGCTCCAGGGAAGGGGCTGGAGTGGGTGGCCAACATAAACCGCGATGGAAGTGCGAGTTACTATGTGGACTCTGTGAAGGGCCGA
TTCACCATCTCCAGAGACGACGCCAAGAACTCACTGTATCTGCAAATGAACAGCCTGAGAGCTGAGGACACGGCTGTGTATTACTG
TGCGAGAGATCGTGGGGTGGGCTACTTCGATCTCTGGGGCCGTGGCACCCTGGTCACCGTCTCTAGCTGA
>Sequence ID 79: SI-38E17 Heavy Chain Amino Acid Sequence
DVVMTQSPSTLSASVGDRVTINCQASESISSWLAWYQQKPGKAPKLLIYEASKLASGVPSRFSGSGSGTEFTLTISSLQPDDFATY
YCQGYFYFISRTYVNSFGGGTKVEIKGGGGSGGGGSGGGGSGGGGSEVQLVESGGGLVQPGGSLRLSCAASGFTISTNAMSWVRQA
PGKGLEWIGVITGRDITYYASWAKGRFTISRDNSKNTLYLQMNSLRAEDTAVYYCARDGGSSAITSNNIWGQGTLVTVSSGGGGSG
GGGSEVQLVQSGAEVKKPGESLKISCKGSGYSFSSSWIGWVRQAPGKGLEWMGIIYPDDSDTRYSPSFQGQVTISADKSIRTAYLQ
WSSLKASDTAMYYCARHVTMIWGVIIDFWGQGTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKRVEPKSCDKTHTCPPCPAPEAAGAPSVFLFPPKPKDT
LMISRTPEVTCVVVDVSHEDPEVKENWYVDGVEVHNAKTKPREEQYNSTYRVVSVLTVLHQDWLNGKEYKCAVSNKALPAPIEKTI
SKAKGQPREPQVYTLPPSRDELTKNQVSLTCLVKGFYPSDIAVEWESNGQPENNYKTTPPVLDSDGSFFLYSKLTVDKSRWQQGNV
FSCSVMHEALHNHYTQKSLSLSPGGGGGSGGGGSEVQLLESGGGLVQPGGSLRLSCAASGFSFSSGYDMCWVRQAPGKGLEWIACI
AAGSAGITYDANWAKGRFTISRDNSKNTLYLQMNSLRAEDTAVYYCARSAFSFDYAMDLWGQGTLVTVSSGGGGSGGGGSGGGGSG
GGGSDIQMTQSPSTLSASVGDRVTITCQASQSISSHLNWYQQKPGKAPKLLIYKASTLASGVPSRFSGSGSGTEFTLTISSLQPDD
FATYYCQQGYSWGNVDNVFGGGTKVEIKGGGGSGGGGSRSLVESGGGLVQPGGSLRLSCTASGFTISSYHMQWVRQAPGKGLEYIG
TISSGGNVYYASSARGRFTISRPSSKNTVDLQMNSLRAEDTAVYYCARDSGYSDPMWGQGTLVTVSSGGGGSGGGGSGGGGSGGGG
SDVVMTQSPSSVSASVGDRVTITCQASQNIRTYLSWYQQKPGKAPKLLIYAAANLASGVPSRFSGSGSGTDFTLTISDLEPGDAAT
YYCQSTYLGTDYVGGAFGGGTKVEIK
Sequence ID 80: SI-38E17 Heavy Chain Amino Acid Sequence
GACGTCGTGATGACCCAGTCTCCTTCCACCCTGTCTGCATCTGTAGGAGACAGAGTCACCATCAATTGCCAAGCCAGTGAGAGCAT
TAGCAGTTGGTTAGCCTGGTATCAGCAGAAACCAGGGAAAGCCCCTAAGCTCCTGATCTATGAAGCATCCAAACTGGCATCTGGGG
TCCCATCAAGGTTCAGCGGCAGTGGATCTGGGACAGAGTTCACTCTCACCATCAGCAGCCTGCAGCCTGATGATTTTGCAACTTAT
TACTGCCAAGGCTATTTTTATTTTATTAGTCGTACTTATGTAAATTCTTTCGGCGGAGGGACCAAGGTGGAGATCAAAGGCGGTGG
CGGTAGTGGGGGAGGCGGTTCTGGCGGCGGAGGGTCCGGCGGTGGAGGATCAGAGGTGCAGCTGGTGGAGTCTGGGGGAGGCTTGG
TCCAGCCTGGGGGGTCCCTGAGACTCTCCTGTGCAGCCTCTGGATTCACCATCAGTACCAATGCAATGAGCTGGGTCCGCCAGGCT
CCAGGGAAGGGGCTGGAGTGGATCGGAGTCATTACTGGTCGTGATATCACATACTACGCGAGCTGGGCGAAAGGCAGATTCACCAT
CTCCAGAGACAATTCCAAGAACACGCTGTATCTTCAAATGAACAGCCTGAGAGCCGAGGACACGGCTGTGTATTACTGTGCGCGCG
ACGGTGGATCATCTGCTATTACTAGTAACAACATTTGGGGCCAAGGAACTCTGGTCACCGTTTCTTCAGGCGGTGGAGGGTCCGGC
GGTGGTGGATCCGAGGTGCAGCTGGTGCAGTCTGGAGCAGAGGTGAAGAAACCAGGAGAGTCTCTGAAGATCTCCTGTAAGGGTTC
TGGATACAGCTTTAGCAGTTCATGGATCGGCTGGGTGCGCCAGGCACCTGGGAAAGGCCTGGAATGGATGGGGATCATCTATCCTG
ATGACTCTGATACCAGATACAGTCCATCCTTCCAAGGCCAGGTCACCATCTCAGCCGACAAGTCCATCAGGACTGCCTACCTGCAG
TGGAGTAGCCTGAAGGCCTCGGACACCGCTATGTATTACTGTGCGAGACATGTTACTATGATTTGGGGAGTTATTATTGACTTCTG
GGGCCAGGGAACCCTGGTCACCGTCTCCTCAGCTAGCACCAAGGGCCCATCGGTCTTCCCCCTGGCACCCTCCTCCAAGAGCACCT
CTGGGGGCACAGCGGCCCTGGGCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTGACCGTGCCCTCCAGCAGCTT
GGGCACCCAGACCTACATCTGCAACGTGAATCACAAGCCCAGCAACACCAAGGTGGACAAGAGAGTTGAGCCCAAATCTTGTGACA
AAACTCACACATGCCCACCGTGCCCAGCACCTGAAGCCGCGGGGGCACCGTCAGTCTTCCTCTTCCCCCCAAAACCCAAGGACACC
CTCATGATCTCCCGGACCCCTGAGGTCACATGCGTGGTGGTGGACGTGAGCCACGAAGACCCTGAGGTCAAGTTCAACTGGTACGT
GGACGGCGTGGAGGTGCATAATGCCAAGACAAAGCCGCGGGAGGAGCAGTACAACAGCACGTACCGTGTGGTCAGCGTCCTCACCG
TCCTGCACCAGGACTGGCTGAATGGCAAGGAGTACAAGTGCGCGGTCTCCAACAAAGCCCTCCCAGCCCCCATCGAGAAAACCATC
TCCAAAGCCAAAGGGCAGCCCCGAGAACCACAGGTGTATACCCTGCCCCCATCCCGGGATGAGCTGACCAAGAACCAGGTCAGCCT
GACCTGCCTGGTCAAAGGCTTCTATCCCAGCGACATCGCCGTGGAGTGGGAGAGCAATGGGCAGCCGGAGAACAACTACAAGACCA
CGCCTCCCGTGCTGGACTCCGACGGCTCCTTCTTCCTCTATAGCAAGCTCACCGTGGACAAGAGCAGGTGGCAGCAGGGGAACGTC
TTCTCATGCTCCGTGATGCATGAGGCTCTGCACAACCACTACACGCAGAAGAGCCTCTCCCTGTCTCCGGGTGGCGGTGGAGGGTC
CGGCGGTGGTGGATCCGAGGTGCAGCTGTTGGAGTCTGGGGGAGGCTTGGTACAGCCTGGGGGGTCCCTGAGACTCTCCTGTGCAG
CCTCTGGATTCTCCTTCAGTAGCGGGTACGACATGTGCTGGGTCCGCCAGGCTCCAGGGAAGGGGCTGGAGTGGATCGCATGCATT
GCTGCTGGTAGTGCTGGTATCACTTACGACGCGAACTGGGCGAAAGGCCGGTTCACCATCTCCAGAGACAATTCCAAGAACACGCT
GTATCTGCAAATGAACAGCCTGAGAGCCGAGGACACGGCCGTATATTACTGTGCGAGATCGGCGTTTTCGTTCGACTACGCCATGG
ACCTCTGGGGCCAGGGAACCCTGGTCACCGTCTCGAGCGGTGGAGGCGGATCTGGCGGAGGTGGTTCCGGCGGTGGCGGCTCCGGT
GGAGGCGGCTCTGACATCCAGATGACCCAGTCTCCTTCCACCCTGTCTGCATCTGTAGGAGACAGAGTCACCATCACTTGCCAGGC
CAGTCAGAGCATTAGTTCCCACTTAAACTGGTATCAGCAGAAACCAGGGAAAGCCCCTAAGCTCCTGATCTATAAGGCATCCACTC
TGGCATCTGGGGTCCCATCAAGGTTCAGCGGCAGTGGATCTGGGACAGAATTTACTCTCACCATCAGCAGCCTGCAGCCTGATGAT
TTTGCAACTTATTACTGCCAACAGGGTTATAGTTGGGGTAATGTTGATAATGTTTTCGGCGGAGGGACCAAGGTGGAGATCAAAGG
CGGTGGAGGGTCCGGCGGTGGTGGATCCCGGTCGCTGGTGGAGTCTGGGGGAGGCTTGGTCCAGCCTGGGGGGTCCCTGAGACTCT
CCTGTACAGCCTCTGGATTCACCATCAGTAGCTACCACATGCAGTGGGTCCGCCAGGCTCCAGGGAAGGGGCTGGAGTACATCGGA
ACCATTAGTAGTGGTGGTAATGTATACTACGCGAGCTCCGCGAGAGGCAGATTCACCATCTCCAGACCCTCGTCCAAGAACACGGT
GGATCTTCAAATGAACAGCCTGAGAGCCGAGGACACGGCTGTGTATTACTGTGCGAGAGACTCTGGTTATAGTGATCCTATGTGGG
GCCAGGGAACCCTGGTCACCGTCTCGAGCGGCGGTGGCGGTAGTGGGGGAGGCGGTTCTGGCGGCGGAGGGTCCGGCGGTGGAGGA
TCAGACGTTGTGATGACCCAGTCTCCATCTTCCGTGTCTGCATCTGTAGGAGACAGAGTCACCATCACCTGTCAGGCCAGTCAGAA
CATTAGGACTTACTTATCCTGGTATCAGCAGAAACCAGGGAAAGCCCCTAAGCTCCTGATCTATGCTGCAGCCAATCTGGCATCTG
GGGTCCCATCAAGGTTCAGCGGCAGTGGATCTGGGACAGATTTCACTCTCACCATCAGCGACCTGGAGCCTGGCGATGCTGCAACT
TACTATTGTCAGTCTACCTATCTTGGTACTGATTATGTTGGCGGTGCTTTCGGCGGAGGGACCAAGGTGGAGATCAAA
Sequence ID 81: SI-38E17 Light Chain Amino Acid Sequence
AIQLTQSPSSLSASVGDRVTITCRASQGISSALAWYQQKPGKAPKLLIYDASSLESGVPSRFSGSGSGTDFTLTISSLQPEDFATY
YCQQFNSYPFTFGPGTKVDIKRTVAAPSVFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC
>Sequence ID 82: SI-38E17 Light Chain Nucleotide Sequence
GCCATCCAGTTGACCCAGTCTCCATCCTCCCTGTCTGCATCTGTAGGAGACAGAGTCACCATCACTTGCCGGGCAAGTCAGGGCAT
TAGCAGTGCTTTAGCCTGGTATCAGCAGAAACCAGGGAAAGCTCCTAAGCTCCTGATCTATGATGCCTCCAGTTTGGAAAGTGGGG
TCCCATCAAGGTTCAGCGGCAGTGGATCTGGGACAGATTTCACTCTCACCATCAGCAGCCTGCAGCCTGAAGATTTTGCAACTTAT
TACTGTCAACAGTTTAATAGTTACCCATTCACTTTCGGCCCTGGGACCAAAGTGGATATCAAACGTACGGTGGCTGCACCATCTGT
CTTCATCTTCCCGCCATCTGATGAGCAGTTGAAATCTGGAACTGCCTCTGTTGTGTGCCTGCTGAATAACTTCTATCCCAGAGAGG
CCAAAGTACAGTGGAAGGTGGATAACGCCCTCCAATCGGGTAACTCCCAGGAGAGTGTCACAGAGCAGGACAGCAAGGACAGCACC
TACAGCCTCAGCAGCACCCTGACGCTGAGCAAAGCAGACTACGAGAAACACAAAGTCTACGCCTGCGAAGTCACCCATCAGGGCCT
GAGCTCGCCCGTCACAAAGAGCTTCAACAGGGGAGAGTGT
Sequence ID 83: SI-34C1 Heavy Chain Amino Acid Sequence
EVQLVQSGAEVKKPGESLKISCKGSGYSFSSSWIGWVRQAPGKGLEWMGIIYPDDSDTRYSPSFQGQVTISADKSIRTAYLQWSSL
KASDTAMYYCARHVTMIWGVIIDFWGQGTLVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTCPPCPAPELLGGPSVFLFPPKPKDTLMIS
RTPEVTCVVVDVSHEDPEVKFNWYVDGVEVHNAKTKPREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENNYKTTPPVLDSDGSFFLYSKLTVDKSRWQQGNVFSCS
VMHEALHNHYTQKSLSLSPG
Sequence ID 84: SI-34C1 Heavy Chain Nucleotide Sequence
GAGGTGCAGCTGGTGCAGTCTGGAGCAGAGGTGAAGAAACCAGGAGAGTCTCTGAAGATCTCCTGTAAGGGTTCTGGATACAGCTT
TAGCAGTTCATGGATCGGCTGGGTGCGCCAGGCACCTGGGAAAGGCCTGGAATGGATGGGGATCATCTATCCTGATGACTCTGATA
CCAGATACAGTCCATCCTTCCAAGGCCAGGTCACCATCTCAGCCGACAAGTCCATCAGGACTGCCTACCTGCAGTGGAGTAGCCTG
AAGGCCTCGGACACCGCTATGTATTACTGTGCGAGACATGTTACTATGATTTGGGGAGTTATTATTGACTTCTGGGGCCAGGGAAC
CCTGGTCACCGTCTCCTCAGCTAGCACCAAGGGCCCATCGGTCTTCCCCCTGGCACCCTCCTCCAAGAGCACCTCTGGGGGCACAG
CGGCCCTGGGCTGCCTGGTCAAGGACTACTTCCCCGAACCGGTGACGGTGTCGTGGAACTCAGGCGCCCTGACCAGCGGCGTGCAC
ACCTTCCCGGCTGTCCTACAGTCCTCAGGACTCTACTCCCTCAGCAGCGTGGTGACCGTGCCCTCCAGCAGCTTGGGCACCCAGAC
CTACATCTGCAACGTGAATCACAAGCCCAGCAACACCAAGGTGGACAAGAGAGTTGAGCCCAAATCTTGTGACAAAACTCACACAT
GCCCACCGTGCCCAGCACCTGAACTCCTGGGGGGACCGTCAGTCTTCCTCTTCCCCCCAAAACCCAAGGACACCCTCATGATCTCC
CGGACCCCTGAGGTCACATGCGTGGTGGTGGACGTGAGCCACGAAGACCCTGAGGTCAAGTTCAACTGGTACGTGGACGGCGTGGA
GGTGCATAATGCCAAGACAAAGCCGCGGGAGGAGCAGTACAACAGCACGTACCGTGTGGTCAGCGTCCTCACCGTCCTGCACCAGG
ACTGGCTGAATGGCAAGGAGTACAAGTGCAAGGTCTCCAACAAAGCCCTCCCAGCCCCCATCGAGAAAACCATCTCCAAAGCCAAA
GGGCAGCCCCGAGAACCACAGGTGTACACCCTGCCCCCATCCCGGGATGAGCTGACCAAGAACCAGGTCAGCCTGACCTGCCTGGT
CAAAGGCTTCTATCCCAGCGACATCGCCGTGGAGTGGGAGAGCAATGGGCAGCCGGAGAACAACTACAAGACCACGCCTCCCGTGC
TGGACTCCGACGGCTCCTTCTTCCTCTATAGCAAGCTCACCGTGGACAAGAGCAGGTGGCAGCAGGGGAACGTCTTCTCATGCTCC
GTGATGCATGAGGCTCTGCACAACCACTACACGCAGAAGAGCCTCTCCCTGTCTCCGGGT
>Sequence ID 85: SI-34C1 Light Chain Amino Acid Sequence
AIQLTQSPSSLSASVGDRVTITCRASQGISSALAWYQQKPGKAPKLLIYDASSLESGVPSRFSGSGSGTDFTLTISSLQPEDFATY
YCQQFNSYPFTFGPGTKVDIKRTVAAPSVFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC
>Sequence ID 86: SI-34C1 Light Chain Nucleotide Sequence
GCCATCCAGTTGACCCAGTCTCCATCCTCCCTGTCTGCATCTGTAGGAGACAGAGTCACCATCACTTGCCGGGCAAGTCAGGGCAT
TAGCAGTGCTTTAGCCTGGTATCAGCAGAAACCAGGGAAAGCTCCTAAGCTCCTGATCTATGATGCCTCCAGTTTGGAAAGTGGGG
TCCCATCAAGGTTCAGCGGCAGTGGATCTGGGACAGATTTCACTCTCACCATCAGCAGCCTGCAGCCTGAAGATTTTGCAACTTAT
TACTGTCAACAGTTTAATAGTTACCCATTCACTTTCGGCCCTGGGACCAAAGTGGATATCAAACGTACGGTGGCTGCACCATCTGT
CTTCATCTTCCCGCCATCTGATGAGCAGTTGAAATCTGGAACTGCCTCTGTTGTGTGCCTGCTGAATAACTTCTATCCCAGAGAGG
CCAAAGTACAGTGGAAGGTGGATAACGCCCTCCAATCGGGTAACTCCCAGGAGAGTGTCACAGAGCAGGACAGCAAGGACAGCACC
TACAGCCTCAGCAGCACCCTGACGCTGAGCAAAGCAGACTACGAGAAACACAAAGTCTACGCCTGCGAAGTCACCCATCAGGGCCT
GAGCTCGCCCGTCACAAAGAGCTTCAACAGGGGAGAGTGT
>Sequence ID 87: SI-38P12 Heavy Chain Amino Acid Sequence
QIVLSQSPAILSASPGEKVTMTCRASSSVSYIHWFQQKPGSSPKPWIYATSNLASGVPVRFSGSGSGTSYSLTISRVEAEDAATYY
CQQWTSNPPTFGGGTKLTVLGGGGSGGGGSGGGGSGGGGSQVQLQQPGAELVKPGASVKMSCKASGYTFTSYNMHWVKQTPGRGLE
WIGAIYPGNGDTSYNQKFKGKATLTADKSSSTAYMQLSSLTSEDSAVYYCARSTYYGGDWYFNVWGAGTTVTVSSGGGGSGGGGSE
VQLVESGGGLVQPGGSLRLSCAASGFTISTNAMSWVRQAPGKGLEWIGVITGRDITYYASWAKGRFTISRDNSKNTLYLQMNSLRA
EDTAVYYCARDGGSSAITSNNIWGQGTLVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDKTHTCPPCPAPEAAGAPSVFLFPPKPKDTLMISRT
PEVTCVVVDVSHEDPEVKFNWYVDGVEVHNAKTKPREEQYNSTYRVVSVLTVLHQDWLNGKEYKCAVSNKALPAPIEKTISKAKGQ
PREPQVYTLPPSRDELTKNQVSLTCLVKGFYPSDIAVEWESNGQPENNYKTTPPVLDSDGSFFLYSKLTVDKSRWQQGNVFSCSVM
HEALHNHYTQKSLSLSPGGGGGSGGGGSEVQLLESGGGLVQPGGSLRLSCAASGFSFSSGYDMCWVRQAPGKGLEWIACIAAGSAG
ITYDANWAKGRFTISRDNSKNTLYLQMNSLRAEDTAVYYCARSAFSFDYAMDLWGQGTLVTVSSGGGGSGGGGSGGGGSGGGGSDI
QMTQSPSTLSASVGDRVTITCQASQSISSHLNWYQQKPGKAPKLLIYKASTLASGVPSRFSGSGSGTEFTLTISSLQPDDFATYYC
QQGYSWGNVDNVFGGGTKVEIKGGGGSGGGGSGRSLVESGGGLVQPGGSLRLSCTASGFTISSYHMQWVRQAPGKGLEYIGTISSG
GNVYYASSARGRFTISRPSSKNTVDLOMNSLRAEDTAVYYCARDSGYSDPMWGOGTLVTVSSGGGGSGGGGSGGGGSGGGGSDVVM
TQSPSSVSASVGDRVTITCQASQNIRTYLSWYQQKPGKAPKLLIYAAANLASGVPSRFSGSGSGTDETLTISDLEPGDAATYYCQS
TYLGTDYVGGAFGGGTKVEIK
Sequence ID 88: SI-38P12 Heavy Chain Nucleotide Sequence
CAGATCGTGCTGAGCCAGAGCCCCGCCATCCTGAGCGCCAGCCCCGGCGAGAAGGTGACCATGACCTGCCGGGCCAGCAGCAGCGT
GAGCTACATCCACTGGTTCCAGCAGAAGCCCGGCAGCAGCCCCAAGCCCTGGATCTACGCCACCAGCAACCTGGCCAGCGGCGTGC
CCGTGCGGTTCAGCGGCAGCGGCAGCGGCACCAGCTACAGCCTGACCATCAGCCGGGTGGAGGCCGAGGACGCCGCCACCTACTAC
TGCCAGCAGTGGACCAGCAACCCCCCCACCTTCGGCGGCGGCACCAAGCTGACCGTGCTGGGTGGTGGTGGCTCTGGAGGAGGCGG
GAGCGGGGGTGGTGGCTCAGGTGGTGGAGGTTCCCAGGTGCAGCTGCAGCAGCCCGGCGCCGAGCTGGTGAAGCCCGGCGCCAGCG
TGAAGATGAGCTGCAAGGCCAGCGGCTACACCTTCACCAGCTACAACATGCACTGGGTGAAGCAGACCCCCGGCCGGGGCCTGGAG
TGGATCGGCGCCATCTACCCCGGCAACGGCGACACCAGCTACAACCAGAAGTTCAAGGGCAAGGCCACCCTGACCGCCGACAAGAG
CAGCAGCACCGCCTACATGCAGCTGAGCAGCCTGACCAGCGAGGACAGCGCCGTGTACTACTGCGCCCGGAGCACCTACTACGGCG
GCGACTGGTACTTCAACGTGTGGGGCGCCGGCACCACCGTGACCGTCTCGAGTGGCGGTGGAGGGTCCGGCGGTGGTGGATCAGAG
GTGCAGCTGGTGGAGTCTGGGGGAGGCTTGGTCCAGCCTGGGGGGTCCCTGAGACTCTCCTGTGCAGCCTCTGGATTCACCATCAG
TACCAATGCAATGAGCTGGGTCCGCCAGGCTCCAGGGAAGGGGCTGGAGTGGATCGGAGTCATTACTGGTCGTGATATCACATACT
ACGCGAGCTGGGCGAAAGGCAGATTCACCATCTCCAGAGACAATTCCAAGAACACGCTGTATCTTCAAATGAACAGCCTGAGAGCC
GAGGACACGGCTGTGTATTACTGTGCGAGAGACGGTGGTTCTTCTGCTATTACTAGTAACAACATTTGGGGCCAGGGAACCCTGGT
CACCGTGTCCTCAGCTAGCACCAAGGGCCCATCGGTCTTCCCCCTGGCACCCTCCTCCAAGAGCACCTCTGGGGGCACAGCGGCCC
TGGGCTGCCTGGTCAAGGACTACTTCCCCGAACCGGTGACGGTGTCGTGGAACTCAGGCGCCCTGACCAGCGGCGTGCACACCTTC
CCGGCTGTCCTACAGTCCTCAGGACTCTACTCCCTCAGCAGCGTGGTGACCGTGCCCTCCAGCAGCTTGGGCACCCAGACCTACAT
CTGCAACGTGAATCACAAGCCCAGCAACACCAAGGTGGACAAGAGAGTTGAGCCCAAATCTTGTGACAAAACTCACACATGCCCAC
CGTGCCCAGCACCTGAAGCCGCGGGGGCACCGTCAGTCTTCCTCTTCCCCCCAAAACCCAAGGACACCCTCATGATCTCCCGGACC
CCTGAGGTCACATGCGTGGTGGTGGACGTGAGCCACGAAGACCCTGAGGTCAAGTTCAACTGGTACGTGGACGGCGTGGAGGTGCA
TAATGCCAAGACAAAGCCGCGGGAGGAGCAGTACAACAGCACGTACCGTGTGGTCAGCGTCCTCACCGTCCTGCACCAGGACTGGC
TGAATGGCAAGGAGTACAAGTGCGCGGTCTCCAACAAAGCCCTCCCAGCCCCCATCGAGAAAACCATCTCCAAAGCCAAAGGGCAG
CCCCGAGAACCACAGGTGTACACCCTGCCCCCATCCCGGGATGAGCTGACCAAGAACCAGGTCAGCCTGACCTGCCTGGTCAAAGG
CTTCTATCCCAGCGACATCGCCGTGGAGTGGGAGAGCAATGGGCAGCCGGAGAACAACTACAAGACCACGCCTCCCGTGCTGGACT
CCGACGGCTCCTTCTTCCTCTATAGCAAGCTCACCGTGGACAAGAGCAGGTGGCAGCAGGGGAACGTCTTCTCATGCTCCGTGATG
CATGAGGCTCTGCACAACCACTACACGCAGAAGAGCCTCTCCCTGTCTCCGGGTGGCGGTGGAGGGTCCGGCGGTGGTGGATCCGA
GGTGCAGCTGTTGGAGTCTGGGGGAGGCTTGGTACAGCCTGGGGGGTCCCTGAGACTCTCCTGTGCAGCCTCTGGATTCTCCTTCA
GTAGCGGGTACGACATGTGCTGGGTCCGCCAGGCTCCAGGGAAGGGGCTGGAGTGGATCGCATGCATTGCTGCTGGTAGTGCTGGT
ATCACTTACGACGCGAACTGGGCGAAAGGCCGGTTCACCATCTCCAGAGACAATTCCAAGAACACGCTGTATCTGCAAATGAACAG
CCTGAGAGCCGAGGACACGGCCGTATATTACTGTGCGAGATCGGCGTTTTCGTTCGACTACGCCATGGACCTCTGGGGCCAGGGAA
CCCTGGTCACCGTGTCGAGCGGTGGAGGCGGATCTGGCGGAGGTGGTTCCGGCGGTGGCGGCTCCGGTGGAGGCGGCTCTGACATC
CAGATGACCCAGTCTCCTTCCACCCTGTCTGCATCTGTAGGAGACAGAGTCACCATCACTTGCCAGGCCAGTCAGAGCATTAGTTC
CCACTTAAACTGGTATCAGCAGAAACCAGGGAAAGCCCCTAAGCTCCTGATCTATAAGGCATCCACTCTGGCATCTGGGGTCCCAT
CAAGGTTCAGCGGCAGTGGATCTGGGACAGAATTTACTCTCACCATCAGCAGCCTGCAGCCTGATGATTTTGCAACTTATTACTGC
CAACAGGGTTATAGTTGGGGTAATGTTGATAATGTTTTCGGCGGAGGGACCAAGGTGGAGATCAAAGGCGGTGGAGGGTCCGGCGG
TGGTGGCTCCGGACGGTCGCTGGTGGAGTCTGGGGGAGGCTTGGTCCAGCCTGGGGGGTCCCTGAGACTCTCCTGTACTGCCTCTG
GATTCACCATCAGTAGCTACCACATGCAGTGGGTCCGCCAGGCTCCAGGGAAGGGGCTGGAGTACATCGGAACCATTAGTAGTGGT
GGTAATGTATACTACGCAAGCTCCGCTAGAGGCAGATTCACCATCTCCAGACCCTCGTCCAAGAACACGGTGGATCTTCAAATGAA
CAGCCTGAGAGCCGAGGACACGGCTGTGTATTACTGTGCGAGAGACTCTGGTTATAGTGATCCTATGTGGGGCCAGGGAACCCTGG
TCACCGTCTCTTCAGGCGGTGGCGGTAGTGGGGGAGGCGGTTCTGGCGGCGGAGGGTCCGGCGGTGGAGGATCAGACGTTGTGATG
ACCCAGTCTCCATCTTCCGTGTCTGCATCTGTAGGAGACAGAGTCACCATCACCTGTCAGGCCAGTCAGAACATTAGGACTTACTT
ATCCTGGTATCAGCAGAAACCAGGGAAAGCCCCTAAGCTCCTGATCTATGCTGCAGCCAATCTGGCATCTGGGGTCCCATCAAGGT
TCAGCGGCAGTGGATCTGGGACAGATTTCACTCTCACCATCAGCGACCTGGAGCCTGGCGATGCTGCAACTTACTATTGTCAGTCT
ACCTATCTTGGTACTGATTATGTTGGCGGTGCTTTCGGCGGAGGGACCAAGGTGGAGATCAAATGA
>Sequence ID 89: SI-38P12 Light Chain Amino Acid Sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKVTVLGGGGSGGGGSGGGGSGGGGSQVTLKESGPGLVQPGQTLRLTCAFSGFSLSTSGMGVGWIRQPPGKG
LEWLAHIWWDDDKRYNPALKSRLTISKDTSKNQVYLQMNSLDAEDTAVYYCARMELWSYYFDYWGQGTLVTVSSGGGGSGGGGSDV
VMTQSPSTLSASVGDRVTINCQASESISSWLAWYQQKPGKAPKLLIYEASKLASGVPSRFSGSGSGTEFTLTISSLQPDDFATYYC
QGYFYFISRTYVNSFGGGTKVEIKRTVAAPSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGEC
>Sequence ID 90: SI-38P12 Light Chain Nucleotide Sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAAGTGACGGTACTGGGTGGAGGCGGTTCAGGCGGAGGTGG
TTCCGGCGGTGGCGGCTCCGGTGGAGGCGGCTCTCAGGTCACATTGAAGGAATCTGGCCCCGGCCTTGTTCAGCCAGGACAGACCC
TTAGGCTCACCTGTGCCTTCAGTGGTTTTTCTCTTAGCACTAGCGGTATGGGGGTCGGCTGGATTCGGCAGCCTCCCGGCAAAGGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCGCGGATGGAACTCTGGT
CTTACTACTTTGATTATTGGGGGCAGGGGACTCTCGTCACGGTCTCGAGCGGCGGTGGAGGGTCCGGCGGTGGTGGATCAGACGTC
GTGATGACCCAGTCTCCTTCCACCCTGTCTGCATCTGTAGGAGACAGAGTCACCATCAATTGCCAAGCCAGTGAGAGCATTAGCAG
TTGGTTAGCCTGGTATCAGCAGAAACCAGGGAAAGCCCCTAAGCTCCTGATCTATGAAGCATCCAAACTGGCATCTGGGGTCCCAT
CAAGGTTCAGCGGCAGTGGATCTGGGACAGAATTCACTCTCACCATCAGCAGCCTGCAGCCTGATGATTTTGCAACTTATTACTGC
CAAGGCTATTTTTATTTTATTAGTCGTACTTATGTAAATTCTTTCGGCGGAGGGACCAAGGTGGAGATCAAACGTACGGTGGCTGC
ACCATCTGTCTTCATCTTCCCGCCATCTGATGAGCAGTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGTAACTCCCAGGAGAGTGTCACAGAGCAGGACAGCAAG
GACAGCACCTACAGCCTCAGCAGCACCCTGACGCTGAGCAAAGCAGACTACGAGAAACACAAAGTCTACGCCTGCGAAGTCACCCA
TCAGGGCCTGAGCTCGCCCGTCACAAAGAGCTTCAACAGGGGAGAGTGT
Sequence ID 91: Humanized Version 7 (H7VH) Amino Acid Sequence
QVTLKESGPGLVQPGQTLRLTCAFSGFSLSTSGMGVGWIRQPPGKCLEWLAHIWWDDDKRYNPALKSRLTISKDTSKNQVYLQMNS
LDAEDTAVYYCARMELWSYYFDYWGQGTLVTVSS
>Sequence ID 92: Humanized Version 7 (H7VH) Nucleotide Sequence
CAGGTCACATTGAAGGAATCTGGCCCCGGCCTTGTTCAGCCAGGACAGACCCTTAGGCTCACCTGTGCCTTCAGTGGTTTTTCTCT
TAGCACTAGCGGTATGGGGGTCGGCTGGATTCGGCAGCCTCCCGGCAAATGTCTTGAGTGGTTGGCTCACATTTGGTGGGACGACG
ACAAACGGTATAATCCTGCCTTGAAAAGTCGGCTGACCATTAGTAAGGATACCTCAAAAAATCAAGTGTACTTGCAAATGAATAGC
CTTGACGCCGAGGATACGGCTGTATATTATTGCGCTCGGATGGAACTCTGGTCTTACTACTTTGATTATTGGGGGCAGGGGACTCT
CGTCACGGTGTCGAGT
>Sequence ID 93: Humanized Version 7 (H7VL) Amino Acid Sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGCGTKVEIK
>Sequence ID 94: Humanized Version 7 (H7VL) Nucleotide Sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGTGTGGGACAAAAGTGGAGATCAAG
>Sequence ID 95: SI-63SV7 Amino Acid Sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGCGTKVEIKGGGGSGGGGSGGGGSGGGGSQVTLKESGPGLVQPGQTLRLTCAFSGFSLSTSGMGVGWIRQPPGKC
LEWLAHIWWDDDKRYNPALKSRLTISKDTSKNQVYLQMNSLDAEDTAVYYCARMELWSYYFDYWGQGTLVTVSS
>Sequence ID 96: SI-63SV7 Nucleic Acid Sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGTGTGGGACAAAAGTGGAGATCAAGGGTGGCGGAGGCAGTGGTGGCGGGGG
CAGCGGAGGTGGTGGTTCAGGGGGTGGTGGGAGCCAGGTCACATTGAAGGAATCTGGCCCCGGCCTTGTTCAGCCAGGACAGACCC
TTAGGCTCACCTGTGCCTTCAGTGGTTTTTCTCTTAGCACTAGCGGTATGGGGGTCGGCTGGATTCGGCAGCCTCCCGGCAAATGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCTCGGATGGAACTCTGGT
CTTACTACTTTGATTATTGGGGGCAGGGGACTCTCGTCACGGTGTCGAGT

What is claimed is:

1. A peptide having a binding specificity to human CD19, comprising an amino acid sequence having a sequence identity to SEQ ID NO. 1, 3, 5, 7, 9, 11, 13, 15, 17, 19, 21, 23, 91, or 93.

2-7. (canceled)

8. A multi-specific antibody-like protein, wherein the multi-specific antibody-like protein has a N-terminal and a C-terminal, comprising in tandem from the N-terminal to the C-terminal, a first binding domain (D1) at the N-terminal,
a second binding domain (D2) comprising a light chain moiety,
a Fc region,
a third binding domain (D3), and
a fourth binding domain (D4) at the C-terminal,
wherein the light chain moiety comprises a fifth binding domain (D5) covalently attached to the C-terminal, a sixth binding domain (D6) covalently attached to the N-terminal, or both,
wherein the D1, D2, D3, D4, D5 and D6 each has a binding specificity to a tumor antigen, an immune signaling antigen, or a combination thereof, and
wherein the multi-specific antibody-like protein comprises an amino acid sequence having a sequence identity to SEQ ID NO. 1, 3, 5, 7, 9, 11, 13, 15, 17, 19, 21, 23, 91, or 93.

9. (canceled)

10. The multi-specific antibody-like protein of claim 8, wherein D1 comprises a peptide having an amino acid sequence having a sequence identity to SEQ ID NO. 7 or 19.

11. (canceled)

12. The multi-specific antibody-like protein of claim 8, wherein D2 comprises a peptide having an amino acid sequence having a sequence identity to SEQ ID NO. 91 or 93.

13. (canceled)

14. The multi-specific antibody-like protein of claim 8, wherein D6 comprises a peptide having an amino acid sequence having a sequence identity to SEQ ID NO. 7 or 19.

15. The multi-specific antibody-like protein of claim 8, wherein the multi-specific antibody-like protein is a monoclonal antibody.

16. The multi-specific antibody-like protein of claim 15, having a binding affinity to human CD19 with a Kd not greater than 10 nM.

17. The multi-specific antibody-like protein of claim 15, comprising a scFv domain, a Fab region, or both, wherein the scFv domain or Fab region comprises an amino acid sequence having a sequence identity to SEQ ID NO. 1, 3, 5, 7, 9, 11, 13, 15, 17, 19, 21, 23, 91, or 93.

18. The multi-specific antibody-like protein of claim 15, wherein the antibody is an IgG, or wherein the antibody is a humanized antibody.

19. An isolated nucleic acid sequence, encoding an amino acid sequence of the multi-specific antibody-like protein of claim 15.

20. An expression vector comprising the isolated nucleic acid of claim 19.

21. A host cell comprising the nucleic acid of claim 19, wherein the host cell is a prokaryotic cell or a eukaryotic cell.

22. A method of producing an antibody comprising culturing the host cell of claim 21 so that the antibody is produced.

23. An immune-conjugate, comprising the multi-specific antibody-like protein of claim 15 and a drug unit, wherein the drug unit is linked to the multi-specific antibody-like protein through a linker, and wherein the linker comprises a covalent bond selected from an ester bond, an ether bond, an amine bond, an amide bond, a disulfide bond, an imide bond, a sulfone bond, a phosphate bond, a phosphorus ester bond, a peptide bond, a hydrazone bond or a combination thereof.

24. The immune-conjugate of claim 23, wherein the drug unit comprises a cytotoxic agent, an immune regulatory reagent, an imaging agent or a combination thereof.

25. The immune-conjugate of claim 24, wherein the cytotoxic agent is selected from a growth inhibitory agent or a chemotherapeutic agent from a class of tubulin binders, DNA intercalators, DNA alkylators, enzyme inhibitors, immune modulators, antimetabolite agents, radioactive isotopes, or a combination thereof, wherein the cytotoxic agent is selected from a calicheamicin, camptothecin, ozogamicin, monomethyl auristatin E, emtansine, a derivative or a combination thereof, or wherein the immune regulatory reagents activate or suppress immune cells, T cell, NK cell, B cell, macrophage, or dendritic cell.

26-28. (canceled)

29. A pharmaceutical composition, comprising the multi-specific antibody-like protein of claim 15 and a pharmaceutically acceptable carrier.

30. The pharmaceutical composition of claim 29, further comprising a chemotherapeutic agent, a growth inhibitory agent, a cytotoxic agent from class of calicheamicin, an antimitotic agent, a toxin, a radioactive isotope, a therapeutic agent, or a combination thereof.

31. A pharmaceutical composition, comprising the immune-conjugate of claim 24 and a pharmaceutically acceptable carrier.

32. A method of treating a subject with a cancer, comprising administering to the subject an effective amount of the multi-specific antibody-like protein of claim 15.

33. The method of claim 32, further comprising co-administering an effective amount of a therapeutic agent, wherein the therapeutic agent comprises an antibody, a chemotherapy agent, an enzyme, or a combination thereof.

34. The method of claim 33, wherein the subject is a human.

35. (canceled)


SEQUENCE LISTING (96 sequences)

SEQ ID NO: 1 - protein, 120 aa, Artificial Sequence
  Synthesized
QVTLKESGPGLVQPGQTLRLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSS

SEQ ID NO: 2 - DNA, 360 bp, Artificial Sequence
  Synthesized
caggtcacattgaaggaatctggccccggccttgttcagccaggacagacccttaggctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgcgcggatggaactctggtcttactactttgattattgggggcaggggactctcgtcacggtctcgagt

SEQ ID NO: 3 - protein, 120 aa, Artificial Sequence; identical to SEQ ID NO: 1
  Synthesized

SEQ ID NO: 4 - DNA, 360 bp, Artificial Sequence; identical to SEQ ID NO: 2
  Synthesized

SEQ ID NO: 5 - protein, 120 aa, Artificial Sequence; identical to SEQ ID NO: 1
  Synthesized

SEQ ID NO: 6 - DNA, 360 bp, Artificial Sequence; identical to SEQ ID NO: 2
  Synthesized

SEQ ID NO: 7 - protein, 120 aa, Artificial Sequence; identical to SEQ ID NO: 1
  Synthesized

SEQ ID NO: 8 - DNA, 360 bp, Artificial Sequence; identical to SEQ ID NO: 2
  Synthesized

SEQ ID NO: 9 - protein, 120 aa, Artificial Sequence
  Synthesized
EVQLVESGGGLVQPGGSLRLSCVFSGFSLSTSGMGVGWVRQAPGKGLEWVGHIWWDDDKRYNPALKSRFTISRDTSKNTVYLQMNSLRAEDTAVYYCARMELWSYYFDYWGQGTLVTVSS

SEQ ID NO: 10 - DNA, 360 bp, Artificial Sequence
  Synthesized
gaggtgcaacttgtggaaagcggcggcgggttggtgcaacctggcggttcacttcggctctcatgtgtgttcagtggtttttcccttagcacaagcgggatgggtgtcgggtgggtccgccaagcgcctggcaaaggtctggaatgggttggtcacatttggtgggatgatgacaaaaggtataatcccgcgctgaaatctagatttactattagtcgggatacgagtaagaacacggtgtatctgcaaatgaacagtctcagggcagaggatacagcggtatattattgtgctcgaatggagctgtggtcttactattttgattactggggccagggcacgttggtaacggtctcgagt

SEQ ID NO: 11 - protein, 120 aa, Artificial Sequence
  Synthesized
EVQLVESGGGLVQPGGSLRLSCSFSGFSLSTSGMGVGWVRQAPGKGLEWVGHIWWDDDKRYNPALKSRFTISRDTSKNTVYLQMNSLRAEDTAVYYCARMELWSYYFDYWGQGTLVTVSS

SEQ ID NO: 12 - DNA, 360 bp, Artificial Sequence
  Synthesized
gaggtgcaacttgtggaaagcggcggcgggttggtgcaacctggcggttcacttcggctctcatgtagcttcagtggtttttcccttagcacaagcgggatgggtgtcgggtgggtccgccaagcgcctggcaaaggtctggaatgggttggtcacatttggtgggatgatgacaaaaggtataatcccgcgctgaaatctagatttactattagtcgggatacgagtaagaacacggtgtatctgcaaatgaacagtctcagggcagaggatacagcggtatattattgtgctcgaatggagctgtggtcttactattttgattactggggccagggcacgttggtaacggtctcgagt

SEQ ID NO: 13 - protein, 106 aa, Artificial Sequence
  Synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKLEIK

SEQ ID NO: 14 - DNA, 318 bp, Artificial Sequence
  Synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaattggagataaag

SEQ ID NO: 15 - protein, 106 aa, Artificial Sequence
  Synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKITIL

SEQ ID NO: 16 - DNA, 318 bp, Artificial Sequence
  Synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaaattacgatactg

SEQ ID NO: 17 - protein, 106 aa, Artificial Sequence
  Synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKLTVL

SEQ ID NO: 18 - DNA, 318 bp, Artificial Sequence
  Synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaacttacggtactg

SEQ ID NO: 19 - protein, 106 aa, Artificial Sequence
  Synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKVTVL

SEQ ID NO: 20 - DNA, 318 bp, Artificial Sequence
  Synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaagttacggtactg

SEQ ID NO: 21 - protein, 106 aa, Artificial Sequence
  Synthesized
EIVMTQSPSTLSASVGDRVIITCSASSSVSYMHWYQQKPGKAPKLLIYDTSKLASGVPSRFSGSGSGAEFTLTISSLQPDDFATYYCFQGSVYPFTFGQGTKLTVL

SEQ ID NO: 22 - DNA, 318 bp, Artificial Sequence
  Synthesized
gaaatagtgatgacgcagtcacctagcacccttagtgcttctgtaggagacagggttataattacctgcagtgctagttcctcagtgtcatacatgcactggtatcagcagaaaccgggaaaagctccaaagctgcttatatacgacacgtccaaattggcatcaggtgtccccagtcgatttagtggctctggctcaggggctgaatttacgctcacaatctccagcctccaaccagatgacttcgccacatactactgttttcagggctcagtgtatccgtttactttcggccaggggacaaagttgactgtactt

SEQ ID NO: 23 - protein, 106 aa, Artificial Sequence; identical to SEQ ID NO: 21
  Synthesized

SEQ ID NO: 24 - DNA, 318 bp, Artificial Sequence; identical to SEQ ID NO: 22
  Synthesized

SEQ ID NO: 25 - protein, 120 aa, Artificial Sequence
  Synthesized
QVTLKESGPGILQPSQTLSLTCSFSGFSLSTSGMGVGWIRQPSGKGLEWLAHIWWDDDKRYNPALKSRLTISKDTSSNQVFLKIASVDTADTAAYYCARMELWSYYFDYWGQGTTLTVSS

SEQ ID NO: 26 - DNA, 360 bp, Artificial Sequence
  Synthesized
caggtgaccctgaaagaaagcggcccgggcattctgcagccgagccagaccctgagcctgacctgcagctttagcggctttagcctgagcaccagcggcatgggcgtgggctggattcgccagccgagcggcaaaggcctggaatggctggcgcatatttggtgggatgatgataaacgctataacccggcgctgaaaagccgcctgaccattagcaaagataccagcagcaaccaggtgtttctgaaaattgcgagcgtggataccgcggataccgcggcgtattattgcgcgcgcatggaactgtggagctattattttgattattggggccagggcaccaccctgaccgtgagcagc

SEQ ID NO: 27 - protein, 106 aa, Artificial Sequence
  Synthesized
ENVLTQSPAIMSASPGEKVTMTCSASSSVSYMHWYQQKSSTSPKLWIYDTSKLASGVPGRFSGSGSGNSHFLTISSMEAEDVATYYCFQGSVYPFTFGSGTKLEIK

SEQ ID NO: 28 - DNA, 318 bp, Artificial Sequence
  Synthesized
gaaaacgtgctgacccagagcccggcgattatgagcgcgagcccgggcgaaaaagtgaccatgacctgcagcgcgagcagcagcgtgagctatatgcattggtatcagcagaaaagcagcaccagcccgaaactgtggatttatgataccagcaaactggcgagcggcgtgccgggccgctttagcggcagcggcagcggcaacagccattttctgaccattagcagcatggaagcggaagatgtggcgacctattattgctttcagggcagcgtgtatccgtttacctttggcagcggcaccaaactggaaattaaa

SEQ ID NO: 29 - protein, 121 aa, Artificial Sequence
  Synthesized
EVQLVQSGAEVKKPGESLKISCKGSGYSFSSSWIGWVRQAPGKGLEWMGIIYPDDSDTRYSPSFQGQVTISADKSIRTAYLQWSSLKASDTAMYYCARHVTMIWGVIIDFWGQGTLVTVSS

SEQ ID NO: 30 - DNA, 363 bp, Artificial Sequence
  Synthesized
gaggtgcagctggtgcagtctggagcagaggtgaagaaaccaggagagtctctgaagatctcctgtaagggttctggatacagctttagcagttcatggatcggctgggtgcgccaggcacctgggaaaggcctggaatggatggggatcatctatcctgatgactctgataccagatacagtccatccttccaaggccaggtcaccatctcagccgacaagtccatcaggactgcctacctgcagtggagtagcctgaaggcctcggacaccgctatgtattactgtgcgagacatgttactatgatttggggagttattattgacttctggggccagggaaccctggtcaccgtctcctca

SEQ ID NO: 31 - protein, 107 aa, Artificial Sequence
  Synthesized
AIQLTQSPSSLSASVGDRVTITCRASQGISSALAWYQQKPGKAPKLLIYDASSLESGVPSRFSGSGSGTDFTLTISSLQPEDFATYYCQQFNSYPFTFGPGTKVDIK

SEQ ID NO: 32 - DNA, 321 bp, Artificial Sequence
  Synthesized
gccatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccattcactttcggccctgggaccaaagtggatatcaaa

SEQ ID NO: 33 - protein, 449 aa, Artificial Sequence
  Synthesized
QVTLKESGPGILQPSQTLSLTCSFSGFSLSTSGMGVGWIRQPSGKGLEWLAHIWWDDDKRYNPALKSRLTISKDTSSNQVFLKIASVDTADTAAYYCARMELWSYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - DNA, 1347 bp, Artificial Sequence
  Synthesized
caggtgaccctgaaagaaagcggcccgggcattctgcagccgagccagaccctgagcctgacctgcagctttagcggctttagcctgagcaccagcggcatgggcgtgggctggattcgccagccgagcggcaaaggcctggaatggctggcgcatatttggtgggatgatgataaacgctataacccggcgctgaaaagccgcctgaccattagcaaagataccagcagcaaccaggtgtttctgaaaattgcgagcgtggataccgcggataccgcggcgtattattgcgcgcgcatggaactgtggagctattattttgattattggggccagggcaccaccctgaccgtgagcagcgctagcaccaagggcccatctgtcttccccctggcaccctcctccaagagcacctctgggggcacagctgccctgggctgcctggtcaaggactacttccctgaacctgtgacagtgtcctggaactcaggagccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctcctgtagcaggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggcaaa

SEQ ID NO: 35 - protein, 213 aa, Artificial Sequence
  Synthesized
ENVLTQSPAIMSASPGEKVTMTCSASSSVSYMHWYQQKSSTSPKLWIYDTSKLASGVPGRFSGSGSGNSHFLTISSMEAEDVATYYCFQGSVYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - DNA, 639 bp, Artificial Sequence
  Synthesized
gaaaacgtgctgacccagagcccggcgattatgagcgcgagcccgggcgaaaaagtgaccatgacctgcagcgcgagcagcagcgtgagctatatgcattggtatcagcagaaaagcagcaccagcccgaaactgtggatttatgataccagcaaactggcgagcggcgtgccgggccgctttagcggcagcggcagcggcaacagccattttctgaccattagcagcatggaagcggaagatgtggcgacctattattgctttcagggcagcgtgtatccgtttacctttggcagcggcaccaaactggaaattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 37 - protein, 449 aa, Artificial Sequence
  Synthesized
QVTLKESGPGLVQPGQTLRLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - DNA, 1347 bp, Artificial Sequence
  Synthesized
caggtcacattgaaggaatctggccccggccttgttcagccaggacagacccttaggctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgcgcggatggaactctggtcttactactttgattattgggggcaggggactctcgtcacggtgtcctctgctagcaccaagggcccatctgtcttccccctggcaccctcctccaagagcacctctgggggcacagctgccctgggctgcctggtcaaggactacttccctgaacctgtgacagtgtcctggaactcaggagccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctcctgtagcaggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggcaaa

SEQ ID NO: 39 - protein, 213 aa, Artificial Sequence
  Synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - DNA, 639 bp, Artificial Sequence
  Synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaattggagataaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 41 - protein, 254 aa, Artificial Sequence
  Synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKLEIKGGGGSGGGGSGGGGSGGGGSQVTLKESGPGLVQPGQTLRLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGSHHHHHH

SEQ ID NO: 42 - DNA, 765 bp, Artificial Sequence
  Synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaattggagataaagggcggtggcggtagtgggggaggcggttctggcggcggagggtccggcggtggaggatcacaggtcacattgaaggaatctggccccggccttgttcagccaggacagacccttaggctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgcgcggatggaactctggtcttactactttgattattgggggcaggggactctcgtcacggtctcgagtggatcccatcatcaccatcaccattga

SEQ ID NO: 43 - protein, 246 aa, Artificial Sequence
  Synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKLEIKGGGGSGGGGSGGGGSGGGGSQVTLKESGPGLVQPGQTLRLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSS

SEQ ID NO: 44 - DNA, 738 bp, Artificial Sequence
  Synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaattggagataaagggcggtggcggtagtgggggaggcggttctggcggcggagggtccggcggtggaggatcacaggtcacattgaaggaatctggccccggccttgttcagccaggacagacccttaggctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgcgcggatggaactctggtcttactactttgattattgggggcaggggactctcgtcacggtctcgagt

SEQ ID NO: 45 - protein, 246 aa, Artificial Sequence
  Synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKITILGGGGSGGGGSGGGGSGGGGSQVTLKESGPGLVQPGQTLRLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSS

SEQ ID NO: 46 - DNA, 738 bp, Artificial Sequence
  Synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaaattacgatactgggcggtggcggtagtgggggaggcggttctggcggcggagggtccggcggtggaggatcacaggtcacattgaaggaatctggccccggccttgttcagccaggacagacccttaggctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgcgcggatggaactctggtcttactactttgattattgggggcaggggactctcgtcacggtctcgagt

SEQ ID NO: 47 - protein, 246 aa, Artificial Sequence
  Synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKLTVLGGGGSGGGGSGGGGSGGGGSQVTLKESGPGLVQPGQTLRLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSS

SEQ ID NO: 48 - DNA, 738 bp, Artificial Sequence
  Synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaattgacggtactgggcggtggcggtagtgggggaggcggttctggcggcggagggtccggcggtggaggatcacaggtcacattgaaggaatctggccccggccttgttcagccaggacagacccttaggctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgcgcggatggaactctggtcttactactttgattattgggggcaggggactctcgtcacggtctcgagt

SEQ ID NO: 49 - protein, 246 aa, Artificial Sequence
  Synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKVTVLGGGGSGGGGSGGGGSGGGGSQVTLKESGPGLVQPGQTLRLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSS

SEQ ID NO: 50 - DNA, 738 bp, Artificial Sequence
  Synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaagttacggtactgggcggtggcggtagtgggggaggcggttctggcggcggagggtccggcggtggaggatcacaggtcacattgaaggaatctggccccggccttgttcagccaggacagacccttaggctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgcgcggatggaactctggtcttactactttgattattgggggcaggggactctcgtcacggtctcgagt

SEQ ID NO: 51 - protein, 246 aa, Artificial Sequence
  Synthesized
EIVMTQSPSTLSASVGDRVIITCSASSSVSYMHWYQQKPGKAPKLLIYDTSKLASGVPSRFSGSGSGAEFTLTISSLQPDDFATYYCFQGSVYPFTFGQGTKLTVLGGGGSGGGGSGGGGSGGGGSEVQLVESGGGLVQPGGSLRLSCVFSGFSLSTSGMGVGWVRQAPGKGLEWVGHIWWDDDKRYNPALKSRFTISRDTSKNTVYLQMNSLRAEDTAVYYCARMELWSYYFDYWGQGTLVTVSS

SEQ ID NO: 52 - DNA, 738 bp, Artificial Sequence
  Synthesized
gaaatagtgatgacgcagtcacctagcacccttagtgcttctgtaggagacagggttataattacctgcagtgctagttcctcagtgtcatacatgcactggtatcagcagaaaccgggaaaagctccaaagctgcttatatacgacacgtccaaattggcatcaggtgtccccagtcgatttagtggctctggctcaggggctgaatttacgctcacaatctccagcctccaaccagatgacttcgccacatactactgttttcagggctcagtgtatccgtttactttcggccaggggacaaagttgactgtacttggcggtggcggtagtgggggaggcggttctggcggcggagggtccggcggtggaggatcagaggtgcaacttgtggaaagcggcggcgggttggtgcaacctggcggttcacttcggctctcatgtgtgttcagtggtttttcccttagcacaagcgggatgggtgtcgggtgggtccgccaagcgcctggcaaaggtctggaatgggttggtcacatttggtgggatgatgacaaaaggtataatcccgcgctgaaatctagatttactattagtcgggatacgagtaagaacacggtgtatctgcaaatgaacagtctcagggcagaggatacagcggtatattattgtgctcgaatggagctgtggtcttactattttgattactggggccagggcacgttggtaacggtctcgagt

SEQ ID NO: 53 - protein, 246 aa, Artificial Sequence
  Synthesized
EIVMTQSPSTLSASVGDRVIITCSASSSVSYMHWYQQKPGKAPKLLIYDTSKLASGVPSRFSGSGSGAEFTLTISSLQPDDFATYYCFQGSVYPFTFGQGTKLTVLGGGGSGGGGSGGGGSGGGGSEVQLVESGGGLVQPGGSLRLSCSFSGFSLSTSGMGVGWVRQAPGKGLEWVGHIWWDDDKRYNPALKSRFTISRDTSKNTVYLQMNSLRAEDTAVYYCARMELWSYYFDYWGQGTLVTVSS

SEQ ID NO: 54 - DNA, 738 bp, Artificial Sequence
  Synthesized
gaaatagtgatgacgcagtcacctagcacccttagtgcttctgtaggagacagggttataattacctgcagtgctagttcctcagtgtcatacatgcactggtatcagcagaaaccgggaaaagctccaaagctgcttatatacgacacgtccaaattggcatcaggtgtccccagtcgatttagtggctctggctcaggggctgaatttacgctcacaatctccagcctccaaccagatgacttcgccacatactactgttttcagggctcagtgtatccgtttactttcggccaggggacaaagttgactgtacttggcggtggcggtagtgggggaggcggttctggcggcggagggtccggcggtggaggatcagaggtgcaacttgtggaaagcggcggcgggttggtgcaacctggcggttcacttcggctctcatgtagcttcagtggtttttcccttagcacaagcgggatgggtgtcgggtgggtccgccaagcgcctggcaaaggtctggaatgggttggtcacatttggtgggatgatgacaaaaggtataatcccgcgctgaaatctagatttactattagtcgggatacgagtaagaacacggtgtatctgcaaatgaacagtctcagggcagaggatacagcggtatattattgtgctcgaatggagctgtggtcttactattttgattactggggccagggcacgttggtaacggtctcgagt

SEQ ID NO: 55 - protein, 507 aa, Artificial Sequence
  Synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKLEIKGGGGSGGGGSGGGGSGGGGSQVTLKESGPGLVQPGQTLRLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGGSSGSGSGSTGLVPRGSTSSSGTGTSAGTPVAGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVLHEALHNHYTQKSLSLSPGKGSGLNDIFEAQKIEWHE

SEQ ID NO: 56 - DNA, 1521 bp, Artificial Sequence
  Synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaattggagataaagggcggtggcggtagtgggggaggcggttctggcggcggagggtccggcggtggaggatcacaggtcacattgaaggaatctggccccggccttgttcagccaggacagacccttaggctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgcgcggatggaactctggtcttactactttgattattgggggcaggggactctcgtcacggtctcgagtgggggatcctctggaagtggctccggcagcactgggctcgtaccaagggggtccacatccagtagcggtactggcacatccgcgggaacccccgtcgctggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgctagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgagatgccacgtcaaaggcttctatcccagcgacatcgccgtggagtgggagagcaatgggcagccggagaacaactacaagaccacgaagcccgtgctggactccgacggctccttcttcctctacagcaccctcaccgtggacaagagcaggtggcagcaggggaacgtcttctcatgctccgtgctccatgaggctctgcacaaccactacacacagaagagcctctccctgtctccgggcaaaggttcaggcctgaacgatatttttgaagcgcagaaaattgaatggcatgaa

SEQ ID NO: 57 - protein, 507 aa, Artificial Sequence
  Synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKITILGGGGSGGGGSGGGGSGGGGSQVTLKESGPGLVQPGQTLRLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGGSSGSGSGSTGLVPRGSTSSSGTGTSAGTPVAGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVLHEALHNHYTQKSLSLSPGKGSGLNDIFEAQKIEWHE

SEQ ID NO: 58 - DNA, 1521 bp, Artificial Sequence
  Synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaaattacgatactgggcggtggcggtagtgggggaggcggttctggcggcggagggtccggcggtggaggatcacaggtcacattgaaggaatctggccccggccttgttcagccaggacagacccttaggctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgcgcggatggaactctggtcttactactttgattattgggggcaggggactctcgtcacggtctcgagtgggggatcctctggaagtggctccggcagcactgggctcgtaccaagggggtccacatccagtagcggtactggcacatccgcgggaacccccgtcgctggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgctagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgagatgccacgtcaaaggcttctatcccagcgacatcgccgtggagtgggagagcaatgggcagccggagaacaactacaagaccacgaagcccgtgctggactccgacggctccttcttcctctacagcaccctcaccgtggacaagagcaggtggcagcaggggaacgtcttctcatgctccgtgctccatgaggctctgcacaaccactacacacagaagagcctctccctgtctccgggcaaaggttcaggcctgaacgatatttttgaagcgcagaaaattgaatggcatgaa

SEQ ID NO: 59 - protein, 507 aa, Artificial Sequence
  Synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKLTVLGGGGSGGGGSGGGGSGGGGSQVTLKESGPGLVQPGQTLRLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGGSSGSGSGSTGLVPRGSTSSSGTGTSAGTPVAGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVLHEALHNHYTQKSLSLSPGKGSGLNDIFEAQKIEWHE

SEQ ID NO: 60 - DNA, 1521 bp, Artificial Sequence
  Synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaacttacggtactgggcggtggcggtagtgggggaggcggttctggcggcggagggtccggcggtggaggatcacaggtcacattgaaggaatctggccccggccttgttcagccaggacagacccttaggctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgcgcggatggaactctggtcttactactttgattattgggggcaggggactctcgtcacggtctcgagtgggggatcctctggaagtggctccggcagcactgggctcgtaccaagggggtccacatccagtagcggtactggcacatccgcgggaacccccgtcgctggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgctagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgagatgccacgtcaaaggcttctatcccagcgacatcgccgtggagtgggagagcaatgggcagccggagaacaactacaagaccacgaagcccgtgctggactccgacggctccttcttcctctacagcaccctcaccgtggacaagagcaggtggcagcaggggaacgtcttctcatgctccgtgctccatgaggctctgcacaaccactacacacagaagagcctctccctgtctccgggcaaaggttcaggcctgaacgatatttttgaagcgcagaaaattgaatggcatgaa

SEQ ID NO: 61 - protein, 507 aa, Artificial Sequence
  Synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKVTVLGGGGSGGGGSGGGGSGGGGSQVTLKESGPGLVQPGQTLRLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGGSSGSGSGSTGLVPRGSTSSSGTGTSAGTPVAGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVLHEALHNHYTQKSLSLSPGKGSGLNDIFEAQKIEWHE

SEQ ID NO: 62 - DNA, 1521 bp, Artificial Sequence
  Synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaagttacggtactgggcggtggcggtagtgggggaggcggttctggcggcggagggtccggcggtggaggatcacaggtcacattgaaggaatctggccccggccttgttcagccaggacagacccttaggctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgcgcggatggaactctggtcttactactttgattattgggggcaggggactctcgtcacggtctcgagtgggggatcctctggaagtggctccggcagcactgggctcgtaccaagggggtccacatccagtagcggtactggcacatccgcgggaacccccgtcgctggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgctagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgagatgccacgtcaaaggcttctatcccagcgacatcgccgtggagtgggagagcaatgggcagccggagaacaactacaagaccacgaagcccgtgctggactccgacggctccttcttcctctacagcaccctcaccgtggacaagagcaggtggcagcaggggaacgtcttctcatgctccgtgctccatgaggctctgcacaaccactacacacagaagagcctctccctgtctccgggcaaaggttcaggcctgaacgatatttttgaagcgcagaaaattgaatggcatgaa

SEQ ID NO: 63 - protein, 506 aa, Artificial Sequence
  Synthesized
EIVMTQSPSTLSASVGDRVIITCSASSSVSYMHWYQQKPGKAPKLLIYDTSKLASGVPSRFSGSGSGAEFTLTISSLQPDDFATYYCFQGSVYPFTFGQGTKLTVLGGGGSGGGGSGGGGSGGGGSEVQLVESGGGLVQPGGSLRLSCVFSGFSLSTSGMGVGWVRQAPGKGLEWVGHIWWDDDKRYNPALKSRFTISRDTSKNTVYLQMNSLRAEDTAVYYCARMELWSYYFDYWGQGTLVTVSSGSSGSGSGSTGLVPRGSTSSSGTGTSAGTPVAGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVLHEALHNHYTQKSLSLSPGKGSGLNDIFEAQKIEWHE

SEQ ID NO: 64 - DNA, 1518 bp, Artificial Sequence
  Synthesized
gaaatagtgatgacgcagtcacctagcacccttagtgcttctgtaggagacagggttataattacctgcagtgctagttcctcagtgtcatacatgcactggtatcagcagaaaccgggaaaagctccaaagctgcttatatacgacacgtccaaattggcatcaggtgtccccagtcgatttagtggctctggctcaggggctgaatttacgctcacaatctccagcctccaaccagatgacttcgccacatactactgttttcagggctcagtgtatccgtttactttcggccaggggacaaagttgactgtacttggcggtggcggtagtgggggaggcggttctggcggcggagggtccggcggtggaggatcagaggtgcaacttgtggaaagcggcggcgggttggtgcaacctggcggttcacttcggctctcatgtgtgttcagtggtttttcccttagcacaagcgggatgggtgtcgggtgggtccgccaagcgcctggcaaaggtctggaatgggttggtcacatttggtgggatgatgacaaaaggtataatcccgcgctgaaatctagatttactattagtcgggatacgagtaagaacacggtgtatctgcaaatgaacagtctcagggcagaggatacagcggtatattattgtgctcgaatggagctgtggtcttactattttgattactggggccagggcacgttggtaacggtctcgagtggatcctctggaagtggctccggcagcactgggctcgtaccaagggggtccacatccagtagcggtactggcacatccgcgggaacccccgtcgctggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgctagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgagatgccacgtcaaaggcttctatcccagcgacatcgccgtggagtgggagagcaatgggcagccggagaacaactacaagaccacgaagcccgtgctggactccgacggctccttcttcctctacagcaccctcaccgtggacaagagcaggtggcagcaggggaacgtcttctcatgctccgtgctccatgaggctctgcacaaccactacacacagaagagcctctccctgtctccgggcaaaggttcaggcctgaacgatatttttgaagcgcagaaaattgaatggcatgaa

SEQ ID NO: 65 - protein, 506 aa, Artificial Sequence
  Synthesized
EIVMTQSPSTLSASVGDRVIITCSASSSVSYMHWYQQKPGKAPKLLIYDTSKLASGVPSRFSGSGSGAEFTLTISSLQPDDFATYYCFQGSVYPFTFGQGTKLTVLGGGGSGGGGSGGGGSGGGGSEVQLVESGGGLVQPGGSLRLSCSFSGFSLSTSGMGVGWVRQAPGKGLEWVGHIWWDDDKRYNPALKSRFTISRDTSKNTVYLQMNSLRAEDTAVYYCARMELWSYYFDYWGQGTLVTVSSGSSGSGSGSTGLVPRGSTSSSGTGTSAGTPVAGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVLHEALHNHYTQKSLSLSPGKGSGLNDIFEAQKIEWHE

SEQ ID NO: 66 - DNA, 1518 bp, Artificial Sequence
  Synthesized
gaaatagtgatgacgcagtcacctagcacccttagtgcttctgtaggagacagggttataattacctgcagtgctagttcctcagtgtcatacatgcactggtatcagcagaaaccgggaaaagctccaaagctgcttatatacgacacgtccaaattggcatcaggtgtccccagtcgatttagtggctctggctcaggggctgaatttacgctcacaatctccagcctccaaccagatgacttcgccacatactactgttttcagggctcagtgtatccgtttactttcggccaggggacaaagttgactgtacttggcggtggcggtagtgggggaggcggttctggcggcggagggtccggcggtggaggatcagaggtgcaacttgtggaaagcggcggcgggttggtgcaacctggcggttcacttcggctctcatgtagcttcagtggtttttcccttagcacaagcgggatgggtgtcgggtgggtccgccaagcgcctggcaaaggtctggaatgggttggtcacatttggtgggatgatgacaaaaggtataatcccgcgctgaaatctagatttactattagtcgggatacgagtaagaacacggtgtatctgcaaatgaacagtctcagggcagaggatacagcggtatattattgtgctcgaatggagctgtggtcttactattttgattactggggccagggcacgttggtaacggtctcgagtggatcctctggaagtggctccggcagcactgggctcgtaccaagggggtccacatccagtagcggtactggcacatccgcgggaacccccgtcgctggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgctagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgagatgccacgtcaaaggcttctatcccagcgacatcgccgtggagtgggagagcaatgggcagccggagaacaactacaagaccacgaagcccgtgctggactccgacggctccttcttcctctacagcaccctcaccgtggacaagagcaggtggcagcaggggaacgtcttctcatgctccgtgctccatgaggctctgcacaaccactacacacagaagagcctctccctgtctccgggcaaaggttcaggcctgaacgatatttttgaagcgcagaaaattgaatggcatgaa

SEQ ID NO: 67 - protein, 1223 aa, Artificial Sequence
  Synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKVTVLGGGGSGGGGSGGGGSGGGGSQVTLKESGPGLVQPGQTLSLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGGGGSGGGGSQVQLQQSGPGLVKPSETLSITCTVSGFSLTNYGVHWIRQAPGKCLEWLGVIWSGGNTDYNTPFTSRFTITKDNSKNQVYFKLRSVRADDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGRSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 68 - DNA, 3672 bp, Artificial Sequence
  Synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaagtgaccgtcctaggcggtggcggtagtgggggaggcggttctggcggcggagggtccggcggtggaggatcacaggtcacattgaaggaatctggccccggccttgttcagccaggacagacccttagcctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgctcggatggaactctggtcttactactttgattattgggggcaggggactctcgtcacggtctcgagtggcggtggagggtccggcggtggtggatcacaagtacagttgcagcaatccggtcccggtctcgtcaaaccgagtgagacgcttagtataacgtgtactgtttcaggctttagccttacgaactatggagttcactggattcggcaggcacccggcaaatgtttggaatggctgggtgttatttggtcaggtggaaatacagactataacaccccctttacaagtcggttcacaattacgaaagataattccaaaaatcaagtttatttcaagttgagatccgtccgcgcggacgacactgcgatctactattgtgcgagggcactgacctactacgattacgaatttgcgtattgggggcaagggactcttgtaacagtctccagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtgtcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggctccggacggtcgctggtggagtctgggggaggcttggtccagcctggggggtccctgagactctcctgtactgcctctggattcaccatcagtagctaccacatgcagtgggtccgccaggctccagggaaggggctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttcaggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacctggagcctggcgatgctgcaacttactattgtcagtctacctatcttggtactgattatgttggcggtgctttcggcggagggaccaaggtggagatcaaatga

SEQ ID NO: 69 - protein, 740 aa, Artificial Sequence
  Synthesized
EIVMTQSPSTLSASVGDRVIITCQASESISSWLAWYQQKPGKAPKLLIYEASKLASGVPSRFSGSGSGAEFTLTISSLQPDDFATYYCQGYFYFISRTYVNSFGQGTKLTVLGGGGSGGGGSGGGGSGGGGSEVQLVESGGGLVQPGGSLSLSCAASGFTISTNAMSWVRQAPGKGLEWVGVITGRDITYYASWAKGRFTISRDTSKNTVYLQMNSLRAEDTAVYYCARDGGSSAITSNNIWGQGTLVTVSSGGGGSGGGGSEIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGCGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVLGGGGSGGGGSGGGGSGGGGSQVQLQESGGGLVKPGGSLSLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSS

SEQ ID NO: 70 - DNA, 2220 bp, Artificial Sequence
  Synthesized
gaaatcgttatgacgcagagtccctccacgctctccgctagtgtcggggatcgcgtcattatcacatgccaggcctccgagtcaatcagcagctggcttgcatggtatcaacagaagccgggaaaagctcctaaattgctgatctatgaagcgtcaaaattggcgtctggtgtcccatctaggttctccggctctgggtctggtgcggaatttactttgacaatctccagtcttcaaccagacgatttcgctacctactactgccaagggtatttctattttataagccggacatatgtaaactccttcggccaaggaacaaagttgactgttcttggtggcggaggcagtggtggcgggggcagcggaggtggtggttcagggggtggtgggagcgaagtccaattggtagaaagtggcggtggtctggtgcaacctggtggatctcttagcctctcatgcgccgctagtggctttactatttcaactaatgcgatgagctgggttcgccaggcccccggcaaaggacttgagtgggtcggcgtcatcaccggcagggacattacatactatgcgagttgggcaaagggcaggttcacgattagccgcgatacttcaaagaataccgtttaccttcaaatgaatagcttgagggcggaagacacagctgtgtattactgcgcgagggatggaggtagttccgccataacttccaacaacatatggggacaaggcacgctggttactgtctcgagtggcggtggagggtccggcggtggtggatcagaaatcgtccttacacaatctcctagcacactgagtgtgagccccggcgaacgcgcgactttctcttgcagggcaagtcaatccatagggactaatatacattggtatcaacaaaagccaggtaaaccacccaggcttttgattaagtatgcaagtgagtctatttccggtatccctgaccgcttctctggatcaggcagtggcacagagttcacactcaccatatctagtgtgcaatcagaggacttcgccgtgtattactgccaacagaataataactggccgactaccttcggatgcggtacaaagctgaccgttttacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggtggcggtagcggtggcggcggaagtggtggcggaggatcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtcctaggtggaggcggttcaggcggaggtggttccggcggtggcggctccggtggaggcggctctcaggtgcaattgcaggagtcggggggaggcctggtcaagcctggagggtccctgagtctctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctctagc

SEQ ID NO: 71 - protein, 1224 aa, Artificial Sequence
  Synthesized
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVLGGGGSGGGGSGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSGGGGSGGGGSQVTLKESGPGLVQPGQTLRLTCAFSGFSLSTSGMGVGWIRQPPGKCLEWLAHIWWDDDKRYNPALKSRLTISKDTSKNQVYLQMNSLDAEDTAVYYCARMELWSYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGRSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 72 - DNA, 3672 bp, Artificial Sequence
  Synthesized
gaaatcgtccttacacaatctcctagcacactgagtgtgagccccggcgaacgcgcgactttctcttgcagggcaagtcaatccatagggactaatatacattggtatcaacaaaagccaggtaaaccacccaggcttttgattaagtatgcaagtgagtctatttccggtatccctgaccgcttctctggatcaggcagtggcacagagttcacactcaccatatctagtgtgcaatcagaggacttcgccgtgtattactgccaacagaataataactggccgactaccttcggacccggtacaaagctgaccgttttaggcggtggcggtagtgggggaggcggttctggcggcggagggtccggcggtggaggatcacaagtacagttgcagcaatccggtcccggtctcgtcaaaccgagtgagacgcttagtataacgtgtactgtttcaggctttagccttacgaactatggagttcactggattcggcaggcacccggcaaaggtttggaatggctgggtgttatttggtcaggtggaaatacagactataacaccccctttacaagtcggttcacaattacgaaagataattccaaaaatcaagtttatttcaagttgagatccgtccgcgcggacgacactgcgatctactattgtgcgagggcactgacctactacgattacgaatttgcgtattgggggcaagggactcttgtaacagtctcgagcggcggtggagggtccggcggtggtggatcacaggtcacattgaaggaatctggccccggccttgttcagccaggacagacccttaggctcacctgtgccttcagtggtttttctcttagcactagcggtatgggggtcggctggattcggcagcctcccggcaaatgtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgctcggatggaactctggtcttactactttgattattgggggcaggggactctcgtcacggtgtcgagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtgtcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggctccggacggtcgctggtggagtctgggggaggcttggtccagcctggggggtccctgagactctcctgtactgcctctggattcaccatcagtagctaccacatgcagtgggtccgccaggctccagggaaggggctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttcaggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacctggagcctggcgatgctgcaacttactattgtcagtctacctatcttggtactgattatgttggcggtgctttcggcggagggaccaaggtggagatcaaa

SEQ ID NO: 73 - protein, 739 aa, Artificial Sequence
  Synthesized
EIVMTQSPSTLSASVGDRVIITCQASESISSWLAWYQQKPGKAPKLLIYEASKLASGVPSRFSGSGSGAEFTLTISSLQPDDFATYYCQGYFYFISRTYVNSFGQGTKLTVLGGGGSGGGGSGGGGSGGGGSEVQLVESGGGLVQPGGSLSLSCAASGFTISTNAMSWVRQAPGKGLEWVGVITGRDITYYASWAKGRFTISRDTSKNTVYLQMNSLRAEDTAVYYCARDGGSSAITSNNIWGQGTLVTVSSGGGGSGGGGSENVLTQSPASLSASPGERVTITCSASSSVSYMHWYQQKPGQAPKLWIYDTSKLASGVPSRFSGSGSGNDHTLTISSMEPEDFATYYCFQGSVYPFTFGCGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVLGGGGSGGGGSGGGGSGGGGSQVQLQESGGGLVKPGGSLSLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSS

SEQ ID NO: 74 - DNA, 2217 bp, Artificial Sequence
  Synthesized
gaaatcgttatgacgcagagtccctccacgctctccgctagtgtcggggatcgcgtcattatcacatgccaggcctccgagtcaatcagcagctggcttgcatggtatcaacagaagccgggaaaagctcctaaattgctgatctatgaagcgtcaaaattggcgtctggtgtcccatctaggttctccggctctgggtctggtgcggaatttactttgacaatctccagtcttcaaccagacgatttcgctacctactactgccaagggtatttctattttataagccggacatatgtaaactccttcggccaaggaacaaagttgactgttcttggtggcggaggcagtggtggcgggggcagcggaggtggtggttcagggggtggtgggagcgaagtccaattggtagaaagtggcggtggtctggtgcaacctggtggatctcttagcctctcatgcgccgctagtggctttactatttcaactaatgcgatgagctgggttcgccaggcccccggcaaaggacttgagtgggtcggcgtcatcaccggcagggacattacatactatgcgagttgggcaaagggcaggttcacgattagccgcgatacttcaaagaataccgtttaccttcaaatgaatagcttgagggcggaagacacagctgtgtattactgcgcgagggatggaggtagttccgccataacttccaacaacatatggggacaaggcacgctggttactgtctcgagcggcggtggagggtccggcggtggtggatcagaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggtgtgggacaaaagtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggtggcggtagcggtggcggcggaagtggtggcggaggatcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtcctaggtggaggcggttcaggcggaggtggttccggcggtggcggctccggtggaggcggctctcaggtgcaattgcaggagtcggggggaggcctggtcaagcctggagggtccctgagtctctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctctagc

SEQ ID NO: 75 - protein, 1234 aa, Artificial Sequence
  Synthesized
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVLGGGGSGGGGSGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSGGGGSGGGGSGGGGSGGGGSEVQLVESGGGLVQPGGSLRLSCAASGFTISTNAMSWVRQAPGKCLEWIGVITGRDITYYASWAKGRFTISRDNSKNTLYLQMNSLRAEDTAVYYCARDGGSSAITSNNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGRSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 76 - DNA, 3702 bp, Artificial Sequence
  Synthesized
gaaatcgtccttacacaatctcctagcacactgagtgtgagccccggcgaacgcgcgactttctcttgcagggcaagtcaatccatagggactaatatacattggtatcaacaaaagccaggtaaaccacccaggcttttgattaagtatgcaagtgagtctatttccggtatccctgaccgcttctctggatcaggcagtggcacagagttcacactcaccatatctagtgtgcaatcagaggacttcgccgtgtattactgccaacagaataataactggccgactaccttcggacccggtacaaagctgaccgttttaggcggtggcggtagtgggggaggcggttctggcggcggagggtccggcggtggaggatcacaagtacagttgcagcaatccggtcccggtctcgtcaaaccgagtgagacgcttagtataacgtgtactgtttcaggctttagccttacgaactatggagttcactggattcggcaggcacccggcaaaggtttggaatggctgggtgttatttggtcaggtggaaatacagactataacaccccctttacaagtcggttcacaattacgaaagataattccaaaaatcaagtttatttcaagttgagatccgtccgcgcggacgacactgcgatctactattgtgcgagggcactgacctactacgattacgaatttgcgtattgggggcaagggactcttgtaacagtctcgagcggtggaggcggatctggcggaggtggttccggcggtggcggctccggtggaggcggctctgaggtgcagctggtggagtctgggggaggcttggtccagcctggggggtccctgagactctcctgtgcagcctctggattcaccatcagtaccaatgcaatgagctgggtccgccaggctccagggaagtgtctggagtggatcggagtcattactggtcgtgatatcacatactacgcgagctgggcgaaaggcagattcaccatctccagagacaattccaagaacacgctgtatcttcaaatgaacagcctgagagccgaggacacggctgtgtattactgtgcgagagacggtggttcttctgctattactagtaacaacatttggggccagggaaccctggtcaccgtgtcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtgtcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggctccggacggtcgctggtggagtctgggggaggcttggtccagcctggggggtccctgagactctcctgtactgcctctggattcaccatcagtagctaccacatgcagtgggtccgccaggctccagggaaggggctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttcaggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacctggagcctggcgatgctgcaacttactattgtcagtctacctatcttggtactgattatgttggcggtgctttcggcggagggaccaaggtggagatcaaa

SEQ ID NO: 77 - protein, 739 aa, Artificial Sequence
  Synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKVTVLGGGGSGGGGSGGGGSGGGGSQVTLKESGPGLVQPGQTLSLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGGGGSGGGGSDVVMTQSPSTLSASVGDRVTINCQASESISSWLAWYQQKPGKAPKLLIYEASKLASGVPSRFSGSGSGTEFTLTISSLQPDDFATYYCQGYFYFISRTYVNSFGCGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVLGGGGSGGGGSGGGGSGGGGSQVQLQESGGGLVKPGGSLSLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSS

SEQ ID NO: 78 - DNA, 2220 bp, Artificial Sequence
  Synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaagtgaccgtcctaggcggtggcggtagtgggggaggcggttctggcggcggagggtccggcggtggaggatcacaggtcacattgaaggaatctggccccggccttgttcagccaggacagacccttagcctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgctcggatggaactctggtcttactactttgattattgggggcaggggactctcgtcacggtctcgagtggcggtggagggtccggcggtggtggatcagacgtcgtgatgacccagtctccttccaccctgtctgcatctgtaggagacagagtcaccatcaattgccaagccagtgagagcattagcagttggttagcctggtatcagcagaaaccagggaaagcccctaagctcctgatctatgaagcatccaaactggcatctggggtcccatcaaggttcagcggcagtggatctgggacagaatttactctcaccatcagcagcctgcagcctgatgattttgcaacttattactgccaaggctatttttattttattagtcgtacttatgtaaattctttcggctgtgggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggtggcggtagcggtggcggcggaagtggtggcggaggatcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtcctaggtggaggcggttcaggcggaggtggttccggcggtggcggctccggtggaggcggctctcaggtgcaattgcaggagtcggggggaggcctggtcaagcctggagggtccctgagtctctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctctagctga

SEQ ID NO: 79 - protein, 1230 aa, Artificial Sequence
  Synthesized
DVVMTQSPSTLSASVGDRVTINCQASESISSWLAWYQQKPGKAPKLLIYEASKLASGVPSRFSGSGSGTEFTLTISSLQPDDFATYYCQGYFYFISRTYVNSFGGGTKVEIKGGGGSGGGGSGGGGSGGGGSEVQLVESGGGLVQPGGSLRLSCAASGFTISTNAMSWVRQAPGKGLEWIGVITGRDITYYASWAKGRFTISRDNSKNTLYLQMNSLRAEDTAVYYCARDGGSSAITSNNIWGQGTLVTVSSGGGGSGGGGSEVQLVQSGAEVKKPGESLKISCKGSGYSFSSSWIGWVRQAPGKGLEWMGIIYPDDSDTRYSPSFQGQVTISADKSIRTAYLQWSSLKASDTAMYYCARHVTMIWGVIIDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSRSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 80 - DNA, 3690 bp, Artificial Sequence
  Synthesized
gacgtcgtgatgacccagtctccttccaccctgtctgcatctgtaggagacagagtcaccatcaattgccaagccagtgagagcattagcagttggttagcctggtatcagcagaaaccagggaaagcccctaagctcctgatctatgaagcatccaaactggcatctggggtcccatcaaggttcagcggcagtggatctgggacagagttcactctcaccatcagcagcctgcagcctgatgattttgcaacttattactgccaaggctatttttattttattagtcgtacttatgtaaattctttcggcggagggaccaaggtggagatcaaaggcggtggcggtagtgggggaggcggttctggcggcggagggtccggcggtggaggatcagaggtgcagctggtggagtctgggggaggcttggtccagcctggggggtccctgagactctcctgtgcagcctctggattcaccatcagtaccaatgcaatgagctgggtccgccaggctccagggaaggggctggagtggatcggagtcattactggtcgtgatatcacatactacgcgagctgggcgaaaggcagattcaccatctccagagacaattccaagaacacgctgtatcttcaaatgaacagcctgagagccgaggacacggctgtgtattactgtgcgcgcgacggtggatcatctgctattactagtaacaacatttggggccaaggaactctggtcaccgtttcttcaggcggtggagggtccggcggtggtggatccgaggtgcagctggtgcagtctggagcagaggtgaagaaaccaggagagtctctgaagatctcctgtaagggttctggatacagctttagcagttcatggatcggctgggtgcgccaggcacctgggaaaggcctggaatggatggggatcatctatcctgatgactctgataccagatacagtccatccttccaaggccaggtcaccatctcagccgacaagtccatcaggactgcctacctgcagtggagtagcctgaaggcctcggacaccgctatgtattactgtgcgagacatgttactatgatttggggagttattattgacttctggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtataccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtctcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggatcccggtcgctggtggagtctgggggaggcttggtccagcctggggggtccctgagactctcctgtacagcctctggattcaccatcagtagctaccacatgcagtgggtccgccaggctccagggaaggggctggagtacatcggaaccattagtagtggtggtaatgtatactacgcgagctccgcgagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcgagcggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacctggagcctggcgatgctgcaacttactattgtcagtctacctatcttggtactgattatgttggcggtgctttcggcggagggaccaaggtggagatcaaa

SEQ ID NO: 81 - protein, 214 aa, Artificial Sequence
  Synthesized
AIQLTQSPSSLSASVGDRVTITCRASQGISSALAWYQQKPGKAPKLLIYDASSLESGVPSRFSGSGSGTDFTLTISSLQPEDFATYYCQQFNSYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - DNA, 642 bp, Artificial Sequence
  Synthesized
gccatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccattcactttcggccctgggaccaaagtggatatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 83 - protein, 450 aa, Artificial Sequence
  Synthesized
EVQLVQSGAEVKKPGESLKISCKGSGYSFSSSWIGWVRQAPGKGLEWMGIIYPDDSDTRYSPSFQGQVTISADKSIRTAYLQWSSLKASDTAMYYCARHVTMIWGVIIDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 84 - DNA, 1350 bp, Artificial Sequence
  Synthesized
gaggtgcagctggtgcagtctggagcagaggtgaagaaaccaggagagtctctgaagatctcctgtaagggttctggatacagctttagcagttcatggatcggctgggtgcgccaggcacctgggaaaggcctggaatggatggggatcatctatcctgatgactctgataccagatacagtccatccttccaaggccaggtcaccatctcagccgacaagtccatcaggactgcctacctgcagtggagtagcctgaaggcctcggacaccgctatgtattactgtgcgagacatgttactatgatttggggagttattattgacttctggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 85 - protein, 214 aa, Artificial Sequence; identical to SEQ ID NO: 81
  Synthesized

SEQ ID NO: 86 - DNA, 642 bp, Artificial Sequence; identical to SEQ ID NO: 82
  Synthesized

SEQ ID NO: 87 - protein, 1225 aa, Artificial Sequence
  Synthesized
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLTVLGGGGSGGGGSGGGGSGGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSSGGGGSGGGGSEVQLVESGGGLVQPGGSLRLSCAASGFTISTNAMSWVRQAPGKGLEWIGVITGRDITYYASWAKGRFTISRDNSKNTLYLQMNSLRAEDTAVYYCARDGGSSAITSNNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGRSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 88 - DNA, 3678 bp, Artificial Sequence
  Synthesized
cagatcgtgctgagccagagccccgccatcctgagcgccagccccggcgagaaggtgaccatgacctgccgggccagcagcagcgtgagctacatccactggttccagcagaagcccggcagcagccccaagccctggatctacgccaccagcaacctggccagcggcgtgcccgtgcggttcagcggcagcggcagcggcaccagctacagcctgaccatcagccgggtggaggccgaggacgccgccacctactactgccagcagtggaccagcaacccccccaccttcggcggcggcaccaagctgaccgtgctgggtggtggtggctctggaggaggcgggagcgggggtggtggctcaggtggtggaggttcccaggtgcagctgcagcagcccggcgccgagctggtgaagcccggcgccagcgtgaagatgagctgcaaggccagcggctacaccttcaccagctacaacatgcactgggtgaagcagacccccggccggggcctggagtggatcggcgccatctaccccggcaacggcgacaccagctacaaccagaagttcaagggcaaggccaccctgaccgccgacaagagcagcagcaccgcctacatgcagctgagcagcctgaccagcgaggacagcgccgtgtactactgcgcccggagcacctactacggcggcgactggtacttcaacgtgtggggcgccggcaccaccgtgaccgtctcgagtggcggtggagggtccggcggtggtggatcagaggtgcagctggtggagtctgggggaggcttggtccagcctggggggtccctgagactctcctgtgcagcctctggattcaccatcagtaccaatgcaatgagctgggtccgccaggctccagggaaggggctggagtggatcggagtcattactggtcgtgatatcacatactacgcgagctgggcgaaaggcagattcaccatctccagagacaattccaagaacacgctgtatcttcaaatgaacagcctgagagccgaggacacggctgtgtattactgtgcgagagacggtggttcttctgctattactagtaacaacatttggggccagggaaccctggtcaccgtgtcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtgtcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggctccggacggtcgctggtggagtctgggggaggcttggtccagcctggggggtccctgagactctcctgtactgcctctggattcaccatcagtagctaccacatgcagtgggtccgccaggctccagggaaggggctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttcaggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacctggagcctggcgatgctgcaacttactattgtcagtctacctatcttggtactgattatgttggcggtgctttcggcggagggaccaaggtggagatcaaatga

SEQ ID NO: 89 - protein, 475 aa, Artificial Sequence
  Synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKVTVLGGGGSGGGGSGGGGSGGGGSQVTLKESGPGLVQPGQTLRLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGGGGSGGGGSDVVMTQSPSTLSASVGDRVTINCQASESISSWLAWYQQKPGKAPKLLIYEASKLASGVPSRFSGSGSGTEFTLTISSLQPDDFATYYCQGYFYFISRTYVNSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - DNA, 1425 bp, Artificial Sequence
  Synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaagtgacggtactgggtggaggcggttcaggcggaggtggttccggcggtggcggctccggtggaggcggctctcaggtcacattgaaggaatctggccccggccttgttcagccaggacagacccttaggctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgcgcggatggaactctggtcttactactttgattattgggggcaggggactctcgtcacggtctcgagcggcggtggagggtccggcggtggtggatcagacgtcgtgatgacccagtctccttccaccctgtctgcatctgtaggagacagagtcaccatcaattgccaagccagtgagagcattagcagttggttagcctggtatcagcagaaaccagggaaagcccctaagctcctgatctatgaagcatccaaactggcatctggggtcccatcaaggttcagcggcagtggatctgggacagaattcactctcaccatcagcagcctgcagcctgatgattttgcaacttattactgccaaggctatttttattttattagtcgtacttatgtaaattctttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 91 - protein, 120 aa, Artificial Sequence
  Synthesized
QVTLKESGPGLVQPGQTLRLTCAFSGFSLSTSGMGVGWIRQPPGKCLEWLAHIWWDDDKRYNPALKSRLTISKDTSKNQVYLQMNSLDAEDTAVYYCARMELWSYYFDYWGQGTLVTVSS

SEQ ID NO: 92 - DNA, 360 bp, Artificial Sequence
  Synthesized
caggtcacattgaaggaatctggccccggccttgttcagccaggacagacccttaggctcacctgtgccttcagtggtttttctcttagcactagcggtatgggggtcggctggattcggcagcctcccggcaaatgtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgctcggatggaactctggtcttactactttgattattgggggcaggggactctcgtcacggtgtcgagt

SEQ ID NO: 93 - protein, 106 aa, Artificial Sequence
  Synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGCGTKVEIK

SEQ ID NO: 94 - DNA, 318 bp, Artificial Sequence
  Synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggtgtgggacaaaagtggagatcaag

SEQ ID NO: 95 - protein, 246 aa, Artificial Sequence
  Synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGCGTKVEIKGGGGSGGGGSGGGGSGGGGSQVTLKESGPGLVQPGQTLRLTCAFSGFSLSTSGMGVGWIRQPPGKCLEWLAHIWWDDDKRYNPALKSRLTISKDTSKNQVYLQMNSLDAEDTAVYYCARMELWSYYFDYWGQGTLVTVSS

SEQ ID NO: 96 - DNA, 738 bp, Artificial Sequence
  Synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggtgtgggacaaaagtggagatcaagggtggcggaggcagtggtggcgggggcagcggaggtggtggttcagggggtggtgggagccaggtcacattgaaggaatctggccccggccttgttcagccaggacagacccttaggctcacctgtgccttcagtggtttttctcttagcactagcggtatgggggtcggctggattcggcagcctcccggcaaatgtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgctcggatggaactctggtcttactactttgattattgggggcaggggactctcgtcacggtgtcgagt